(19) United States
(12) Patent Application Publication
(10) Pub. No.: US 2004/0038982 A1
(43) Pub. Date: Feb. 26, 2004

(54) Compounds and methods

(76) Inventors: William E. Bondinell, Collegeville, PA (US); Michael J. Neeb, Collegeville, PA (US)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/343,880

(22) PCT Filed: Jul. 13, 2001
(86) PCT No.: PCT/US01/22529

(51) Int. Cl. A61K 31/55; A61K 31/541; A61K 31/5377; A61K 31/496; A61K 31/454

(52) U.S. Cl. 514/252.13; 514/217.05; 514/227.8; 514/235.8; 514/253.01; 514/254.01; 514/254.02; 514/254.05; 514/326

(57) ABSTRACT

This invention relates to substituted heterocyclic compounds which are modulators, agonists or antagonists, of the CCR5 receptor. In addition, this invention relates to the treatment and prevention of disease states mediated by CCR5, including, but not limited to, asthma and atopic disorders (for example, atopic dermatitis and allergies), rheumatoid arthritis, sarcoidosis, or idiopathic pulmonary fibrosis and other fibrotic diseases, atherosclerosis, psoriasis, autoimmune diseases such as multiple sclerosis, treating and/or preventing rejection of transplanted organs, and inflammatory bowel disease, all in mammals, by the use of substituted heterocyclic compounds which are CCR5 receptor antagonists. Furthermore, since CD8+ T cells have been implicated in COPD, CCR5 may play a role in their recruitment and therefore antagonists to CCR5 could provide potential therapeutic in the treatment of COPD. Also, since CCR5 is a co-receptor for the entry of HIV into cells, selective receptor modulators may be useful in the treatment of HIV infection.

### FIELD OF THE INVENTION

This invention relates to substituted heterocyclic compounds which are modulators, agonists or antagonists, of the CC chemokine receptor CC-CKR5 now designated as CCR5 (Nature Medicine 1996, 2, 1174-8). In addition, this invention relates to the treatment and prevention of disease states mediated by CCR5.

### BACKGROUND OF THE INVENTION

T cells are not only key regulators of the immune response to infectious agents but are believed critical for the initiation and maintenance of the inflammatory reaction in a variety of chronic diseases. Increased numbers or enhanced activation state of T cells, especially CD4+ T cells, have been demonstrated in the synovium of individuals with rheumatoid arthritis (M. J. Elliott and R. N. Maini, Int. Arch. Allergy Immunol. 104: 112-1125, 1994), in the bronchial mucosa of asthmatics (C. J. Corrigan and A. B. Kay, Immunol. Today 13:501-506, 1992), in the lesions of multiple sclerosis (R. Martin and H. F. McFarland, Crit. Rev. Clin. Lab. Sci. 32: 121-182, 1995), in psoriatic lesions (J. L. Jones, J. Berth-Jone, A. Fletcher and P. E. Hutchinson, J. Pathol. 174: 77-82, 1994) and in the fatty streaks of atherosclerosis (R. Ross, Annu. Rev. Physiol. 57: 791-804, 1995).

T cells, as well as other inflammatory cells, will migrate into tissues in response to the production of a variety of chemotactic factors. Among these factors are a superfamily of 8-12 kDa proteins known as the chemokines. These proteins share structural features such as the presence of 3-4 conserved cysteine residues. RANTES, which stands for Regulated upon Activation Normal T cell Expressed and Secreted, is an 8 kDa protein member of CC branch of the chemokine family. These proteins recruit and activate immune and inflammatory cells through an interaction with G-protein coupled receptors. The CC branch is defined by the absence of an intervening amino acid residue between the first two cysteine residues and members of this family predominately elicit the migration of mononuclear cells, eosinophils and basophils (M. Baggiolini, B. Dewald, and B. Moser, Adv. Immunol. 55: 97-179, 1994; and J. J. Oppenheim, C. O. C. Zachariae, N. Mukaida, and K. Matsushima, Annu. Rev. Immunol. 9: 617-648, 1991).

RANTES potently produces chemotaxis of T cells, basophils, eosinophils, monocytes and mast cells. RANTES was originally identified as gene product induced late after antigen activation of T-cells (T. J. Schall, J. Jongstra, B. J. Dyer, J. Jorgensen, et al., J. Immunol. 141:1018-1025, 1988), however, RANTES has been shown to be synthesized and secreted by a diverse group of cells that include epithelial and endothelial cells (C. Stellato, L. A. Beck, G. A. Gorgone, D. Proud, et al., J. Immunol. 155: 410-418, 1995; and A. Marfaing-Koka, O. Devergne, G. Gorgone, A. Portier, et al., J. Immunol. 154: 1870-1878, 1994), synovial fibroblasts (P. Rathanaswami, M. Hachicha, M. Sadick, T. J. Schall, et al., J. Biol. Chem. 268: 5834-5839, 1993) and dermal fibroblasts (M. Sticherling, M. Kupper, F. Koltrowitz, E. Bornscheuer, et al., (J. Invest. Dermatol. 105: 585-591, 1995), mesangial cells (G. Wolf, S. Aberle, F. Thaiss, et al., Kidney Int. 44: 795-804, 1994) and platelets (Y. Koameyoshi, A. Dorschner, A. I. Mallet, E. Christophers, et al., J. Exp. Med. 176: 587-592, 1992). In these cells, RANTES mRNA is rapidly upregulated in response to IL-1 or TNF•. Although RANTES mRNA is not usually detected in normal tissues (J. M. Pattison, P. J. Nelson, and A. M. Krensky, Clin. Immunother. 4: 1-8, 1995), increased mRNA or protein has been found in diseases characterized by a mononuclear infiltrate. For example, RANTES mRNA was visualized using in situ hybridization in renal allografts undergoing rejection (J. M. Pattison, P. J. Nelson, and A. M. Krensky, Clin. Immunother. 4: 1-8, 1995; and K. C. Nadeau, H. Azuma and N. I. Tilney, Proc. Natl. Acad. USA 92: 8729-8733, 1995) in the skin of atopic dermatitis patients after exposure to antigen (S. Ying, L. Taborda-Barata, Q. Meng, M. Humbert, et al., J. Exp. Med. 181: 2153-2159, 1995), and in endothelial cells of coronary arteries undergoing accelerated atherosclerosis after cardiac transplant (J. M. Pattison, P. J. Nelson, and A. M. Krensky, Clin. Immunother. 4: 1-8, 1995). Further, increased immunoreactive protein for RANTES has been detected in bronchoalveolar lavage fluid (R. Alam, J. York, M. Boyers, et al., Am. J. Resp. Crit. Care Med. 149: A951, 1994) and sputum from asthmatic individuals (C. M. Gelder, P. S. Thomas, D. H. Yates, I. M. Adcock, et al., Thorax 50: 1033-1037, 1995).

Several receptors have been identified that bind RANTES. In particular, CCR5, when expressed in either HEK 293 cells or CHO cells, binds RANTES. This receptor is expressed in T-cells and in monocytes and macrophages, immune/inflammatory cells which are important in the maintenance of a chronic inflammatory reaction. Pharmacological characterization of CCR5 indicates similarities to the RANTES binding site observed on isolated T cells. Therefore, antagonism of RANTES' action on CCR5, as well as antagonism of other natural modulators of CCR5, should inhibit the recruitment and activation of T cells and macrophages into inflammatory lesions and provide a novel therapeutic approach for the treatment of atopic and autoimmune disorders.

Since T cells express CCR5, selective receptor modulators of CCR5, particularly antagonists, are likely to provide beneficial effects in diseases including, but not limited to, asthma and atopic disorders (for example, atopic dermatitis and allergies), rheumatoid arthritis, sarcoidosis, or idiopathic pulmonary fibrosis and other fibrotic diseases, atherosclerosis, psoriasis, autoimmune diseases such as multiple sclerosis, treating and/or preventing rejection of transplanted organs, and inflammatory bowel disease, all in mammals, preferably humans. Furthermore, since CD8+ T cells have been implicated in chronic obstructive pulmonary disease (COPD), CCR5 may play a role in their recruitment and therefore antagonists to CCR5 could provide potential therapeutic activity in the treatment of COPD. Also, since CCR5 is a co-receptor for the entry of HIV into cells, selective receptor modulators may be useful in the treatment of HIV infection.

Compounds formula (I) having 5-HT_{1D/1B} receptor antagonist activity have been reported in FR 2758328, published Jul. 17, 1998; FR 2761069, published Sep. 25, 1998; Matzen et al., J. Med. Chem. 2000, 43, 1149-1157; DE 197 56 036 A1, published Jun. 24, 1999; WO 96/02525, published Feb. 1, 1996; WO 97/28140, published Aug. 7, 1997; WO 97/28141, published Aug. 7, 1997; WO 98/31677, published Jul. 23, 1998; U.S. Pat. No. 5,789,412, issued Aug. 4, 1998; WO 95/29907, published Nov. 9, 1995; or compounds which inhibit leukotriene synthesis have been reported in WO 97/24328, published Jul. 10, 1997; or compounds which antagonize tocolytic oxytocin receptor antagonist activity have been reported in WO 94/07496, published Apr. 14, 1994, and WO 95/25443, published Sep. 28, 1995.

Surprisingly, it has now been discovered that this class of non-peptide compounds, in particular substituted heterocyclic compounds of formula (I), function as CCR5 receptor modulators, and therefore, have utility in the treatment and prevention of disease states mediated by CCR5 receptor mechanisms.

### SUMMARY OF THE INVENTION

The present invention is to compounds of formula (I), or a pharmaceutically acceptable salt, or solvate thereof, and their use as CCR5 modulators for the treatment and/or prophylaxis of certain disease states, including, but not limited to, COPD, asthma and atopic disorders (for example, atopic dermatitis and allergies), rheumatoid arthritis, sarcoidosis, or idiopathic pulmonary fibrosis and other fibrotic diseases, atherosclerosis, psoriasis, autoimmune diseases such as multiple sclerosis, treating and/or preventing rejection of transplanted organs, inflammatory bowel disease, and HIV infection, all in mammals, preferably humans. The preferred compounds for use as CCR5 modulators are those compounds of Formula (I) as noted herein.

In addition, the present invention is directed to a method of preventing or treating CCR5-mediated diseases in a mammal, preferably a human, by administering to the mammal an effective amount of a CCR5 receptor ligand, or a pharmaceutically acceptable salt or solvate thereof.

Further, the present invention is directed to methods for making and using the compounds of formula (I), as well as pharmaceutical compositions of formula (I) or a pharmaceutically acceptable salts or solvates thereof.

Yet further, the present invention is directed to the use of a CCR5 receptor ligand in the manufacture of a medicament for the prophylaxis or treatment of certain disease states, including, but not limited to, COPD, asthma and atopic disorders (for example, atopic dermatitis and allergies), rheumatoid arthritis, sarcoidosis, or idiopathic pulmonary fibrosis and other fibrotic diseases, atherosclerosis, psoriasis, autoimmune diseases such as multiple sclerosis, treating and/or preventing rejection of transplanted organs, inflammatory bowel disease, and HIV infection, for example in a mammal such as a human.

Still further, the present invention is directed to a CCR5 receptor ligand, or a pharmaceutically acceptable salt, or solvate thereof, for use in the prophylaxis or treatment of certain disease states, including, but not limited to, COPD, asthma and atopic disorders (for example, atopic dermatitis and allergies), rheumatoid arthritis, sarcoidosis, or idiopathic pulmonary fibrosis and other fibrotic diseases, atherosclerosis, psoriasis, autoimmune diseases such as multiple sclerosis, treating and/or preventing rejection of transplanted organs, inflammatory bowel disease, and HIV infection, for example in a mammal such as a human.

The present invention is also directed to combined therapy to prevent and treat inflammatory and immunoregulatory disorders or diseases, including asthma and allergic diseases, as well as rheumatoid arthritis and atherosclerosis, and those pathologies noted above, and is illustrated by the combination of the compounds of this invention and other compounds which are know for such utilities.

The present invention is further directed to combinations of the present compounds of formula (I) with one or more agents useful in the prevention or treatment of AIDS. For example, the compounds of this invention may be effectively administered, whether at periods of pre-exposure and/or post-exposure, in combination with effective amounts of the AIDS antivirals, immunomodulators, anti-infectives, or vaccines known to the skilled artisan.

### DETAILED DESCRIPTION OF THE INVENTION

It has now been discovered that substituted heterocycles of formula (I) are CCR5 receptor modulators. It has also now been discovered that selective inhibition of CCR5 receptor mechanisms by treatment with the receptor modulators of formula (I), or a pharmaceutically acceptable salt thereof, represents a novel therapeutic and preventative approach to the treatment of a variety of disease states, including, but not limited to, asthma and atopic disorders (for example, atopic dermatitis and allergies), rheumatoid arthritis, sarcoidosis, or idiopathic pulmonary fibrosis and other fibrotic diseases, atherosclerosis, psoriasis, autoimmune diseases such as multiple sclerosis, treating and/or preventing rejection of transplanted organs, and inflammatory bowel disease, all in mammals, preferably humans. Furthermore, since CD8+ T cells have been implicated in COPD, CCR5 may play a role in their recruitment and therefore antagonists to CCR5 could provide potential therapeutic in the treatment of COPD. Also, since CCR5 is a co-receptor for entry into cells, selective receptor modulators may be useful in the treatment of HIV infection.

Compounds of formula (I) for use herein as CCR5 modulators include those compounds as described in FR 2758328, published Jul. 17, 1998, FR 2761069, published Sep. 25, 1998, WO 94/07496, published Apr. 14, 1994, WO 95/25443, published Sep. 28, 1995, and PCT/US00/01908, filed Jan. 25, 2000. Each of these references is incorporated herein in their entirety.

Preferred compounds for use as CCR5 modulators are those compounds of formula (I) as noted herein.

A preferred group of compounds for use herein are those compounds of the formula (I) or a pharmaceutically acceptable salt or solvate thereof:

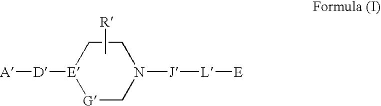

in which:

the basic nitrogen in moiety E may be optionally quaternized with C₁₋₆alkyl or is optionally present as the N-oxide;

A′ is aryl or heteroaryl, each of which is optionally substituted with one or more of R^{1′}; or A′ is aryl or heteroaryl fused to a saturated or partly unsaturated 5-7-membered ring to form a higher order ring moiety, which ring moiety optionally contains 1 or 2 heteroatoms selected from oxygen, nitrogen or sulfur, wherein nitrogen may be optionally substituted with hydrogen, C₁₋₆alkyl or C₃₋₇cycloalkyl; wherein the higher order ring moiety is optionally substituted with one or more of R^{1′},

R^{1′} is hydrogen, C₁₋₆alkyl, C₂₋₆alkenyl, C₂₋₆alkynyl, C₃₋₇cycloalkyl, C₃₋₆cycloalkenyl, CH₂CF₃, aryl, aralkyl, (CH₂)_{a′}NR^{2′}R^{3′}, (CH₂)_{a′}NR^{2′}COR^{4′}, (CH₂)_{a′}NR^{2′}CO₂R^{5′}, (CH₂)_{a′}NR^{2′}SO₂R^{6′}, (CH₂)_{a′}CONR^{7′}R^{8′}, hydroxyC₁₋₆alkyl, C₁₋₄alkoxyalkyl (optionally substituted by a C₁₋₄alkoxy or hydroxy group), (CH₂)_{a′}CO₂C₁₋₆alkyl, (CH₂)_{b′}OC(O)R^{9′}, CR^{10′}═NOR^{11′}, CNR^{10′}═NOR^{11′}, COR^{12′}, CONR^{7′}R^{8′}, CONR^{7′}(CH₂)_{c′}OC₁₋₄alkyl, CONR^{7′}(CH₂)_{a′}CO₂R^{13′}, CONHNR^{14′}R^{15′}, CONR^{7′}SO₂R^{16′}, CO₂R^{17′}, cyano, trifluoromethyl, NR^{2′}R^{3′}, NR^{2′}COR^{4′}, NR^{18′}CO(CH₂)_{a′}NR^{18′})R^{19′}, NR^{18′}CONR^{18′}R^{19′}, NR^{2′}CO₂R^{5′}, NR^{2′}SO₂R^{6′}, N═CNR^{18′}NR^{18′}R^{19′}, nitro, hydroxy, C₁₋₆alkoxy, OCF₃, hydroxy C₁₋₆alkoxy, C₁₋₆alkoxyC₁₋₆alkoxy, OC(O)NR^{20′}R^{21′}, SR^{22′}, SOR^{23′}, SO₂R^{23′}, SO₂NR^{20′}R^{21′} or halogen, or R^{1′} is a 5- to 7-membered ring containing 1 to 4 heteroatoms selected from nitrogen, oxygen, or sulfur, optionally substituted with hydrogen, C₁₋₆alkyl, C₃₋₇cycloalkyl, C₃₋₆cycloalkenyl, hydroxyC₁₋₆alkyl, (C₁₋₆alkyl)C₁₋₆alkyl, CONR^{7′}R^{8′}, CO₂R^{17′}, cyano, aryl, trifluoromethyl, nitro, hydroxy, C₁₋₆alkoxy, acyloxy, or halogen;

a′ is 1, 2, 3 or 4;

b′ is 0, 1, 2 or 3;

c′ is 1, 2 or 3;

R^{2′} and R^{3′} are independently hydrogen or C₁₋₆alkyl, or R^{2′} and R^{3′} together with the nitrogen to which they are attached, form a 5- to 6-membered heterocyclic ring which ring may be optionally substituted by an oxo group, or, when there are 6 ring members, the ring may optionally contain one oxygen or one sulfur atom;

R^{4′} is hydrogen, C₁₋₆alkyl or C₁₋₄alkoxyalkyl, or, when R^{1′} is NR^{2′}COR^{4′}, R^{4′} is (CH₂)₁₋₃ and forms a ring with A′;

R^{5′} is C₁₋₆alkyl;

R^{6′} is C₁₋₆alkyl or phenyl;

R^{7′} and R^{8′} are independently hydrogen or C₁₋₆alkyl, or R^{7′} and R^{8′} together with the nitrogen to which they are attached form a 5- to 6-membered saturated heterocyclic ring, wherein when there are 6 ring members, the ring may optionally contain one oxygen or one sulfur atom;

R^{9′} is C₁₋₄alkyl, optionally substituted by a C₁₋₆alkoxy;

R^{10′} and R^{11′} are independently hydrogen or C₁₋₆alkyl;

R^{12′} is hydrogen or C₁₋₆alkyl;

R^{13′} is hydrogen or C₁₋₆alkyl;

R^{14′} and R^{15′} are independently hydrogen or C₁₋₆alkyl;

R^{16′} is hydrogen or C₁₋₆alkyl;

R^{17′} is hydrogen or C₁₋₆alkyl optionally substituted with one or more substituents selected from C₁₋₆alkyl, C₁₋₆alkoxy, hydroxy, or NR^{2′}R^{3′},

R^{18′} and R^{19′} are independently hydrogen or C₁₋₆alkyl;

R^{20′} and R^{21′} are independently hydrogen or C₁₋₆alkyl, or R^{20′} and R^{21′} together with the nitrogen to which they are attached form a 5- to 6-membered saturated heterocyclic ring which, when the ring is 6-membered, may optionally contain in the ring one oxygen or one sulfur atom.

R^{22′} is hydrogen or C₁₋₆alkyl;

R^{23′} is C₁₋₆alkyl;

D′ is either a bond or represents [C(R^{24′})₂]_{a″}, [C(R^{24′})₂]_{a″}CO, CO, SO₂, CO[C(R^{24′})₂]_{a″}, O[C(R^{24′})₂]_{a″}, S[C(R^{24′})₂]_{a″}, O[C(R^{24′})₂]_{a″}CO, [C(R^{24′})₂]_{c″}OCO, NR^{25′}[C(R^{24′})₂]_{a″}, NR^{25′}[C(R^{24′})₂]_{a″}CO, [C(R^{24′})₂]_{c″}NR^{25′}CO, NR^{25″}CO[C(R²⁴)₂]_{a″}, NR^{25′}SO₂[C(R^{24′})₂]_{a″}, [C(R^{24′})₂]_{c″}NR^{25′}SO₂, CR^{24′}═CR^{24′}CO, C≡CCO, (C(R^{24′})₂)_{c″}SO₂, SO₂[C(R^{24′})₂]_{a″}, NR^{25′}[C(R^{24′})₂]_{a″}SO₂, NR^{25′}SO₂[C(R^{24′})₂]_{a″}SO₂, O[C(R^{24′})₂]_{a″}SO₂, SO₂NR^{25′}[C(R^{24′})₂]₁₋₂, [C(R^{24′})₂]_{b″}COO[C(R^{24′})₂]₂, [C(R^{24′})₂]_{b″}CONR^{25′}[C(R^{24′})₂]₁₋₂; and when E′ and G′ together are CR^{27′}—C(R^{26′})₂, then D′ may further be O, NR^{25′}, CONR^{25′}; SO₂NR^{25′}, OCONR^{25′}, NR^{25′}COO, NR^{25′}CONR^{25′}, [C(R^{24′})₂]_{a″}NR^{25′}[C(R^{24′})₂]_{b″}, [C(R^{24′})₂]_{a″}O[C(R^{24′})₂]_{b″}, CO[C(R^{24′})₂]_{a″}NR^{25′}, NR^{25′}[C(R^{24′})₂]_{a″}O, NR^{25′}[C(R^{24′})₂]_{a″}NR^{25′}, O[C(R^{24′})₂)]_{a″}NR^{25′}, O[C(R^{24′})₂]_{a″}O, CO[C(R^{24′})₂]_{a″}O, SO₂[C(R^{24′})₂]_{a″}NR^{25′}, SO₂[C(R^{24′})₂]_{a″}O, [C(R^{24′})₂]_{a″}SO₂NR^{25′}, [C(R^{24′})₂]_{a″}CONR^{25′}, O[C(R^{24′})₂]_{a″}SO₂NR^{25′}, O[C(R^{24′})₂]_{a″}CONR^{25′}, NR^{25′}[C(R^{24′})₂]_{a″}SO₂NR^{25′}, NR^{25′}[C(R^{24′})₂]_{a″}CONR^{25′}, NR^{25′}CO[C(R^{24′})₂]_{a″}NR^{25′}, NR^{25′}SO₂[C(R^{24′})₂]_{a″}NR^{25′}, (C(R^{24′})₂)_{a″}S(C(R^{24′})₂)_{b″}, COO, CR^{24′}OH, C(R^{24′})_{a″}CR^{24′}OH; and when E′ and G′ together are CR^{27′}—C(R^{26′})₂ or C═CR^{26′}, D′ may further be CR^{24′}═CR^{24′} or C≡C; and a″ is 1-6, b″ is 0-1, c″ is 0-2;

R^{24′} is hydrogen or C₁₋₆alkyl;

R^{25′} is hydrogen or C₁₋₆alkyl;

E′ and G′ together are NC(R^{26′})₂, NC(R^{26′})₂C(R^{26′})₂, CR^{27′}C(R^{26′})₂ or C═CR^{26′};

R^{26′} is hydrogen or C₁₋₆alkyl;

R^{27′} is hydrogen, OR^{28′}, NHR^{28′}, CN, NO₂, R^{28′}, SR^{29′}, COR^{28′}, CHOHR^{28′}, CO₂R^{28′}, NHCOR^{28′}, NHCO₂R^{29′}, NHSO₂R^{29′}, or OCONHR^{28′};

R^{28′} is hydrogen, C₁₋₅alkyl, aryl or aralkyl;

R^{29′} is C₁₋₅alkyl, aryl or aralkyl;

R′ is one or more of hydrogen or C₁₋₆alkyl, or R′ is oxo;

J′ is CO or SO₂;

L′ is NR^{30′}, O or C(R^{30′})₂;

R^{30′} is hydrogen or C₁₋₆alkyl;

E represents a group (a):

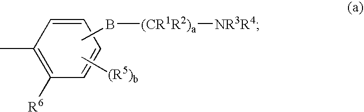

in which

B is oxygen, C≡C, S(O)_{c}, CR⁷═CR⁸, or CR⁷R⁸, or B is NR⁹;

R¹ and R² are independently hydrogen or C₁₋₆alkyl; alternatively B(CR¹R²)ₐ is OCR¹R²CR¹(OH)CR¹R² or OCR¹R²CR¹(OCOCH₃)CR¹R²;

R³ and R⁴ are independently hydrogen, C₁₋₆alkyl, C₃₋₇cycloalkyl, aralkyl, or together with the nitrogen atom to which they are attached form an optionally substituted 5- to 7-membered heterocyclic ring which may contain an additional heteroatom selected from oxygen, nitrogen or sulfur, where optional substituents include C₁₋₆alkyl, aryl, CONR¹⁰R¹¹, NR¹⁰R¹¹, hydroxy, OCOR¹², NHCOCF₃, NHSO₂R¹³, NHCO₂R¹⁴, or NHCOC₀₋₆alkyl wherein the alkyl of NHCOC₀₋₆alkyl is optionally substituted by OH;

R⁵ is hydrogen, C₁₋₆alkyl, aryl, CN, CONR¹⁵R¹⁶, CO₂R¹⁷, trifluoromethyl, NHCO₂R¹⁸, hydroxy, C₁₋₆alkoxy, benzyloxy, OCH₂CO₂C₁₋₆alkyl, OCF₃, S(O)_{d}R¹⁹, SO₂NR²⁰R²¹ or halogen;

R⁶ is hydrogen, C₁₋₆alkyl, aryl, trifluoromethyl, hydroxy, C₁₋₆alkoxy or halogen, or R⁶ taken together with R^{30′} forms a group D where D is (CR²²R²³)ₑ or D is (CR²²R²³)_{f}—G where G is oxygen, sulfur or CR²²═CR²³, CR²²═N, ═CR²²O, ═CR²²S, or ═CR²²—NR²³;

R⁷, R⁸, R¹⁰, R¹¹, R¹², R¹⁵, R¹⁶, R¹⁷, R²⁰, R²¹, R²², and R²³ are independently hydrogen or C₁₋₆alkyl;

R⁹ is hydrogen, C₁₋₆alkyl, or phenylC₁₋₆alkyl;

R¹³, R¹⁴, R¹⁸, and R¹⁹ are independently C₁₋₆alkyl;

a is 1, 2, 3, or 4;

b is 1 or 2;

c and d are independently 0, 1 or 2;

e is 2, 3 or 4;

f is 0, 1, 2 or 3;

alternatively, E represents a group (b):

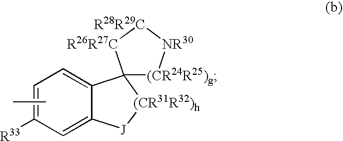

R²⁴, R²⁵, R²⁶, R²⁷, R²⁸, R²⁹, R³¹, and R³² are independently hydrogen or C₁₋₆alkyl;

R³⁰ is hydrogen, C₁₋₆alkyl, or C₃₋₇cycloalkyl;

R³³ is hydrogen, C₁₋₆alkyl, trifluoromethyl, hydroxy or halogen, or R³³ and R^{30′} together form a group -K- where K is (CR³⁴R³⁵)ᵢ or K is (CR³⁴R³⁵)ⱼ—M and M is oxygen, sulfur, CR³⁴═CR³⁵, CR³⁴═N, or N═N;

J is oxygen, CR³⁶R³⁷, or NR³⁸, or J is a group S(O)ₖ;

R³⁴, R³⁵, R³⁶, R³⁷, and R³⁸ are independently hydrogen or C₁₋₆alkyl;

g is 1, 2 or 3;

h is 1, 2 or 3;

i is 2, 3, or 4;

j is 0, 1, 2, or 3;

k is 0, 1 or 2;

alternatively, E represents a group (c):

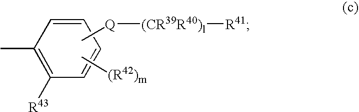

in which:

Q is oxygen, S(O)ₙ, CR⁴⁴═CR⁴⁵, CR⁴⁴R⁴⁵, or Q is NR⁴⁶;

R³⁹ and R⁴⁰ are independently hydrogen or C₁₋₆alkyl;

R⁴¹ is a group of formula (d):

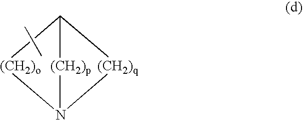

or R⁴¹ is a group of formula (e):

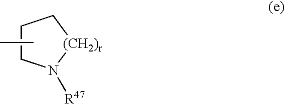

R⁴² is hydrogen, C₁₋₆alkyl, aryl, CN, CONR⁴⁸R⁴⁹, CO₂R⁵⁰, trifluoromethyl, NHCO₂R⁵¹, hydroxy, C₁₋₆alkoxy, benzyloxy, OCH₂CO₂C₁₋₆alkyl, OCF₃, S(O)ₛR⁵², SO₂NR⁵³R⁵⁴, or halogen;

R⁴³ is hydrogen or R⁴³ together with R^{30′} forms a group R where R is CR⁵⁵═CR⁵⁶, CR⁵⁵═CR⁵⁶CR⁵⁵R⁵⁶, or (CR⁵⁵R⁵⁶)ₜ;

R⁴⁴, R⁴⁵, R⁴⁶, R⁴⁸, R⁴⁹, R⁵⁰, R⁵³, R⁵⁴, R⁵⁵, and R⁵⁶ are independently hydrogen or C₁₋₆alkyl;

R⁴⁷ is hydrogen, C₁₋₆alkyl, or C₃₋₇ cycloalkyl;

R⁵¹ and R⁵² are independently C₁₋₆alkyl;

l is 0, 1, 2, or 3;

m is 1 or 2;

n is 0, 1, or 2

o, p, and q are independently integers having the value 1, 2, or 3;

r is 0, 1, 2, or 3;

s is 0, 1, or 2;

t is 2or 3;

alternatively, E represents a group (f):

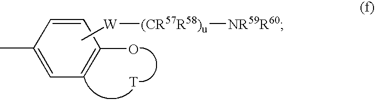

R⁵⁷ and R⁵⁸ are independently hydrogen or C₁₋₆alkyl;

R⁵⁹ and R⁶⁰ are independently hydrogen, C₁₋₆alkyl, C₃₋₇cycloalkyl, aralkyl, or together with the nitrogen atom to which they are attached form an optionally substituted 5- to 7-membered heterocyclic ring which may contain an additional heteroatom selected from oxygen, nitrogen or sulfur, where optional substituents include C₁₋₆alkyl, aryl, CONR⁶¹R⁶², NR⁶¹R⁶², hydroxy, OCOR⁶³, NHCOCF₃, NHSO₂R⁶⁴, NHCO₂R⁶⁵, or NHCOC₀₋₆alkyl wherein the alkyl of NHCOC₀₋₆alkyl is optionally substituted by OH;

T is —(CR⁶⁶R⁶⁷)ᵥ— or —O(CR⁶⁶R⁶⁷)_{w}—;

W is oxygen, S(O)ₓ, NR⁶⁸, or W is CR⁶⁹═CR⁷⁰ or CR⁶⁹R⁷⁰;

R⁶¹, R⁶², R⁶³, R⁶⁶, R⁶⁷ R⁶⁸, R⁶⁹, and R⁷⁰ are independently hydrogen or C₁₋₆alkyl;

R⁶⁴ and R⁶⁵ are independently C₁₋₆alkyl;

u is 1 to 4;

v is 2 or 3;

w is 1, 2, or 3;

x is 0, 1 or 2;

alternatively, E represents a group (g):

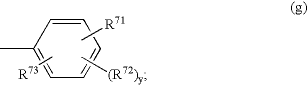

R⁷¹ is a 5- to 7-membered saturated or partially saturated heterocyclic ring containing a nitrogen atom and optionally a further 1 or 2 heteroatoms selected from nitrogen, oxygen or sulfur or R⁷¹ is an optionally substituted 6,6 or 6,5 bicyclic ring containing a nitrogen atom and optionally a further heteroatom selected from oxygen, nitrogen or sulfur, which ring systems may be optionally substituted with one or more of C₁₋₆alkyl and optionally substituted on nitrogen with hydrogen, C₁₋₆alkyl or C₃₋₇cycloalkyl;

R⁷² is hydrogen, C₁₋₆alkyl, aryl, CN, CONR⁷⁴R⁷⁵, CO₂R⁷⁶, trifluoromethyl, NHCO₂R⁷⁷, hydroxy, C₁₋₆alkoxy, benzyloxy, OCH₂CO₂C₁₋₆alkyl, OCF₃, S(O)_{z}R⁷⁸, SO₂NR⁷⁹R⁸⁰, or halogen;

R⁷³ is hydrogen, C₁₋₆alkyl, hydroxy, C₁₋₆alkoxy or halogen, or R⁷³ and R^{30′} taken together from a group —X— where X is (CR⁸¹R⁸²)ₐₐ or X is (CR⁸¹R⁸²)_{ab}—Y and Y is oxygen, sulfur or CR⁸¹═CR⁸²;

R⁷⁴, R⁷⁵, R⁷⁶, R⁷⁹, R⁸⁰, R⁸¹, and R⁸² are independently hydrogen or C₁₋₆alkyl;

R⁷⁷ and R⁷⁸ are independently C₁₋₆alkyl;

y is 1 or 2;

z is 0, 1, or 2;

aa is 2, 3 or 4;

ab is 0, 1, 2 or 3;

alternatively, E represents a group (h):

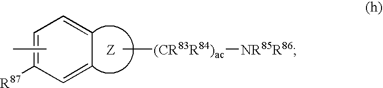

R⁸³ and R⁸⁴ are independently hydrogen or C₁₋₆alkyl;

R⁸⁵ and R⁸⁶ are independently hydrogen, C₁₋₆alkyl, C₃₋₇cycloalkyl, aralkyl, or together with the nitrogen atom to which they are attached form an optionally substituted 5- to 7-membered heterocyclic ring which may contain an additional heteroatom selected from oxygen, nitrogen or sulfur, where optional substituents include C₁₋₆alkyl, aryl, CONR⁸⁸R⁸⁹, NR⁹⁰R⁹¹, hydroxy, OCOR⁹², NHCOCF₃, NHSO₂R⁹³, NHCO₂R⁹⁴, or NHCOC₀₋₆alkyl wherein the alkyl of NHCOC₀₋₆alkyl is optionally substituted by OH;

R⁸⁷ is hydrogen or C₁₋₆alkyl, C₁₋₆alkoxy, or halogen, or R⁸⁷ together with R^{30′} forms a group —AA— where AA is (CR⁹⁵R⁹⁶)_{ad} or AA is (CR⁹⁵═CR⁹⁶)ₐₑ—AB and AB is oxygen, sulfur, CR⁹⁵═CR⁹⁶, CR⁹⁵═N, CR⁹⁵NR⁹⁶ or N═N;

Z is an optionally substituted 5 to 7-membered heterocyclic ring containing 1 to 3 heteroatoms selected from oxygen, nitrogen or sulfur;

R⁸⁸, R⁸⁹, R⁹⁰, R⁹¹, R⁹², R⁹⁵, and R⁹⁶ are independently hydrogen or C₁₋₆alkyl;

R⁹³ and R⁹⁴ are independently C₁₋₆alkyl;

ac is 0 to 4;

ad is 1, 2 or 3;

ae is 0, 1 or 2;

alternatively, E represents a group (i):

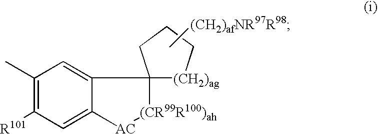

R⁹⁷ and R⁹⁸ are independently hydrogen, C₁₋₆alkyl, C₃₋₇cycloalkyl, aralkyl, or together with the nitrogen atom to which they are attached form an optionally substituted 5- to 7-membered heterocyclic ring which may contain an additional heteroatom selected from oxygen, nitrogen or sulfur, where optional substituents include C₁₋₆alkyl, aryl, CONR¹⁰²R¹⁰³, NR¹⁰⁴R¹⁰⁵, hydroxy, OCOR¹⁰⁶, NHCOCF₃, NHSO₂R¹⁰⁷, NHCO₂R¹⁰⁸, or NHCOC₀₋₆alkyl wherein the alkyl of NHCOC₀₋₆alkyl is optionally substituted by OH;

R⁹⁹ and R¹⁰⁰ are independently hydrogen or C₁₋₆alkyl;

R¹⁰¹ is hydrogen or C₁₋₆alkyl or R¹⁰¹ and R^{30′} together form a group —AD— where AD is (CR¹⁰⁹R¹¹⁰)ₐᵢ or AD is (CR¹⁰⁹R¹¹⁰)ₐⱼ—AE and AE is oxygen, sulfur or CR¹⁰⁹═CR¹¹⁰;

AC is oxygen, CR¹¹¹R¹¹² or NR¹¹³ or AC is a group S(O)ₐₖ;

R¹⁰², R¹⁰³, R¹⁰⁴, R¹⁰⁵, R¹⁰⁶, R¹⁰⁹, R¹¹⁰, R¹¹¹, R¹¹², and R¹¹³ are independently hydrogen or C₁₋₆alkyl;

R¹⁰⁷ and R¹⁰⁸ are independently C₁₋₆alkyl;

af is 0, 1, 2, 3, or 4;

ag is 1, 2, or 3;

ah is 1, 2, 3 or 4;

ai is 2, 3 or 4;

aj is 0, 1, 2, or 3; and

ak is 0, 1 or 2.

For compounds of formula (I) various embodiments are as follows. It will be understood that the basic nitrogen in moiety E may be optionally quaternized with C₁₋₆alkyl or is optionally present as the N-oxide.

Suitably, A′ is aryl or heteroaryl, each of which is optionally substituted with one or more of R^{1′}. Alternatively, A′ is suitably aryl or heteroaryl fused to a saturated or partly unsaturated 5-7-membered ring to form a higher order ring moiety, which ring moiety optionally contains 1 or 2 heteroatoms selected from oxygen, nitrogen or sulfur, wherein nitrogen may be optionally substituted with hydrogen, C₁₋₆alkyl or C₃₋₇cycloalkyl; wherein the higher order ring moiety is optionally substituted with one or more of R^{1′}. Preferably A′ is phenyl, 5,6,7,8-tetrahydro-1-naphthalenyl, 1H-indol-4-yl, or 2-benzothiazolyl.

Suitably, R^{1′} is hydrogen, C₁₋₆alkyl, C₂₋₆alkenyl, C₂₋₆alkynyl, C₃₋₇cycloalkyl, C₃₋₆cycloalkenyl, CH₂CF₃, aryl, aralkyl, (CH₂)_{a′}NR^{2′}R^{3′}, (CH₂)_{a′}NR^{2′}COR^{4′}, (CH₂)ₐNR^{2′}CO₂R^{5′}, (CH₂)_{a′}NR^{2′}SO₂R^{6′}, (CH₂)_{a′}CONR^{7′}R^{8′}, hydroxyC₁₋₆alkyl, C₁₋₄alkoxyalkyl (optionally substituted by a C₁₋₄alkoxy or hydroxy group), (CH₂)_{a′}CO₂C₁₋₆alkyl, (CH₂)_{b′}OC(O)R^{9′}, CR^{10′}═NOR^{11′}, CNR^{10′}═NOR^{11′}, COR^{12′}, CONR^{7′}R^{8′}, CONR^{7′}(CH₂)_{c′}OC₁₋₄alkyl, CONR^{7′}(CH₂)_{a′}CO₂R^{13′}, CONHNR^{14′}R^{15′}, CONR^{7′}SO₂R^{16′}, CO₂R^{17′}, cyano, trifluoromethyl, NR^{2′}R^{3′}, NR^{2′}COR^{4′}, NR^{18′}CO(CH₂)_{a′}NR^{18′}R^{19′}, nitro, NR^{18′}CONR^{18′}R^{19′}, NR^{2′}CO₂R^{5′}, NR^{2′}SO₂R^{6′}, N═CNR^{18′}NR^{18′}R^{19′}, nitro, hydroxy, C₁₋₆alkoxy, OCF₃, hydroxyC₁₋₆alkoxy, C₁₋₆alkoxyC₁₋₆alkoxy, OC(O)NR^{20′}R^{21′}, SR^{22′}, SOR^{23′}, SO₂R^{23′}, SO₂NR^{20′}R^{21′} or halogen, or suitably R^{1′} is a 5- to 7-membered heterocyclic ring containing 1 to 4 heteroatoms selected from oxygen, nitrogen, or sulfur, suitable heterocyclic rings include aromatic groups such as thienyl, furyl, pyrrolyl, triazolyl, diazolyl, imidazolyl, oxazolyl, thiazolyl, oxadiazolyl, isothiazolyl, isoxazolyl, thiadiazolyl, pyridyl, pyrimidyl, pyrazinyl, and dioxanyl. Saturated and partially saturated rings are also within the scope of the invention, in particular rings including an oxo or thioxo moiety such as lactams and thiolactams. Suitably, the heterocyclic ring can be linked to the remainder of the molecule via a carbon atom, or, when present, a nitrogen atom. Suitably these rings may be optionally substituted with one or more of hydrogen, C₁₋₆alkyl, C₃₋₇cycloalkyl, C₃₋₆cycloalkenyl, hydroxyC₁₋₆alkyl, (C₁₋₆alkyl)C₁₋₆alkyl, CONR^{7′}R^{8′}, CO₂R^{17′}, cyano, aryl, trifluoromethyl, nitro, hydroxy, C₁₋₆alkoxy, acyloxy, or halogen. Preferably, R^{1′} is one or more of C₁₋₆alkyl, (CH₂)ₐNR²COR⁴, CF₃, CO₂C₁₋₆alkyl, C₁₋₆alkoxy, halogen, or cyano.

Suitably, a′ is 1, 2, 3 or 4; b′ is 0, 1, 2 or 3; and c′ is 1, 2 or 3.

Suitably, R^{2′} and R^{3′} are independently hydrogen or C₁₋₆alkyl, or suitably, R^{2′} and R^{3′} together with the nitrogen to which they are attached, form a 5- to 6-membered heterocyclic ring. Suitably, the ring may be optionally substituted by an oxo group, or, when R^{2′} and R^{3′} form a 6-membered ring, the ring may optionally contain one oxygen or one sulfur atom. When the ring is a 6-membered ring substituted by an oxygen or sulfur atom, the oxygen or sulfur atom are preferably in the 4-position.

Suitably, R⁴ is hydrogen, C₁₋₆alkyl or C₁₋₄alkoxyalkyl, or, when R^{1′} is NR^{2′}COR^{4′}, R^{4′} is (CH₂)₁₋₃and forms a ring with A′.

Suitably R^{5′} is C₁₋₆alkyl.

Suitably, R^{6′} is C₁₋₆alkyl or phenyl.

Suitably, R^{7′} and R⁸ are independently hydrogen or C₁₋₆alkyl, or suitably, R^{7′} and R^{8′} together with the nitrogen to which they are attached form a 5- to 6-membered saturated heterocyclic ring. Suitably, when the ring is 6-membered, the ring may optionally contain one oxygen or one sulfur atom.

Suitably, R^{9′} is C₁₋₄alkyl, wherein the C₁₋₆alkyl is optionally substituted by a C₁₋₆alkoxy.

Suitably, R^{10′} and R^{11′} are independently hydrogen or C₁₋₆alkyl.

Suitably, R^{12′} is hydrogen or C₁₋₆alkyl.

Suitably, R^{13′} is hydrogen or C₁₋₆alkyl.

Suitably, R^{14′} and R^{15′} are independently hydrogen or C₁₋₆alkyl.

Suitably, R^{16′} is hydrogen or C₁₋₆alkyl.

Suitably, R^{17′} is hydrogen or C₁₋₆alkyl, wherein the C₁₋₆alkyl is optionally substituted with one or more substituents selected from C₁₋₆alkyl, C₁₋₆alkoxy, hydroxy, or NR^{2′}R^{3′}. Preferably, when there is more than one substituent, there are two substituents.

Suitably, R^{18′} and R^{19′} are independently hydrogen or C₁₋₆alkyl.

Suitably, R^{20′} and R^{21′} are independently hydrogen or C₁₋₆alkyl, or suitably, R^{20′} and R^{21′} together with the nitrogen to which they are attached form a 5- to 6-membered saturated heterocyclic ring which, when there are 6 ring members, may optionally contain in the ring one oxygen or one sulfur atom.

Suitably, R^{22′} is hydrogen or C₁₋₆alkyl.

Suitably, R^{23′} is C₁₋₆alkyl.

Suitably, D′ is either a bond or represents [C(R^{24′})₂]_{a″}, [C(R^{24′})₂]_{a″}CO, SO₂, CO, CO[C(R^{24′})₂]_{a″}, O[C(R^{24′})₂]_{a″}, S[C(R^{24′})₂]_{a″}, O[C(R^{24′})₂]_{a″}CO, [C(R^{24′})₂]_{c″}OCO, NR^{25′}[C(R^{24′})₂]_{a″}, NR^{25′}[C(R^{24′})₂]_{a″}CO, [C(R^{24′})₂]_{c″}NR^{25′}CO, NR^{25′}CO[C(R^{24′})₂]_{a″}, NR^{25′}SO₂[C(R^{24′})₂]_{a″}, [C(R^{24′})₂]_{c″}NR^{25′}SO₂, CR^{24′}═CR^{24′}CO, C≡CCO, (C(R^{24′})₂)_{c″}SO₂, SO₂[C(R^{24′})₂]_{a″}, NR^{25′}[C(R^{24′})₂]_{a″}SO₂, NR^{25′}SO₂[C(R^{24′})₂]_{a″}SO₂, O[C(R^{24′})₂]_{a″}SO₂, SO₂NR^{25′}[C(R^{24′})₂]₁₋₂, [C(R^{24′})₂]_{b″}COO[C(R^{24′})₂]₂, [C(R^{24′})₂]_{b″}CONR^{25′}[C(R^{24′})₂]₁₋₂; and when E′ and G′ together are CR^{27′}—C(R^{26′})₂, then D′ may further be O, NR^{25′}, CONR^{25′}, SO₂NR^{25′}, OCONR^{25′}, NR²⁵″COO, NR²⁵CONR^{25′}, [C(R^{24′})₂]_{a″}NR^{25′}[C(R^{24′})₂]_{b″}, [C(R^{24′})₂]_{a″}O[CR^{24′})₂]_{b″}, CO[C(R^{24′})₂]_{a″}NR^{25′}, NR^{25′}[C(R^{24′})₂]_{a″}O, NR^{25′}[C(R^{24′})₂]_{a″}NR^{25′}, O[C(R^{24′})₂)]_{a″}NR^{25′}, O[C(R^{24′})₂]_{a″}O, CO[CR^{24′})₂]_{a″}O, SO₂[C(R^{24′})₂]_{a″}NR^{25′}, SO₂[C(R^{24′})₂]_{a″}O, [C(R^{24′})₂]_{a″}SO₂NR^{25′}, [C(R^{24′})₂]_{a″}CONR^{25′}, O[C(R^{24′})₂]_{a″}SO₂NR^{25′}, O[C(R^{24′})₂]_{a″}CONR^{25′}, NR^{25′}[CR^{24′})₂]_{a″}SO₂NR^{25′}, NR^{25′}[C(R^{24′})₂]_{a″}CONR^{25′}, NR^{25′}CO[C(R^{24′})₂]_{a″}NR^{25′}, NR^{25′}SO₂[C(R^{24′})₂]_{a″}NR^{25′}, (C(R^{24′})₂)_{a″}S(C(R^{24′})₂)_{b″}, COO, CR^{24′}OH, C(R^{24′})_{a″}CR^{24′}OH; and when E′ and G′ together are CR^{27′}—C(R^{26′})₂ or C═CR^{26′}, D′ may further be CR²⁴═CR²⁴ or C≡C; and a″ is 1-6, b″ is 0-1, c″ is 0-2. Preferably, D′ is a bond, CO or SO₂.

Suitably, R^{24′} is hydrogen or C₁₋₆alkyl.

Suitably, R^{25′} is hydrogen or C₁₋₆alkyl.

Suitably, E′ and G′ together are NC(R^{26′})₂, NC(R^{26′})₂C(R^{26′})₂, CR^{27′}C(R^{26′})₂ or C═CR^{26′}. Preferably, E′ and G′ together are NC(R²⁶)₂.

Suitably, R^{26′} is hydrogen or C₁₋₆alkyl. Preferably, R^{26′} is hydrogen.

Suitably, R^{27′} is hydrogen, OR^{28′}, NHR^{28′}, CN, NO₂, R^{28′}, SR^{29′}, COR^{29′}, CHOHR^{29′}, CO₂R^{29′}, NHCOR^{29′}NHCO₂R^{29′}, NHSO₂R^{29′}, or OCONHR^{29′},

Suitably, R^{28′} is hydrogen, C₁₋₅alkyl, aryl or aralkyl.

Suitably, R^{29′} is C₁₋₅alkyl, aryl or aralkyl.

Suitably, R′ is one or more of hydrogen or C₁₋₆alkyl, or R′ is oxo. Preferably, R′ is hydrogen.

Suitably, J′ is CO or SO₂. Preferably, J′ is CO.

Suitably, L′ is NR^{30′}, O, or C(R^{30′})₂. Preferably, L′ is NR^{30′}.

Suitably, R^{30′} is hydrogen or C₁₋₆allyl. Preferably, R^{30′} is hydrogen.

Suitably, substituent E is selected from the following groups:

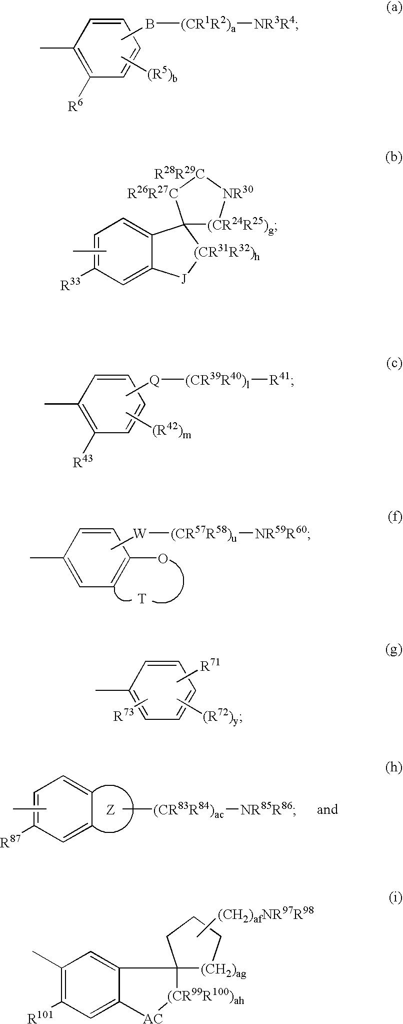

E suitably represents a group (a):

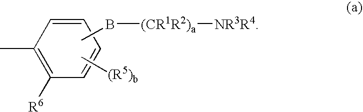

B is suitably oxygen, C≡C, S(O)_{c}, CR⁷═CR⁸, or CR⁷R⁸, or B is NR⁹. B is preferably CR⁷R⁸, or oxygen.

R¹ and R² are suitably independently hydrogen or C₁₋₆alkyl. Preferably, R¹ and R² are each hydrogen. Alternatively, B(CR¹R²)ₐ is OCR¹R²CR¹(OH)CR¹R² or OCR¹R²CR¹(OCOCH₃)CR¹R². Preferably, when B(CR¹R²)ₐ is OCR¹R²CR¹(OH)CR¹R² or OCR¹R²CR¹(OCOCH₃)CR¹R², R¹ and R² are hydrogen.

R³ and R⁴ are suitably independently hydrogen, C₁₋₆alkyl, C₃₋₇cycloalkyl, aralkyl, or together with the nitrogen atom to which they are attached form an optionally substituted 5- to 7-membered heterocyclic ring which may contain an additional heteroatom selected from oxygen, nitrogen or sulfur, where optional substituents include C₁₋₆alkyl, aryl, CONR¹⁰R¹¹, NR¹⁰R¹¹, hydroxy, OCOR¹², NHCOCF₃, NHSO₂R¹³, NHCO₂R¹⁴, or NHCOC₀₋₆alkyl wherein the alkyl of NHCOC₀₋₆alkyl is optionally substituted by OH. Preferably R³ and R⁴ are independently C₁₋₆alkyl, C₃₋₇cycloalkyl, or together with the nitrogen atom to which they are attached form an optionally substituted 5- to 7-membered heterocyclic ring which may contain an additional heteroatom selected from oxygen, nitrogen or sulfur.

Preferably, B—(CR¹R²)ₐ—NR³R⁴ is ortho to R⁵, meta to L′ and para to R⁶, and R⁵ is para to L′.

R⁵ is suitably hydrogen, C₁₋₆alkyl, aryl, CN, CONR¹⁵R¹⁶, CO₂R¹⁷, trifluoromethyl, NHCO₂R¹⁸, hydroxy, C₁₋₆alkoxy, benzyloxy, OCH₂CO₂C₁₋₆alkyl, OCF₃, S(O)_{d}R¹⁹, SO₂NR²⁰R²¹, or halogen. R⁵ is preferably C₁₋₆alkoxy, SC₁₋₆alkyl or halogen.

R⁶ is suitably hydrogen, C₁₋₆alkyl, aryl, trifluoromethyl, hydroxy, C₁₋₆alkoxy, or halogen, or R⁶ taken together with R′^{30′} forms a group D where D is (CR²²R²³)ₑ or D is (CR²²R²³)_{f}—G where G is oxygen, sulfur, or CR²²═CR²³, CR²²═N, ═CR²²O, ═CR²²S, or ═CR²²—NR²³. Preferably, R⁶ is hydrogen.

R⁷, R⁸, R¹⁰, R¹¹, R¹², R¹⁵, R¹⁶, R¹⁷, R²⁰, R²¹, R²², and R²³ are suitably independently hydrogen or C₁₋₆alkyl.

R⁹ is suitably hydrogen, C₁₋₆alkyl, or phenylC₁₋₆alkyl.

R¹³, R¹⁴, R¹⁸, and R¹⁹ are suitably independently C₁₋₆alkyl.

a is suitably 1, 2, 3, or 4. Preferably, a is 2 or 3.

b is suitably 1 or 2. Preferably, b is 1.

c and d are suitably independently 0, 1, or 2.

e is suitably 2, 3, or 4.

f is suitably 0, 1, 2, or 3.

Alternatively, E suitably represents a group (b):

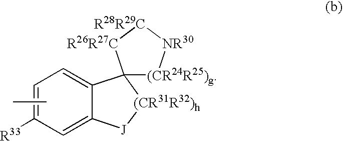

R²⁴, R²⁵, R²⁶, R²⁷, R²⁸, R²⁹, R³¹, and R³² are suitably independently hydrogen or C₁₋₆alkyl. R²⁴, R²⁵, R²⁶, R²⁷, R²⁸, R²⁹, R³¹, and R³² are preferably hydrogen.

R³⁰ is suitably hydrogen, C₁₋₆alkyl, or C₃₋₇cycloalkyl. Preferably, R³⁰ is C₁₋₆alkyl or C₃₋₇cycloalkyl.

R³³ is suitably hydrogen, C₁₋₆alkyl, trifluoromethyl, hydroxy or halogen, or R³³ and R^{30′} together form a group -K- where K is (CR³⁴R³⁵)ᵢ or K is (CR³⁴R³⁵)ⱼ—M and M is oxygen, sulfur, CR³⁴═CR³⁵, CR³⁴═N, or N═N. Preferably, R³³ is hydrogen.

J is suitably oxygen, CR³⁶R³⁷, or NR³⁸, or J is a group S(O)ₖ. Preferably, 3 is oxygen. Preferably, J is para to L′.

R³⁴, R³⁵, R³⁶, R³⁷, R³⁸ are suitably independently hydrogen or C₁₋₆alkyl.

g is suitably 1, 2, or 3. Preferably, g is 2 or 3.

h is suitably 1, 2, or 3. Preferably, h is 1.

i is suitably 2, 3, or 4.

j is suitably 0, 1, 2, or 3.

k is suitably 0, 1 or 2.

Alternatively, E suitably represents a group (c):

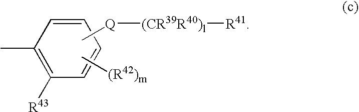

Suitably, Q is oxygen, S(O)ₙ, CR⁴⁴═CR⁴⁵, C═C, or CR⁴⁴R⁴⁵, wherein n is 0, 1 or 2, and R⁴⁴ and R⁴⁵ are independently hydrogen or C₁₋₆alkyl, or suitably, Q is NR⁴⁶ wherein R⁴⁶ is hydrogen or alkyl; suitably, R³⁹ and R⁴⁰ are independently hydrogen or C₁₋₆alkyl; suitably, R⁴² is hydrogen, C₁₋₆alkyl, aryl, CN, CONR⁴⁸R⁴⁹, CO₂R⁵⁰, trifluoromethyl, NHCO₂R⁵¹, hydroxy, C₁₋₆alkoxy, benzyloxy, OCH₂CO₂C₁₋₆allyl, OCF₃, S(O)ₛR⁵², SO₂NR⁵³R⁵⁴, or halogen, wherein R⁴⁸, R⁴⁹, R⁵⁰, R⁵³, and R⁵⁴ are hydrogen or C₁₋₆alkyl, and R⁵¹ and R⁵² are C₁₋₆alkyl; suitably, R⁴³ is hydrogen or R⁴³ together with R^{30′} forms a group R where R is CR⁵⁵═CR⁵⁶, CR⁵⁵═CR⁵⁶CR⁵⁵R⁵⁶, or (CR⁵⁵R⁵⁶)ₜ wherein

R⁵⁵ and R⁵⁶ are independently hydrogen or C₁₋₆alkyl and t is 2 or 3; suitably, R⁴¹ is selected from a group of formula (d) or (e); suitably R⁴⁷ is hydrogen C₁₋₆alkyl, or C₃₋₇ cycloalkyl; suitably, l is 0, 1, 2 or 3, m is 1 or 2, n and s are independently 0, 1 or 2, o, p and q are independently 1, 2 or 3, and r is 0, 1, 2 or 3.

Alternatively, E suitably represents a group (f):

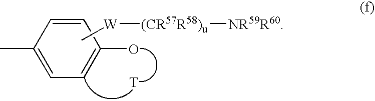

Suitably, R⁵⁷ and R⁵⁸ are independently hydrogen or C₁₋₆alkyl; suitably R⁵⁹ and R⁶⁰ are independently hydrogen, C₁₋₆alkyl, C₃₋₇cycloalkyl, aralkyl, or together with the nitrogen atom to which they are attached form an optionally substituted 5- to 7-membered heterocyclic ring which may contain an additional heteroatom selected from oxygen, nitrogen or sulfur, where optional substituents include C₁₋₆alkyl, aryl, CONR⁶¹R⁶², NR⁶¹R⁶², hydroxy, OCOR⁶³, NHCOCF₃, NHSO₂R⁶⁴, NHCO₂R⁶⁵ or NHCOC₀₋₆alkyl wherein the alkyl of NHCOC₀₋₆alkyl is optionally substituted by OH, and wherein R⁶¹, R⁶², and R⁶³ are independently hydrogen or C₁₋₆alkyl, and R⁶⁴ and R⁶⁵ are independently C₁₋₆alkyl; suitably, T is —(CR⁶⁶R⁶⁷)ᵥ— or —O(CR⁶⁶R⁶⁷)_{w}—, wherein R⁶⁶ and R⁶⁷ are independently hydrogen or C₁₋₆alkyl, wherein v is 2 or 3, and w is 1, 2 or 3; suitably, W is oxygen, S(O)ₓ, wherein x is 0, 1 or 2, or W is NR⁶⁸, wherein R⁶⁸ is hydrogen or C₁₋₆alkyl, or W is CR⁶⁹═CR⁷⁰, C═C, or CR⁶⁹R⁷⁰, wherein R⁶⁹ and R⁷⁰ are independently hydrogen or C₁₋₆alkyl; and suitably, u is an integer from 1-4.

Alternatively, E suitably represents a group (g):

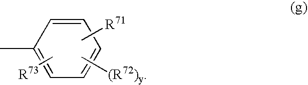

Suitably, R⁷¹ is an optionally substituted 5- to 7-membered saturated or partially saturated heterocyclic ring containing a nitrogen atom and optionally a further one or two heteroatoms selected from nitrogen, oxygen or sulfur, or R⁷¹ is an optionally substituted 6,6 or 6,5-bicyclic ring system containing a nitrogen atom and optionally a further heteroatom selected from oxygen, nitrogen or sulfur, which ring systems may be optionally substituted with one or more of C₁₋₆alkyl, and substituted on nitrogen with hydrogen, C₁₋₆alkyl, or C₃₋₇cycloalkyl. Examples of such ring systems include, but are not limited to, pyrrolidine, piperidine, piperazine, morpholine, imidazolidine, pyrazolidine, 1,2,3,6-tetrahydropyridine, hexahydroazepine, tropane, isoquinuclidine and granatane rings. Preferably, R⁷¹ is an optionally substituted 5- or 6-membered saturated or partially saturated heterocyclic ring containing a nitrogen atom and substituted on nitrogen with C₁₋₆alkyl or C₃₋₇cycloalkyl.

R⁷¹ is preferably located meta to L′, ortho to R⁷² and para to R⁷³, and R⁷² is located para to L′.

Suitably, R⁷² is hydrogen, C₁₋₆alkyl, aryl, CN, CONR⁷⁴R⁷⁵, CO₂R⁷⁶, trifluoromethyl, NHCO₂R⁷⁷, hydroxy, C₁₋₆alkoxy, benzyloxy, OCH₂CO₂C₁₋₆alkyl, OCF₃, S(O)_{z}R⁷⁸, SO₂NR⁷⁹R⁸⁰, or halogen wherein R⁷⁴, R⁷⁵, R⁷⁶, R⁷⁹ and R⁸⁰ are independently hydrogen or C₁₋₆alkyl, R⁷⁷ and R⁷⁸ are C₁₋₆alkyl, and z is 0, 1, or 2. R⁷² is preferably C₁₋₆alkoxy, SC₁₋₆alkyl or halogen.

R⁷³ is hydrogen, C₁₋₆alkyl, hydroxy, C₁₋₆alkoxy or halogen, or R⁷³ and R^{30′} taken together from a group —X— where X is (CR⁸¹R⁸²)ₐₐ, wherein aa is 2, 3 or 4, and R⁸¹ and R⁸² are independently hydrogen or C₁₋₆alkyl, or X is (CR⁸¹R⁸²)_{ab}—Y, wherein ab is 0, 1, 2 or 3, and Y is oxygen, sulfur or CR⁸¹═CR⁸² wherein R⁸¹ and R⁸² are independently hydrogen or C₁₋₆alkyl. Preferably, R⁷³ is hydrogen.

Suitably, y is an integer from 1-2. Preferably, y is 1.

Alternatively, E suitably represents a group (h):

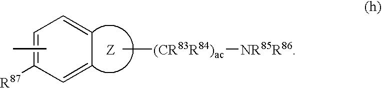

Suitably, R⁸⁷ is hydrogen, C₁₋₆alkyl, C₁₋₆alkoxy or halogen, or R⁸⁷ together with R^{30′} form a group —AA—, wherein AA is (CR⁹⁵R⁸⁸)ad, wherein ad is 1, 2 or 3, and R⁹⁵ and R⁸⁸ are independently hydrogen or C₁₋₆alkyl, or AA is (CR⁹⁵CR⁹⁶)ₐₑ—AB, wherein ae is 0, 1 or 2, and AB is oxygen, sulfur, CR⁹⁵═CR⁹⁶, CR⁹⁵═N, CR⁹⁵NR⁹⁶ or N═N wherein R⁹⁵ and R⁹⁶ are independently hydrogen or C₁₋₆alkyl; suitably, R⁸³ and R⁸⁴ are independently hydrogen or C₁₋₆alkyl; suitably, R⁸⁵ and R⁸⁶ are independently hydrogen, C₁₋₆alkyl, C₃₋₇cycloalkyl, aralkyl, or together with the nitrogen atom to which they are attached form an optionally substituted 5- to 7-membered heterocyclic ring which may contain an additional heteroatom selected from oxygen, nitrogen or sulfur, where optional substituents include C₁₋₆alkyl, aryl, CONR⁸⁸R⁸⁹, NR⁹⁰R⁹¹, hydroxy, OCOR⁹², NHCOCF₃, NHSO₂R⁹³, NHCO₂R⁹⁴, or NHCOC₀₋₆alkyl wherein the alkyl of the NHCOC₀₋₆alkyl is optionally substituted by OH, and wherein R⁸⁸, R⁸⁹, R⁹⁰, R⁹¹ and R⁹² are independently hydrogen or C₁₋₆alkyl, and R⁹³ and R⁹⁴ are independently C₁₋₆alkyl; suitably Z is an optionally substituted 5 to 7-membered heterocyclic ring containing 1 to 3 heteroatoms selected from oxygen, nitrogen or sulfur; suitably ac is 0-4.

Alternatively, E suitably represents a group (i):

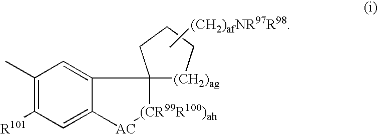

Suitably, R¹⁰¹ is hydrogen or C₁₋₆alkyl or R¹⁰¹ and R^{30′} together form a group —AD— wherein AD is (CR¹⁰⁹R¹¹⁰)ai wherein ai is 2, 3 or 4 or AD is (CR¹⁰⁹R¹¹⁰)ₐⱼ—AE wherein aj is 0, 1, 2 or 3 and AE is oxygen, sulfur or CR¹⁰⁹═CR¹¹⁰, and R¹⁰⁹ and R¹¹⁰ are independently hydrogen or C₁₋₆alkyl; suitably, R⁹⁷ and R⁹⁸ are independently hydrogen, C₁₋₆alkyl, C₃₋₇cycloalkyl, aralkyl, or together with the nitrogen atom to which they are attached form an optionally substituted 5- to 7-membered heterocyclic ring which may contain an additional heteroatom selected from oxygen, nitrogen or sulfur, where optional substituents include C₁₋₆alkyl, aryl, CONR¹⁰²R¹⁰³, NR¹⁰⁴R¹⁰⁵, hydroxy, OCOR¹⁰⁶, NHCOCF₃, NHSO₂R¹⁰⁷, NHCO₂R¹⁰⁸, or NHCOC₀₋₆alkyl wherein the alkyl of NHCOC₀₋₆alkyl is optionally substituted by OH, and wherein R¹⁰², R¹⁰³, R¹⁰⁴, R¹⁰⁵ and R¹⁰⁶ are independently hydrogen or C₁₋₆alkyl, and R¹⁰⁷ and R¹⁰⁸ are independently C₁₋₆alkyl; suitably, R⁹⁹ and R¹⁰⁰ are independently hydrogen or C₁₋₆alkyl; suitably, AC is oxygen, CR¹¹¹R¹¹² or NR¹¹³ wherein R¹¹¹, R¹¹² and R¹¹³ are independently hydrogen or C₁₋₆alkyl or AC is a group S(O)ak wherein ak is 0, 1 or 2; suitably, ag is an integer from 1-3, ah is an integer from 1-4, and af is 0-4.

Suitably, A′ is phenyl, 5,6,7,8-tetrahydro-1-naphthalenyl, 1H-indol-4-yl, or 2-benzothiazolyl, R^{1′} is one or more of C₁₋₆alkyl, (CH₂)ₐNR²COR⁴, CF₃, CO₂C₁₋₆alkyl, C₁₋₆alkoxy, halogen, or cyano, D′ is a bond, E′ and G′ together are NC(R²⁶)₂, R′ is hydrogen, J′ is CO, L′ is NR³⁰, and E is group (a), (b), (c), (f), (g), (h), or (i).

More preferably, A′ is phenyl, 5,6,7,8-tetrahydro-1-napthalenyl, 1H-indol-4-yl, or 6-chloro-2-benzothiazolyl; and when A′ is phenyl, R^{1′} is one or more of C₁₋₆alkyl, CF₃, CO₂CH₂CH₃, C₁₋₆alkoxy, halogen, or cyano substituted at the 2,3-, 2,4-, 2,5-, 2-, 3-, 4-, 3,4-, and 3,5-positions, D′ is a bond, E′ and G′ together are NCH₂, R′ is hydrogen, J′ is CO, L′ is NH, and E is a group (a), (b), or (g).

Preferably, E is selected from group (a), (b) and (g).

More preferably, when E is group (a), L′ is attached to group (a) meta to B—(CR¹R²)ₐ—NR³R⁴ and para to (R⁵)_{b}, wherein B is oxygen or CR⁷R⁸, R¹ and R² are hydrogen, R⁵ is methoxy, methylthio or iodo, R³ and R⁴ are independently C₃₋₆alkyl, or R³ and R⁴ taken together with the nitrogen to which they are attached form a 5- or 6-membered heterocyclic ring optionally substituted with one or more of C₁₋₆alkyl and acetamido or hydroxyl, R⁶ is hydrogen, a is 2 or 3 when B is oxygen and a is 2 when B is CR⁷R⁸, and b is 1.

Most preferably, when E is group (a), L′ is attached to group (a) meta to B—(CR¹R²)ₐ—NR³R⁴ and para to (R⁵)_{b}, wherein B is oxygen or CH₂, R¹ and R² are hydrogen, R⁵ is methoxy, R³ and R⁴ are independently isopropyl or tert-butyl, or R³ and R⁴ taken together with the nitrogen to which they are attached are 1-(2,2,6,6-tetramethylpiperidinyl), 1-(4acetamido-2,2,6,6-tetramethyl piperidinyl), 1-(4-hydroxy-2,2,6,6-tetramethyl piperidinyl) or 1-(4-hydroxy-2,2,4,6,6-pentamethyl piperidinyl), R⁶ is hydrogen, a is 2 when B is oxygen, and b is 1.

More preferably, when E is group (b), L′ is attached to group (b) para to J, J is oxygen, R³³ is hydrogen, R²⁴, R²⁵, R²⁶, R²⁷, R²⁸, R²⁹, R³¹ and R³² are hydrogen, R³⁰ is C₃₋₆alkyl, g is 2 and h is 1.

Most preferably, when E is group (b), L′ is attached to group (b) para to J, J is oxygen, R³³ is hydrogen, R²⁴, R²⁵, R²⁶, R²⁷, R²⁸, R²⁹, R³¹ and R³² are hydrogen, R³⁰ is isopropyl, g is 2, and h is 1.

More preferably, when E is group (g), L′ is attached to group (g) meta to R⁷¹ and para to R⁷², R⁷¹ is an optionally substituted 5- or 6-membered saturated or partially saturated heterocyclic ring containing a nitrogen atom substituted on nitrogen with C₃₋₆alkyl or C₃₋₇cycloalkyl, R⁷² is methoxy, methylthio or iodo, y is 1, and R⁷³ is hydrogen.

Most preferably, when E is group (g), L′ is attached to group (g) meta to R⁷¹ and para to R⁷² wherein R⁷¹ is piperidin-4-yl substituted on nitrogen with isopropyl, R⁷² is methoxy, y is 1, and R⁷³ is hydrogen.

A particularly effective subgenus of compounds of formula (I) is wherein, A′ is phenyl, 5,6,7,8-tetrahydro-1-naphthalenyl, or 1H-indol-4yl; and when A′ is phenyl, R^{1′} is methyl, chloro or trifluoromethyl substituted at the 2 and/or 3-positions, or R^{1′} is 2,4-dimethyl, 2-methoxy-5-chloro, 2-methyl, 3-ethoxycarbonyl, or 3,5-dichloro, D′ is a bond, E′ and G′ together are NCH₂, R′ is hydrogen, J′ is CO, L′ is NH, and E is group (g).

The term “acyloxy” is used herein at all occurrences to mean a moiety —O—C(O)—R, wherein R is hydrogen or C₁₋₆alkyl as defined below.

The term “C₁₋₄alkanoyl” is used herein at all occurrences to mean a —C(O)C₁₋₄alkyl group wherein the alkyl portion is as defined below.

The term “alkenyl” is used herein at all occurrences to mean a straight or branched chain radical of 2 to 6 carbon atoms, unless the length is limited thereto, wherein there is at least one double bond between two of the carbon atoms in the chain, including, but not limited to, ethenyl, 1-propenyl, 2-propenyl, 2-methyl-1-propenyl, 1-butenyl, 2-butenyl, and the like.

The term “alkoxy” is used herein at all occurrences to mean a straight or branched chain radical of 1 to 6 carbon atoms, unless the chain length is limited thereto, bonded to an oxygen atom, including, but not limited to, methoxy, ethoxy, n-propoxy, isopropoxy, and the like.

The term “C₁₋₆alkoxyC₁₋₆alkoxy” is used herein at all occurrences to mean an alkoxy group as defined above, substituted with an alkoxy group as defined above.

The term “C₁₋₄alkoxyalkyl” is used herein at all occurrences to mean a C₁₋₄alkoxy group as defined above bonded to an alkyl group as defined below, including, but not limited to, —CH₂—CH₂—O—CH₂—CH₂—CH₃ and the like.

The term “C₁₋₆alkyl” is used herein at all occurrences to mean a straight or branched chain radical of 1 to 6 carbon atoms, unless the chain length is limited thereto, including, but not limited to, methyl, ethyl, n-propyl, isopropyl, n-butyl, sec-butyl, isobutyl, tert-butyl, and the like.

The term “alkynyl” is used herein at all occurrences to mean a straight or branched chain radical of 2 to 8 carbon atoms, unless the chain length is limited thereto, wherein there is at least one triple bond between two of the carbon atoms in the chain, including, but not limited to, acetylene, 1-propylene, 2-propylene, and the like.

The term “aralkyl” is used herein at all occurrences to mean an aryl moiety as defined above, which is connected to an alkyl moiety as defined below including, but not limited to, benzyl or phenethyl, and the like.

The term “aryl” is used herein at all occurrences to mean a 6-14-membered substituted or unsubstituted aromatic ring(s) or ring systems which may include bi- or tri-cyclic systems, including, but not limited to, phenyl, naphthalenyl, biphenyl, phenanthryl, anthracenyl, and the like.

The term “6,6 or 6,5 bicyclic ring” is used herein at all occurrences to mean a 6,6 or 6,5-bicyclic ring system containing a nitrogen atom and optionally a further heteroatom selected from nitrogen, oxygen, or sulfur, which ring system may be optionally substituted with C₁₋₆alkyl. Examples of such ring systems include, but are not limited to, tropane, isoquinuclidine and granatane rings.

The term “cycloalkenyl” is used herein at all occurrences to mean cyclic radicals, preferably of 5 to 8 carbons, which have at least one double bond between two of the carbon atoms in the ring, including but not limited to, cyclopentenyl, cyclohexenyl, and the like.

The terms “cycloalkyl” and “cyclic alkyl” are used herein at all occurrences to mean cyclic radicals, preferably comprising 3 to 7 carbon atoms which may be mono- or bicyclo-fused ring systems which may additionally include unsaturation, including, but not limited to, cyclopropyl, cyclopentyl, cyclohexyl, 1,2,3,4-tetrahydronaphthalenyl, and the like.

The terms “halo” or “halogen” are used interchangeably herein at all occurrences to mean radicals derived from the elements chlorine, fluorine, iodine and bromine.

The term “heteroaryl” is used herein at all occurrences to mean a 5-14-membered substituted or unsubstituted aromatic ring(s) or ring systems which may include bi- or tri-cyclic systems, which ring or ring systems contain 1 to 4 heteroatoms selected from nitrogen, oxygen, and sulfur, including, but not limited to, indolyl, benzofuranyl, thianaphthenyl, quinolyl, isoquinolyl, pyrrolyl, furanyl, thienyl, pyridyl, and the like.

The term “hydroxyC₁₋₆alkoxy” is used herein at all occurrences to mean an hydroxyl group bonded to an alkoxy group as defined above including, but not limited to, —O—CH₂—CH(OH)CH₃ and the like.

The terms “hydroxyC₁₋₆alkyl” and “hydroxyalkyl” are used herein interchangeably to mean an hydroxyl group bonded to a C₁₋₆alkyl group as defined above, including, but not limited to, methanol, ethanol, n-propanol, isopropanol, n-butanol, sec-butanol, isobutanol, tert-butanol, and the like.

The term “heterocyclic ring” is used herein at all occurrences to mean a saturated or partially saturated 5-10-membered ring system (unless the cyclic ring system is otherwise limited) in which the ring system contains one to 3 heteroatoms selected from oxygen, sulfur, or nitrogen, which ring system may be optionally substituted with C₁₋₆alkyl. Examples of such rings include, but are not limited to, piperidine, tetrahydropyridine, piperazine, pyrrolidine, morpholine, imidazolidine, pyrazolidine, hexahydroazepine, and the like. When the heterocyclic ring is fused to a phenyl group, as when E is the group (h), the term “heterocyclic ring”, together with the phenyl ring to which it is fused, forms a ring which includes, but is not limited to, dihydro-1,4-benzoxazine and 1,2,3,4-tetrahydroquinoline, which may be optionally substituted by C₁₋₆alkyl or oxo.

The term “heteroatom” is used herein at all occurrences to mean an oxygen atom, a sulfur atom or a nitrogen atom. It will be recognized that when the heteroatom is nitrogen, it may form an NRₐ or NRₐR_{b} moiety, wherein Rₐ and R_{b} are, independently, hydrogen or C₁ to C₆ alkyl, or together with the nitrogen to which they are bound, form a saturated or unsaturated 5-, 6- or 7-membered ring, including, but not limited to, pyrrolidine, piperidine, piperazine, morpholine, pyridine, and the like. It will be recognized that the saturated or unsaturated 5-, 6- or 7-membered ring may optionally have one or more additional heteroatoms in the ring.

The term “optionally substituted” is used herein at all occurrences to mean an optionally substituted 5- to 7-membered heterocyclic ring wherein the optional substituents are one or more of C₁₋₆alkyl.

The term “oxo” is used herein at all occurrences to mean a double bonded oxygen atom attached to a chemical moiety as a substituent.

The term “CCR5 mediated disease state” is used herein at all occurrences to mean any disease state which is mediated (or modulated) by CCR5.

Suitably, pharmaceutically acceptable salts of formula (I) include, but are not limited to, salts with inorganic acids such as hydrochloride, sulfate, phosphate, diphosphate, hydrobromide, and nitrate, or salts with an organic acid such as malate, maleate, fumarate, tartrate, succinate, citrate, acetate, lactate, methanesulfonate, p-toluenesulfonate, palmitate, salicylate, and stearate.

The compounds of the invention can exist in unsolvated as well as solvated forms, including hydrated forms. In general, the solvated forms, with pharmaceutically acceptable solvents such as water, ethanol, and the like, are equivalent to the unsolvated forms for purposes of this invention.

The compounds of the present invention may contain one or more asymmetric carbon atoms and may exist in racemic and optically active forms. The stereocenters may be of any combination of R and S configuration, for example, (R,R), (R,S), (S,S) or (S,R). All of these compounds are within the scope of the present invention.

Among the preferred compounds of the invention are the following compounds:

N-[3-(2-Diisopropylamino)ethoxy-4-methoxyphenyl]-4-phenyl-1,2,3,6-tetrahydropyridine-1-carboxamide;

N-[3-(2-Diisopropylamino)ethoxy-4-methoxyphenyl]-4-phenylpiperazine-1-carboxamide;

N-[3-(2-Diisopropylamino)ethoxy-4-methoxyphenyl]-4-(2-methylphenyl)piperazine-1-carboxamide;

N-[3-(2-Diisopropylamino)ethoxy-4-methoxyphenyl]-4-(2,3-dimethylphenyl)piperazine-1-carboxamide;

N-[3-(2-Diisopropylamino)ethoxy-4-methoxyphenyl]-4-(2,3-dichlorophenyl)piperazine-1-carboxamide;

N-[3-(2-diisopropylamino)ethoxy-4-methoxyphenyl]-4-(2-acetamido-methylphenyl)piperazine-1-carboxamide;

N-[3-(2-Diisopropylamino)ethoxy-4-methoxyphenyl]-4-(3-trifluoromethylphenyl)piperazine-1-carboxamide;

N-[3-(2-diisopropylamino)ethoxy-4-methoxyphenyl]-4-(2-methoxyphenyl)piperazine-1-carboxamide;

N-[3-(2-Diisopropylamino)ethoxy-4-methoxyphenyl]-4-(2-chlorophenyl)piperazine-1-carboxamide;

N-[3-(2-Diisopropylamino)ethoxy-4-methoxyphenyl]-4-(3-chlorophenyl)piperazine-1-carboxamide;

N-[3-(2-Diisopropylamino)ethoxy-4-methoxyphenyl]-4-(4-chlorophenyl)piperazine-1-carboxamide;

N-[3-(2-Diisopropylamino)ethoxy-4-methoxyphenyl]-4-(2,6-dimethylphenyl)piperazine-1-carboxamide;

N-[3-(2-Diisopropylamino)ethoxy-4-methoxyphenyl]-4-(3,4-dichlorophenyl)piperazine-1-carboxamide;

N-[3-(2-Diisopropylamino)ethoxy-4-methoxyphenyl]-3-methyl-4-(3-methylphenyl)piperazine-1-carboxamide;

N-[3-(2-Diisopropylamino)ethoxy-4-methoxyphenyl]-4-(4-methoxyphenyl)piperazine-1-carboxamide;

N-[3-(2-Diisopropylamino)ethoxy-4-methoxyphenyl]-4-(2,4-dimethylphenyl)piperazine-1-carboxamide;

N-[3-(2-Diisopropylamino)ethoxy-4-methoxyphenyl]-3-methyl-4-phenylpiperazine-1-carboxamide;

N-[3-(2-Diisopropylamino)ethoxy-4-methoxyphenyl]-4-(3,4-dihydro-2(1H)-quinolinone-6-yl)piperazine-1-carboxamide;

N-[3-(2-Diisopropylamino)ethoxy-4-methoxyphenyl]-4-(3,5-dimethylphenyl)piperazine-1-carboxamide;

N-[3-(2-Diisopropylamino)ethoxy-4-methoxyphenyl]-4-(3-cyanophenyl)piperazine-1-carboxamide;

N-[3-(2-Diisopropylamino)ethoxy-4-methoxyphenyl]-4-[4-(ethoxycarbonyl)phenyl]piperazine-1-carboxamide;

N-[3-(2-Diisopropylamino)ethoxy-4-methoxyphenyl]-4-[2-(ethoxycarbonyl)phenyl]piperazine-1-carboxamide;

N-[2,3-dihydro-1′-isopropyl-spiro[benzofuran-5-yl-3,4′-piperidine]]-4-(2,3-dimethylphenyl)piperazine-1-carboxamide;

N-[3-(2-Diisopropylamino)ethoxy-4-methoxyphenyl]-4-(3-methylphenyl)piperazine-1-carboxamide;

N-[3-(2-Diisopropylamino)ethoxy-4-methoxyphenyl]-4-(4-methylphenyl)piperazine-1-carboxamide;

N-[3-(2-Diisopropylamino)ethoxy-4-methoxyphenyl]-4-(2,5-dimethylphenyl)piperazine-1-carboxamide;

N-[3-(2-Diisopropylamino)ethoxy-4-methoxyphenyl]-4-(3,4-dimethylphenyl)piperazine-1-carboxamide;

N-[3-(2-Diisopropylamino)ethoxy-4-methoxyphenyl]-4-(3,5-dichlorophenyl)piperazine-1-carboxamide;

N-[3-(2-Diisopropylamino)ethoxy-4-methoxyphenyl]-4-(3-methoxyphenyl)piperazine-1-carboxamide;

N-[3-(2-Diisopropylamino)ethoxy-4-methoxyphenyl]-4-(3,5-dimethoxyphenyl)piperazine-1-carboxamide;

N-[3-(2-Diisopropylamino)ethoxy-4-methoxyphenyl]-4-[3-(ethoxycarbonyl)phenyl]piperazine-1-carboxamide;

N-[3-(2-Diisopropylamino)ethoxy-4-methoxyphenyl]-4-(2-cyanophenyl)piperazine-1-carboxamide;

N-[3-(2-Diisopropylamino)ethoxy-4-methoxyphenyl]-4-(4-cyanophenyl)piperazine-1-carboxamide;

N-[3-(2-Diisopropylamino)ethoxy-4-methoxyphenyl]-4-(2-pyridinyl)piperazine-1-carboxamide;

N-[3-(2-Diisopropylamino)ethoxy-4-methoxyphenyl]-4-(4-pyridinyl)piperazine-1-carboxamide,

N-[3-(2-Diisopropylamino)ethoxy-4-methoxyphenyl]-4-[4-chloro-3-(trifluoromethyl)phenyl]piperazine-1-carboxamide;

N-[3-(2-Diisopropylamino)ethoxy-4-methoxyphenyl]-4-[2-methyl-3-(trifluoromethyl)phenyl]piperazine-1-carboxamide;

N-[3-(2-Diisopropylamino)ethoxy-4-methoxyphenyl]-4-(1-naphthalenyl)piperazine-1-carboxamide;

N-[3-(2-Diisopropylamino)ethoxy-4-methoxyphenyl]-4-[1-(5,6,7,8-tetrahydronaphthalenyl]piperazine-1-carboxamide;

N-[3-(2-Diisopropylamino)ethoxy-4-methoxyphenyl]-4-(1H-indol-4-yl)piperazine-1-carboxamide;

N-[3-(2-Diisopropylamino)ethoxy-4-methoxyphenyl]-4-(4-methoxyphenyl)-3-methylpiperazine-1-carboxamide;

N-[3-(2-Diisopropylamino)ethoxy-4-methoxyphenyl]-4-(5-chloro-2-methoxyphenyl)piperazine-1-carboxamide;

N-[3-(2-Diisopropylamino)ethoxy-4-methoxyphenyl]-4-(3-hydroxyphenyl)piperazine-1-carboxamide;

N-[3-(2-Diisopropylamino)ethoxy-4-methoxyphenyl]-4-(5-chloro-2-methylphenyl)piperazine-1-carboxamide;

N-[3-(2-Diisopropylamino)ethoxy-4-methoxyphenyl]-4-(3-chloro-2-methylphenyl)piperazine-1-carboxamide;

4-(2,3-Dimethylphenyl)-N-[4-methoxy-3-[1-(1-methylethyl)-4-piperidinyl]phenyl]-1-piperazinecarboxamide;

4-(2,3-Dichlorophenyl)-N-[4-methoxy-3-[1-(1-methylethyl)-4-piperidinyl]phenyl]-1-piperazinecarboxamide;

N-[3-(2-Diisopropylamino)ethoxy-4-methoxyphenyl]-4-(3-carboxyphenyl)piperazine-1-carboxamide;

N-[3-(2-Diisopropylamino)ethoxy-4-methoxyphenyl]-4-(2-carboxyphenyl)piperazine-1-carboxamide;

N-[3-(2-Diisopropylamino)ethoxy-4-methoxyphenyl]-4-(3,4-dimethoxyphenyl)-1-piperazinecarboxamide;

4-(2-Benzothiazolyl)-N-[3-(2-diisopropylamino)ethoxy-4-methoxyphenyl]-1-piperidinecarboxamide;

4-(2-Benzothiazolyl)-N-[4-methoxy-3-[1-(1-methylethyl)-4-piperidinyl]phenyl]-1-piperidinecarboxamide;

4-(1H-Indol-2-yl)-N-[3-(2-diisopropylamino)ethoxy-4-methoxyphenyl]-1-piperidinecarboxamide;

4-(1H-Indol-2-yl)-N-[4-methoxy-3-[1-(1-methylethyl)-4-piperidinyl]phenyl]-1-piperidinecarboxamide;

4-(4-Chlorophenyl)-N-[3-(2-diisopropylamino)ethoxy -4-methoxypheny]-4-hydroxy-1-piperidinecarboxamide;

4-(4-Chlorophenyl)-N-[4-methoxy-3-[1-(1-methylethyl)-4-piperidinyl]phenyl]-4-hydroxy-1-piperidinecarboxamide;

4-Acetyl-4-(4chlorophenyl)-N-[3-(2-diisopropylamino)ethoxy-4-methoxyphenyl]-1-piperidinecarboxamide;

4-Acetyl-4-(4-chlorophenyl)-N-[4-methoxy-3-[1-(1-methylethyl)-4-piperidinyl]phenyl]-1-piperidinecarboxamide;

4-(4-Chlorophenyl)-4-cyano-N-[3-(2-diisopropylamino)ethoxy-4-methoxyphenyl]-1-piperidinecarboxamide;

4-(4-Chlorophenyl)-4-cyano-N-[4-methoxy-3-[1-(1-methylethyl)-4-piperidinyl]phenyl]-1-piperidinecarboxamide;

N-[3-(2-Diisopropylamino)ethoxy-4-methoxyphenyl]-4-(4-hydroxyphenyl)-1-piperidinecarboxamide;

4-(4-Hydroxyphenyl)-N-[4-methoxy-3-[1-(1-methylethyl)-4-piperidinyl]phenyl]-1-piperidinecarboxamide;

4-(6-Chloro-2-benzothiazolyl)-N-[3-(2-diisopropylamino)ethoxy-4-methoxyphenyl]-1-piperazinecarboxamide;

4-(6-Chloro-2-benzothiazolyl)-N-[4-methoxy-3-[1-(1-methylethyl)-4-piperidinyl]phenyl]-1-piperazinecarboxamide;

N-[3-(2-Diisopropylamino)ethoxy-4-methoxyphenyl]-4-(2-pyrazinyl)-1-piperazinecarboxamide;

N-[4-Methoxy-3-[1-(1-methylethyl)-4-piperidinyl]phenyl]-4-(2-pyrazinyl)-1-piperazinecarboxamide;

N-[3-(2-Diisopropylamino)ethoxy-4-methoxyphenyl]-4-[5-(trifluoromethyl)-2-pyridinyl]-1-piperazinecarboxamide;

N-[4-Methoxy-3-[1-(1-methylethyl)-4-piperidinyl]phenyl]-4-[5-(trifluoromethyl)-2-pyridinyl]-1-piperazinecarboxamide;

4-(3,4-Dimethoxyphenyl)-N-[4-methoxy-3-[1-(1-methylethyl)-4-piperidinyl]phenyl]-1-piperazinecarboxamide;

4-(2-Chlorophenyl)-N-[4-methoxy-3-[1-(1-methylethyl)-4-piperidinyl]phenyl]-1-piperazinecarboxamide;

4-(3-Chlorophenyl)-N-[4-methoxy-3-[1-(1-methylethyl)-4-piperidinyl]phenyl]-1-piperazinecarboxamide;

4-(4-Chlorophenyl)-N-[4-methoxy-3-[1-(1-methylethyl)-4-piperidinyl]phenyl]-1-piperazinecarboxamide;

4-(3,4-Dichlorophenyl)-N-[4-methoxy-3-[1-(1-methylethyl)-4-piperidinyl]phenyl]-1-piperazinecarboxamide;

4-(3,5-Dichlorophenyl)-N-[4-methoxy-3-[1-(1-methylethyl)-4-piperidinyl]phenyl]-1-piperazinecarboxamide;

4-(2,6-Dimethylphenyl)-N-[4-methoxy-3-[1-(1-methylethyl)-4-piperidinyl]phenyl]-1-piperazinecarboxamide;

4-(2,4-Dimethylphenyl)-N-[4-methoxy-3-[1-(1-methylethyl)-4-piperidinyl]phenyl]-1-piperazinecarboxamide;

4-(3,5-Dimethylphenyl)-N-[4-methoxy-3-[1-(1-methylethyl)-4-piperidinyl]phenyl]-1-piperazinecarboxamide;

N-[4-Methoxy-3-[1-(1-methylethyl)-4-piperidinyl]phenyl]-4-(3-methylphenyl)-1-piperazinecarboxamide;

4-(2,5-Dimethylphenyl)-N-[4-methoxy-3-[1-(1-methylethyl)-4-piperidinyl]phenyl]-1-piperazinecarboxamide;

4-(3,4-Dimethylphenyl)-N-[4-methoxy-3-[1-(1-methylethyl)-4-piperidinyl]phenyl]-1-piperazinecarboxamide;

N-[4-Methoxy-3-[1-(1-methylethyl)-4-piperidinyl]phenyl]-4-(5,6,7,8-tetrahydro-1-naphthalenyl)-1-piperazinecarboxamide;

N-[4-Methoxy-3-[1-(1-methylethyl)-4-piperidinyl]phenyl]-4-(2-methylphenyl)-1-piperazinecarboxamide;

4-(5-Chloro-2-methylphenyl)-N-[4-methoxy-3-[1-(1-methylethyl)-4-piperidinyl]phenyl]-1-piperazinecarboxamide;

4-(3-Chloro-2-methylphenyl)-N-[4-methoxy-3-[1-(1-methylethyl)-4-piperidinyl]phenyl]-1-piperazinecarboxamide;

4-(3-Chloro-2-methoxyphenyl)-N-[4-methoxy-3-[1-(1-methylethyl)-4-piperidinyl]phenyl]-1-piperazinecarboxamide;

N-[4-Methoxy-3-[1-(1-methylethyl)-4-piperidinyl]phenyl]-4-[3-(trifluoromethyl)phenyl]-1-piperazinecarboxamide;

4-[4-Chloro-3-(trifluoromethyl)phenyl]-N-[4-methoxy-3-[1-(1-methylethyl)-4-piperidinyl]phenyl]-1-piperazinecarboxamide;

N-[4-Methoxy-3-[1-(1-methylethyl)-4-piperidinyl]phenyl]-4-[2-methyl-3-(trifluoromethyl)phenyl]-1-piperazinecarboxamide;

4-(3-Methoxyphenyl)-N-[4-methoxy-3-[1-(1-methylethyl)-4-piperidinyl]phenyl]-1-piperazinecarboxamide;

4-(3,5-Dimethoxyphenyl)-N-[4-methoxy-3-[1-(1-methylethyl)-4-piperidinyl]phenyl]-1-piperazinecarboxamide;

4-(2-Cyanophenyl)-N-[4-methoxy-3-[1-(1-methylethyl)-4-piperidinyl]phenyl]-1-piperazinecarboxamide;

4-(4-Cyanophenyl)-N-[4-methoxy-3-[1-(1-methylethyl)-4-piperidinyl]phenyl]-1-piperazinecarboxamide;

4-[3-(Ethoxycarbonyl)phenyl]-N-[4-methoxy-3-[1-(1-methylethyl)-4-piperidinyl]phenyl]-1-piperazinecarboxamide;

4-(1H-Indol-4-yl)-N-[4-methoxy-3-[1-(1-methylethyl)-4-piperidinyl]phenyl]-1-piperazinecarboxamide;

4-[3-(Ethoxycarbonyl)phenyl]-N-[3-(3-diisopropylamino)propoxy-4-methoxyphenyl]-1-piperazinecarboxamide; and

N-[3-(2-Diisopropylamino)ethoxy-4-methoxyphenyl]-4-(4-carboxyphenyl)piperazine-1-carboxamide.

Among the more preferred compounds of the invention are the following compounds:

N-[3-(2-Diisopropylamino)ethoxy-4-methoxyphenyl]-4-(2,3-dimethylphenyl)piperazine-1-carboxamide;

N-[3-(2-Diisopropylamino)ethoxy-4-methoxyphenyl]-4-(2,3-dichlorophenyl)piperazine-1-carboxamide;

N-[3-(2-Diisopropylamino)ethoxy-4-methoxyphenyl]-4-(2,4-dimethylphenyl)piperazine-1-carboxamide;

N-[2,3-Dihydro-1′-isopropyl-spiro[benzofuran-5-yl-3,4′-piperidine]]-4-(2,3-dimethylphenyl)piperazine-1-carboxamide,

N-[3-(2-Diisopropylamino)ethoxy-4-methoxyphenyl]-4-[3-(ethoxycarbonyl)phenyl]piperazine-1-carboxamide;

N-[3-(2-Diisopropylamino)ethoxy-4-methoxyphenyl]-4-[2-methyl-3-(trifluoromethyl)phenyl]piperazine-1-carboxamide;

N-[3-(2-Diisopropylamino)ethoxy-4-methoxyphenyl]-4-[1-(5,6,7,8-tetrahydronaphthalenyl]piperazine-1-carboxamide;

N-[3-(2-Diisopropylamino)ethoxy-4-methoxyphenyl]-4-(5-chloro-2-methoxyphenyl)piperazine-1-carboxamide;

4-(6-Chloro-2-benzothiazolyl)-N-[4-methoxy-3-[1-(1-methylethyl)-4-piperidinyl]phenyl]-1-piperazinecarboxamide;

4-(2-Chlorophenyl)-N-[4-methoxy-3-[1-(1-methylethyl)-4-piperidinyl]phenyl]-1-piperazinecarboxamide;

4-(3-Chlorophenyl)-N-[4-methoxy-3-[1-(1-methylethyl)-4-piperidinyl]phenyl]-1-piperazinecarboxamide;

4-(4-Chlorophenyl)-N-[4-methoxy-3-[1-(1-methylethyl)-4-piperidinyl]phenyl]-1-piperazinecarboxamide;

4-(3,4-Dichlorophenyl)-N-[4-methoxy-3-[1-(1-methylethyl)-4-piperidinyl]phenyl]-1-piperazinecarboxamide;

4-(3,5-Dichlorophenyl)-N-[4-methoxy-3-[1-(1-methylethyl)-4-piperidinyl]phenyl]-1-piperazinecarboxamide;

4-(2,4-Dimethylphenyl)-N-[4-methoxy-3-[1-(1-methylethyl)-4-piperidinyl]phenyl]-1-piperazinecarboxamide;

4-(3,5-Dimethylphenyl)-N-[4-methoxy-3-[1-(1-methylethyl)-4-piperidinyl]phenyl]-1-piperazinecarboxamide;

N-[4-Methoxy-3-[1-(1-methylethyl)-4-piperidinyl]phenyl]-4-(3-methylphenyl)-1-piperazinecarboxamide;

4-(2,5-Dimethylphenyl)-N-[4-methoxy-3-[1-(1-methylethyl)-4-piperidinyl]phenyl]-1-piperazinecarboxamide;

4-(3,4-Dimethylphenyl)-N-[4-methoxy-3-[1-(1-methylethyl)-4-piperidinyl]phenyl]-1-piperazinecarboxamide;

N-[4-Methoxy-3-[1-( 1-methylethyl)-4-piperidinyl]phenyl]-4-(5,6,7,8-tetrahydro-1-naphthalenyl)-1-piperazinecarboxamide;

N-[4-Methoxy-3-[1-(1-methylethyl)-4-piperidinyl]phenyl]-4-(2-methylphenyl)-1-piperazinecarboxamide;

4-(5-Chloro-2-methylphenyl)-N-[4-methoxy-3-[1-(1-methylethyl)-4-piperidinyl]phenyl]-1-piperazinecarboxamide;

4-(3-Chloro-2-methylphenyl)-N-[4-methoxy-3-[1-(1-methylethyl)-4-piperidinyl]phenyl]-1-piperazinecarboxamide;

4-(3-Chloro-2-methoxyphenyl)-N-[4-methoxy-3-[1-(1-methylethyl)-4-piperidinyl]phenyl]-1-piperazinecarboxamide;

N-[4-Methoxy-3-[1-(1-methylethyl)-4-piperidinyl]phenyl]-4-[3-(trifluoromethyl)phenyl]1-piperazinecarboxamide;

4-[4-Chloro-3-(trifluoromethyl)phenyl]-N-[4-methoxy-3-[1-(1-methylethyl)-4-piperidinyl]phenyl]-1-piperazinecarboxamide;

N-[4-Methoxy-3-[1-(1-methylethyl)-4-piperidinyl]phenyl]-4-[2-methyl-3-(trifluoromethyl)phenyl]-1-piperazinecarboxamide;

4-(3-Methoxyphenyl)-N-[4-methoxy-3-[1-(1-methylethyl)-4-piperidinyl]phenyl]-1-piperazinecarboxamide;

4-(3,5-Dimethoxyphenyl)-N-[4-methoxy-3-[1-(1-methylethyl)-4-piperidinyl]phenyl]-1-piperazinecarboxamide;

4-(2-Cyanophenyl)-N-[4-methoxy-3-[1-(1-methylethyl)-4-piperidinyl]phenyl]-1-piperazinecarboxamide;

4-[3-(Ethoxycarbonyl)phenyl]-N-[4-methoxy-3-[1-(1-methylethyl)-4-piperidinyl]phenyl]-1-piperazinecarboxamide;

4-(1H-Indol-4-yl)-N-[4-methoxy-3-[1-(1-methylethyl)-4-piperidinyl]phenyl]-1-piperazinecarboxamide;

N-[4-Methoxy-3-[4-cyano-1-(1-methylethyl)-4-piperidinyl]phenyl]-4-(5,6,7,8-tetrahydro-1-naphthalenyl)-1-piperazinecarboxamide;

4-[3-(Ethoxycarbonyl)phenyl]-N-[3-(3-diisopropylamino)propoxy-4-methoxyphenyl]-1-piperazinecarboxamide; and

N-[3-(2-Diisopropylamino)ethoxy-4-methoxyphenyl]-4-(3-chloro-2-methylphenyl)piperazine-1-carboxamide.

Among the most preferred compounds of the invention are the following compounds:

4-(3,5-Dichlorophenyl)-N-[4-methoxy-3-[1-(1-methylethyl)-4-piperidinyl]phenyl]-1-piperazinecarboxamide;

N-[4-Methoxy-3-[1-(1-methylethyl)-4-piperidinyl]phenyl]-4-(5,6,7,8-tetrahydro-1-naphthalenyl)-1-piperazinecarboxamide;

4-(3-Chloro-2-methylphenyl)-N-[4-methoxy-3-[1-(1-methylethyl)-4-piperidinyl]phenyl]-1-piperazinecarboxamide;

N-[4-Methoxy-3-[1-(1-methylethyl)-4-piperidinyl]phenyl]-4-[2-methyl-3-(trifluoromethyl)phenyl]-1-piperazinecarboxamide;

4-(1H-Indol-4-yl)-N-[4-methoxy-3-[1-(1-methylethyl)-4-piperidinyl]phenyl]-1-piperazinecarboxamide; and

N-[3-(2-Diisopropylamino)ethoxy-4-methoxyphenyl]-4-[1-(5,6,7,8-tetrahydronaphthalenyl]piperazine-1-carboxamide.

Formulation of Pharmaceutical Compositions

The pharmaceutically effective compounds of this invention (and the pharmaceutically acceptable salts thereof) are administered in conventional dosage forms prepared by combining a compound of formula (I) (“active ingredient”) in an amount sufficient to treat COPD, asthma and atopic disorders (for example, atopic dermatitis and allergies), rheumatoid arthritis, sarcoidosis, or idiopathic pulmonary fibrosis and other fibrotic diseases, atherosclerosis, psoriasis, autoimmune diseases such as multiple sclerosis, treating and/or preventing rejection of transplanted organs, inflammatory bowel disease, and HIV infection, (“CCR5-mediated disease states”) with standard pharmaceutical carriers or diluents according to conventional procedures well known in the art. These procedures may involve mixing, granulating and compressing or dissolving the ingredients as appropriate to the desired preparation.

The pharmaceutical carrier employed may be, for example, either a solid or liquid. Exemplary of solid carriers are lactose, terra alba, sucrose, talc, gelatin, agar, pectin, acacia, magnesium stearate, stearic acid and the like. Exemplary of liquid carriers are syrup, peanut oil, olive oil, water and the like. Similarly, the carrier or diluent may include time delay material well known to the art, such as glyceryl monostearate or glyceryl distearate alone or with a wax.

A wide variety of pharmaceutical forms can be employed. Thus, if a solid carrier is used, the preparation can be tableted, placed in a hard gelatin capsule in powder or pellet form or in the form of a troche or lozenge. The amount of solid carrier will vary widely but preferably will be from about 25 mg to about 1000 mg. When a liquid carrier is used, the preparation will be in the form of a syrup, emulsion, soft gelatin capsule, sterile injectable liquid such as an ampule or nonaqueous liquid suspension.

The active ingredient may also be administered topically to a mammal in need of treatment or prophylaxis of CCR5 mediated disease states. The amount of active ingredient required for therapeutic effect on topical administration will, of course, vary with the compound chosen, the nature and severity of the disease state being treated and the mammal undergoing treatment, and is ultimately at the discretion of the physician. A suitable dose of an active ingredient is 1.5 mg to 500 mg for topical administration, the most preferred dosage being 1 mg to 100 mg, for example 5 to 25 mg administered two or three times daily.

By topical administration is meant non-systemic administration and includes the application of the active ingredient externally to the epidermis, to the buccal cavity and instillation of such a compound into the ear, eye and nose, and where the compound does not significantly enter the blood stream. By systemic administration is meant oral, intravenous, intraperitoneal and intramuscular administration.

While it is possible for an active ingredient to be administered alone as the raw chemical, it is preferable to present it as a pharmaceutical formulation. The active ingredient may comprise, for topical administration, from 0.001% to 10% w/w, e.g. from 1% to 2% by weight of the formulation although it may comprise as much as 10% w/w but preferably not in excess of 5% w/w and more preferably from 0.1% to 1% w/w of the formulation.

The topical formulations of the present invention, both for veterinary and for human medical use, comprise an active ingredient together with one or more acceptable carrier(s) therefor and optionally any other therapeutic ingredient(s). The carrier(s) must be ‘acceptable’ in the sense of being compatible with the other ingredients of the formulation and not deleterious to the recipient thereof.

Formulations suitable for topical administration include liquid or semi-liquid preparations suitable for penetration through the skin to the site of inflammation such as liniments, lotions, creams, ointments or pastes, and drops suitable for administration to the eye, ear or nose.

Drops according to the present invention may comprise sterile aqueous or oily solutions or suspensions and may be prepared by dissolving the active ingredient in a suitable aqueous or alcoholic solution of a bactericidal and/or fungicidal agent and/or any other suitable preservative, and preferably including a surface active agent. The resulting solution may then be clarified by filtration, transferred to a suitable container which is then sealed and sterilized by autoclaving or maintaining at 98-100° C. for half an hour. Alternatively, the solution may be sterilized by filtration and transferred to the container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Lotions according to the present invention include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil.

Creams, ointments or pastes according to the present invention are semi-solid formulations of the active ingredient for external application. They may be made by mixing the active ingredient in finely-divided or powdered form, alone or in solution or suspension in an aqueous or non-aqueous fluid, with the aid of suitable machinery, with a greasy or non-greasy basis. The basis may comprise hydrocarbons such as hard, soft or liquid paraffin, glycerol, beeswax, a metallic soap; a mucilage; an oil of natural origin such as almond, corn, arachis, castor or olive oil; wool fat or its derivatives, or a fatty acid such as stearic or oleic acid together with an alcohol such as propylene glycol. The formulation may incorporate any suitable surface active agent such as an anionic, cationic or non-ionic surfactant such as esters or polyoxyethylene derivatives thereof. Suspending agents such as natural gums, cellulose derivatives or inorganic materials such as silicaceous silicas, and other ingredients such as lanolin, may also be included.

The active ingredient may also be administered by inhalation. By “inhalation” is meant intranasal and oral inhalation administration. Appropriate dosage forms for such administration, such as an aerosol formulation or a metered dose inhaler, may be prepared by conventional techniques. The daily dosage amount of the active ingredient administered by inhalation is from about 0.1 mg to about 100 mg per day, preferably about 1 mg to about 10 mg per day.

In one aspect, this invention relates to a method of treating COPD, asthma and atopic disorders (for example, atopic dermatitis and allergies), rheumatoid arthritis, sarcoidosis, or idiopathic pulmonary fibrosis and other fibrotic diseases, atherosclerosis, psoriasis, autoimmune diseases such as multiple sclerosis, treating and/or preventing rejection of transplanted organs, inflammatory bowel disease, and HIV infection, all in mammals, preferably humans, which comprises administering to such mammal an effective amount of a CCR5 receptor modulator, in particular, a compound as depicted in formula (I).

By the term “treating” is meant either prophylactic or therapeutic therapy. Such formula (I) compound can be administered to such mammal in a conventional dosage form prepared by combining the formula (I) compound with a conventional pharmaceutically acceptable carrier or diluent according to known techniques. It will be recognized by one of skill in the art that the form and character of the pharmaceutically acceptable carrier or diluent is dictated by the amount of active ingredient with which it is to be combined, the route of administration and other well-known variables. The formula (I) compound is administered to a mammal in need of treatment for COPD, asthma and atopic disorders (for example, atopic dermatitis and allergies), rheumatoid arthritis, sarcoidosis, or idiopathic pulmonary fibrosis and other fibrotic diseases, atherosclerosis, psoriasis, autoimmune diseases such as multiple sclerosis, treating and/or preventing rejection of transplanted organs, inflammatory bowel disease, and HIV infection, in an amount sufficient to decrease symptoms associated with these disease states. The route of administration may be oral or parenteral.

In another aspect, the invention relates to a method for modulating factors which exacerbate the symptoms of the CCR5-mediated diseases described herein.

The term parenteral as used herein includes intravenous, intramuscular, subcutaneous, intra-rectal, intravaginal or intraperitoneal administration. The subcutaneous and intramuscular forms of parenteral administration are generally preferred. The daily parenteral dosage regimen will preferably be from about 30 mg to about 300 mg per day of active ingredient. The daily oral dosage regimen will preferably be from about 100 mg to about 2000 mg per day of active ingredient.

It will be recognized by one of skill in the art that the optimal quantity and spacing of individual dosages of a formula (I) compound will be determined by the nature and extent of the condition being treated, the form, route and site of administration, and the particular mammal being treated, and that such optimums can be determined by conventional techniques. It will also be appreciated by one of skill in the art that the optimal course of treatment, i.e., the number of doses of the formula (I) compound given per day for a defined number of days, can be ascertained by those skilled in the art using conventional course of treatment determination tests.

Methods of Preparation

The compounds of formula (I) can be prepared by art-recognized procedures from known or commercially available starting materials. If the starting materials are unavailable from a commercial source, their synthesis is described herein, or they can be prepared by procedures known in the art.

For example, as shown in Scheme 1, compounds of formula (I) where L′ is NR^{30′} are prepared by treating a suitably substituted aniline 1-1 with suitable reagent, for example triphosgene, and a suitable base, for example triethylamine, in a suitable solvent, for example dichloromethane, followed by treatment with a suitably substituted amine 1-2, e.g., 1-(5,6,7,8-tetrahydro-1-naphthalenyl)piperazine, ethyl 3-(1-piperazinyl)benzoate, 4-(phenyl)piperidine, 1-(phenyl)piperazine, 4-phenyl-2,3,4,6-tetrahydropyridine, hexahydro-1-phenyl-1H-1,4-diazepine, etc., to afford the title compound 1-3.

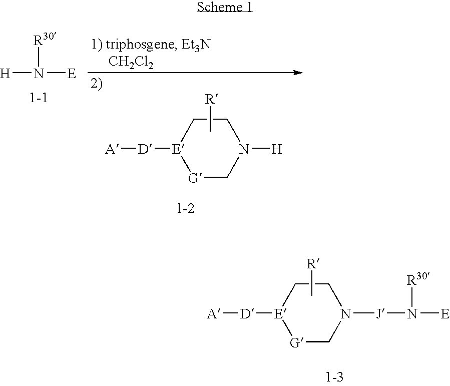

Suitably substituted anilines used to prepare compounds of formula (I) where E is a group of formula (a) are prepared according to the methods of international application publication number WO 95/15954, published Jun. 15, 1995, international application publication number WO 95/17398, published Jun. 29, 1995, international application publication number WO 95/26328, published Oct. 5, 1995, and international application publication number WO 96/06079, published Feb. 29, 1996.

Suitably substituted anilines used to prepare compounds of formula (I) where E is a group of formula (b) are prepared according to the methods of international application publication number WO 95/11934, published Apr. 25, 1995, and WO 95/19477, published Jun. 27, 1995. Four other applications relate to the spiro compounds WO 97/17350 published May 15, 1997; WO 97/34900 published Sep. 25, 1997; WO 97/34901 published Sep. 25, 1997; WO 97/35862 published Oct. 2, 1997.

Suitably substituted anilines used to prepare compounds of formula (I) where E is a group of formula (c) are prepared according to the methods of international application publication number WO 95/30675, published Nov. 16, 1995.

Suitably substituted anilines used to prepare compounds of formula (I) where E is a group of formula (f) are prepared according to the methods of international application publication number WO 95/17401, published Jun. 29, 1995.

Suitably substituted anilines used to prepare compounds of formula (I) where E is a group or formula (g) are prepared according to the methods of international application publication number WO 96/31508 published Oct. 10, 1996.

Suitably substituted anilines used to prepare compounds of formula (I) where E is a group of formula (h) are prepared according to the methods of international application publication number WO 95/32967, published Dec. 7, 1995 and WO 97/07120, published Feb. 27, 1997, WO 97/07120, published Feb. 27, 1997.

Suitably substituted anilines used to prepare compounds of formula (I) where E is a group of formula (i) are prepared according to the methods of international application publication number WO 97/19070 published May 29, 1997.

Novel intermediates useful in preparing compounds of formula (I) are also included in the scope of this invention. For example, as shown in Scheme 2, certain anilines wherein E is a group (g) are prepared from commercially available 4-(2-methoxyphenyl)piperidine, 2-1 by treatment with a suitable acylating agent, for example trifluoroacetic anhydride, and suitable base, for example triethylamine, in a suitable solvent, for example dichloromethane. Nitration of the resulting N-acylated phenylpiperidine with a suitable nitrating agent, for example 70% nitric acid in acetic anhydride, at a suitable temperature, for example 0° C., for a suitable time, for example 30 minutes, yields 2-2. Removal of the piperidine nitrogen protecting group from 2-2 with a suitable reagent, for example potassium carbonate, in a suitable solvent, for example aqueous methanol, at a suitable temperature, for example room temperature, gives 2-3 where R is H. Treatment of 2-3 where R is H with a suitable alkylating agent RX where R is C₁₋₆alkyl or C₃₋₇cycloalkyl, for example isopropyl, and X is a suitable leaving group, for example iodo, bromo, methanesulfonyloxy, trifluoromethysulfonyloxy, etc., and with a suitable base, for example potassium carbonate, in a suitable solvent, for example dimethylformamide and acetonitrile, at a suitable temperature, for example 70° C., for a suitable time, for example 20 hours gives 2-3 where R is C₁₋₆alkyl or C₃₋₇cycloalkyl. Alternatively, 2-3 where R is H may be reductively alkylated on the piperidine nitrogen by treatment with a C₁₋₆aldehyde, C₃₋₆ketone, or a C₃₋₇cyclic ketone, for example, cyclopentanone, and a suitable reducing agent, for example sodium cyanoborohydride, in a suitable solvent, for example, acetic acid and methanol, for a suitable time, for example 16 hours, to afford 2-3 where R is C₁₋₆alkyl or C₃₋₇cycloalkyl. Reduction of the nitro group in 2-3 where R is C₁₋₆alkyl or C₃₋₇cycloalkyl with a suitable reagent, for example hydrogen, in the presence of a suitable catalyst, for example palladium hydroxide, in a suitable solvent, for example ethanol, for a suitable time, for example 4 hours, affords 2-4. Compounds 2-4 are examples of 1-1 in Scheme 1 and are converted to 1-3, which are compounds of formula (I)

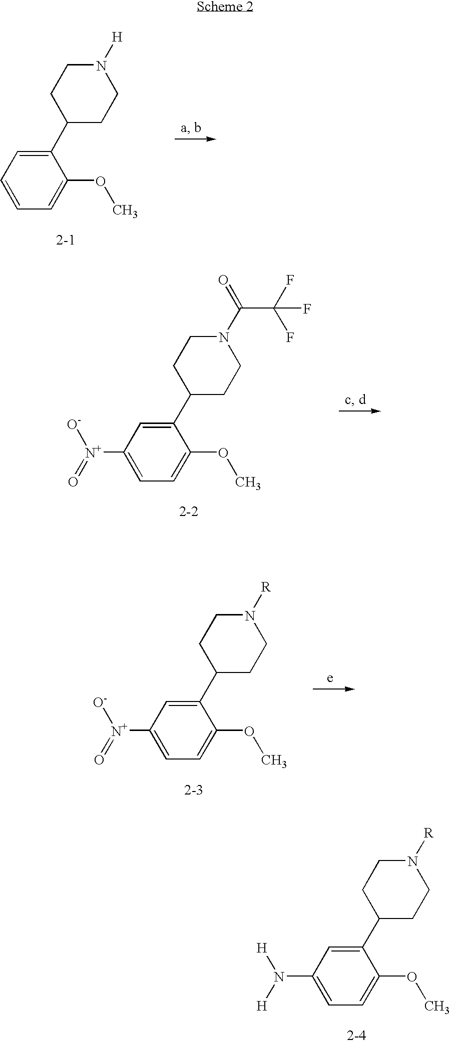

(a) TFAA, Et₃N, CH₂Cl₂, 16 h; (b) HNO₃, Ac₂O, 0° C., 30 min; (c) K₂CO₃, MeOH, H₂O, 40 h; (d) K₂CO₃, RX, DMF, MeCN, 70° C., 20 h or RCHO/RRCO, NaBH₃CN, AcOH, MeOH, Δ, 16 h; (e) H₂, Pd(OH)₂, EtOH, 4 h.

Particularly useful intermediates for preparing compounds of formula (I) are:

4-methoxy-3-[1-(1-methylethyl)-4-piperidinyl]benzenamine;

4-methoxy-3-[1-cyclopentyl-4-piperidinyl]benzenamine; and

4-methoxy-3-[1-(3-pentyl)-4-piperidinyl]benzenamine.

The invention will now be described by reference to the following examples which are merely illustrative and are not to be construed as a limitation of the scope of the present invention. In the Examples, mass spectra were performed upon a VG Zab mass spectrometer using fast atom bombardment, unless otherwise indicated.

### EXAMPLES

### Preparation 1

### Preparation of 4-Methoxy-3-[1-(1-methylethyl)-4-piperidinyl)benzenamine

a) 4-(2-methoxyphenyl)-1-(trifluoroacetyl)piperidine

Trifluoroacetic anhydride (8.1 g, 39 mmol) was added portionwise over 10 min to a solution of commercially available 4-(2-methoxyphenyl)piperidine (6.7 g, 35 mmol), triethylamine (7.8 g, 77 mmol), and dichloromethane (100 mL) at RT. The reaction was maintained at RT for 16 h. The resultant mixture was washed with saturated sodium bicarbonate, saturated ammonium chloride, and with brine, dried (MgSO₄), and concentrated in vacuo to afford 10 g (99%) of the title compound as an amber oil. MS(ES) m/e 288.1 [M+H]⁺.

b) 4-(2-methoxy-5-nitrophenyl)-1-(trifluoroacetyl)piperidine

Nitric acid (70%, 3.1 mL) was added portionwise to a solution of the compound of Preparation 1(a) (5.0 g, 17 mmol) in acetic anhydride (17 mL) at 0° C. The mixture was maintained at 0° C. for an additional 30 min, combined with an identical concurrently run reaction, and poured into water (600 mL). The pH of the resultant mixture was adjusted to >9 by the addition of aqueous sodium carbonate followed by 10% sodium hydroxide. The resulting mixture was extracted with dichloromethane (2×400 mL) and the combined organic layers were washed with brine, dried (MgSO₄), and concentrated in vacuo to give 12 g (>100%) of a 2.2:1 mixture of the title compound and its 3-nitro isomer. The crude product was recrystallized from methanol (30 mL) to give 5.9 g (54%) of the title compound as off-white crystals. MS(ES) m/e 333.1 [M+H]⁺.

c) 4-(2-methoxy-5-nitrophenyl)piperidine

Potassium carbonate (10 g, 74 mmol) was added to a solution of the compound of Preparation 1 (b) (4.9 g, 15 mmol), methanol (100 mL) and water (7.5 mL). The resultant mixture was stirred at RT for 40 h, concentrated in vacuo, and the residue partitioned between water and dichloromethane. The layers were separated and aqueous layer was extracted with dichloromethane. The combined organic layers were washed with brine, dried (MgSO₄), and concentrated in vacuo to give 3.7 g (>100%) of the title compound as an off-white solid. MS(ES) m/e 237.2 [M+H]⁺.

d) 4-(2-methoxy-5-nitrophenyl)-1-(1-methylethyl)piperidine

Potassium carbonate (8.6 g, 62 mmol) and isopropyl iodide (8.0 g, 47 mmol) were added to a solution of the compound of Preparation 1(c) (3.7 g, 16 mmol), dimethylformamide (10 mL) and acetonitrile (50 mL). The resultant mixture was heated at 70° C. for 20 h, concentrated in vacuo, and the residue partitioned between water and dichloromethane. The aqueous phase was extracted with dichloromethane and the combined organic layers were washed with water (3×100 mL) and with brine, dried (MgSO₄), and concentrated in vacuo to provide 4.0 g (90%) of the title compound as a yellow solid. MS(ES) m/e 279.2 [M+H]⁺.

e) 4-methoxy-3-[1-(1-methylethyl)4-piperidinyl)benzenamine

Palladium hydroxide on carbon (1.2 g, 20% dry weight) was added to a solution of the compound of Preparation 1(d) (4.0 g, 14 mmol) in ethanol (100 mL). The mixture was hydrogenated at 50 psi for 4 h, filtered through Celite®, and concentrated in vacuo. The residue was dissolved in ether (200 mL) and washed with 10% sodium carbonate and with water (2×100 mL). The ether solution was dried (MgSO₄) and concentrated in vacuo to provide 3.0 g (84%) of the title compound as a tan solid. MS(ES) m/e 249.2 [M+H]⁺.

### Preparation 2

### Preparation of 1-(5,6,7,8-Tetrahydro-1-naphthalenyl)piperazine

Following the general procedure of Kuipers, et. al., J. Med. Chem., 1995, 38, 1942-1954, bis(chloroethyl)amine hydrochloride (2 g, 11.2 mmol) was added to a solution of 5,6,7,8-tetrahydro-1-naphthylamine (1.65 g, 11.2 mmol) in chlorobenzene (15 mL) and the mixture was heated to 135° C. for 2 days. The mixture was cooled, concentrated in vacuo, and the residue was purified by flash chromatography (silica gel, 5% methanol/dichloromethane) to give the title compound as a tan solid which was further purified by HPLC (YMC CombiPrep ODS-A, 50×20 mm, 20 mL/min, A:0.1% trifluoroacetic acid in acetonitrile B:0.1% aqueous trifluoroacetic acid, A:10 to 90% during 10 min, UV detection at 254 nm) to give the title compound as a tan solid (0.25 g).

### Preparation 3

### Preparation of Ethyl 3-(1-piperazinyl)benzoate

Following the general procedure of Kato et. al., WO 9802432 and of Preparation 2, except substituting ethyl 3-aminobenzoate for 5,6,7,8-tetrahydro-1-naphthylamine, gave the title compound. MS(ES) m/e 235.2 [M+H]⁺.

### Preparation 4

### Preparation of 4-Methoxy-3-[1-cyclopentyl-4-piperidinyl]benzenamine

a) 4-(2-methoxy-5-nitrophenyl)-1-(cyclopentyl)piperidine

A solution of the compound of Preparation 1(c) (3.4 g, 14.4 mmol) in methanol (21 mL) was treated with acetic acid (8.5 g, 0.14 mmol), cyclopentanone (6.12 g, 71.4 mmol) and sodium cyanoborohydride (3.74 g, 57.8 mmol). The resulting mixture was heated to reflux for 16 h, concentrated in vacuo, and the residue was partitioned between dichloromethane and 2N sodium hydroxide. The organic phase was dried (MgSO₄) and concentrated in vacuo to afford the title compound.

b) 4-methoxy-3-[1-cyclopentyl-4-piperidinyl]benzenamine

Following the general procedure of Preparation 1(e), except substituting the compound of Preparation 4(a) for the compound of Preparation 1(d), gave the title compound.

### Preparation 5

### Preparation of 4-Methoxy-3-[1-(3-pentyl)-4-piperidinyl]benzenamine

The title compound is prepared following the procedure of Preparation 4(a)-4(b), except substituting 3-pentanone for cyclopentanone.

### Example 1

### Preparation of N-[3-(2-Diisopropylamino)ethoxy-4-methoxyphenyl]-4-phenyl-1,2,3,6-tetrahydropyridine-1-carboxamide

A solution of triphosgene (0.23 g, 0.77 mmol) in dichloromethane (25 mL) was stirred in an ice bath and treated with a solution of 4-phenyl-1,2,3,6-tetrahydropyridine hydrochloride (0.5 g, 2.6 mmol) and triethylamine (1 g, 10.2 mmol) in dichloromethane added dropwise. The ice bath was removed and the mixture was stirred for 30 min, treated with 3-(2-diisopropylamino)ethoxy-4-methoxyaniline (WO 95/15954) (0.68 g, 2.55 mmol), and stirred for 16 h. The mixture was diluted with dichloromethane (50 mL), extracted with 5% sodium carbonate, dried (Na₂SO₄), and concentrated in vacuo. The residue was chromatographed (silica gel, 8% methanol/dichloromethane saturated with ammonia) to give the title compound. MS(ES) m/e 452.0 [M+H]⁺.

### Example 2

### Preparation of N-[3-(2-Diisopropylamino)ethoxy-4-methoxyphenyl]-4-(2,3-dimethylphenyl) piperazine-1-carboxamide

Triphosgene (74 mg, 0.25 mmol) was added to a solution of 3-(2-diisopropylamino)ethoxy-4-methoxyaniline (WO 95/15954) (200 mg, 0.75 mmol) and dichloromethane (3 mL) and maintained at RT for 30 min. Triethylamine (0.30 g, 0.42 mL, 3.0 mmol) was added and the resulting mixture was stirred for 1 h, treated with 1-(2,3-dimethylphenyl)piperazine (0.11 g, 0.60 mmol), and the mixture stirred at RT for 16 h. The mixture was washed with water, dried (MgSO₄) and concentrated in vacuo. The crude product was purified by chromatography (silica gel, 20:1:0.04 dichloromethane:methanol:triethylamine) to give 205 mg (70%) of the title compound as an off-white powder. MS(BS) m/e 483.1 [M+H]⁺.

### Examples 3-22

Following the procedure of Example 2, except substituting 1-phenylpiperazine, 1-(2-methylphenyl)piperazine, 1-[2-(acetamidomethyl)phenyl]-piperazine(GB 2309458), 1-[3-(trifluoromethyl)phenyl]piperazine, 1-(2-methoxyphenyl)piperazine, 1-(2-chlorophenyl)piperazine, 1-(3-chlorophenyl)piperazine, 1-(4-chlorophenyl)piperazine, 1-(2,6-dimethylphenyl)piperazine, 1-(2,3-dichlorophenyl)piperazine, and 1-(3,4-dichlorophenyl)piperazine for 1-(2,3-dimethylphenyl)piperazine, gave the following compounds:

N-[3-(2-diisopropylamino)ethoxy-4-methoxyphenyl]-4-phenylpiperazine-1-carboxamide: MS(ES) m/e 454.9 [M+H]⁺;

N-[3-(2-diisopropylamino)ethoxy-4-methoxyphenyl]-4-(2-methylphenyl)piperazine-1-carboxamide: MS(ES) m/e 469.1 [M+H]⁺;

N-[3-(2-diisopropylamino)ethoxy-4-methoxyphenyl]-4-(2-acetamido-methylphenyl)piperazine-1-carboxamide: MS(ES) m/e 525.9 [M+H]⁺;

N-[3-(2-diisopropylamino)ethoxy-4-methoxyphenyl]-4-(3-trifluoromethylphenyl)piperazine-1-carboxamide: MS(ES) m/e 522.8 [+H]⁺;

N-[3-(2-diisopropylamino)ethoxy-4-methoxyphenyl]-4-(2-methoxyphenyl)piperazine-1-carboxamide: MS(ES) m/e 485.0 [M+H]⁺;

N-[3-(2-diisopropylamino)ethoxy-4-methoxyphenyl]-4(2-chlorophenyl)piperazine-1-carboxamide: MS(ES) m/e 488.9 [M+HM]⁺;

N-[3-(2-diisopropylamino)ethoxy-4-methoxyphenyl]-4-(3-chlorophenyl)piperazine-1-carboxamide: MS(ES) m/e 488.8 [M+M]⁺;

N-[3-(2-diisopropylamino)ethoxy-4-methoxyphenyl]-4-(4-chlorophenyl)piperazine-1-carboxamide: MS(ES) m/e 488.8 [M+H]⁺;

N-[3-(2-diisopropylamino)ethoxy-4-methoxyphenyl]-4-(2,6-dimethylphenyl)piperazine-1-carboxamide: MS(ES) m/e 483.1 [M+H]⁺;

N-[3-(2-diisopropylamino)ethoxy-4-methoxyphenyl]-4-(2,3-dichlorophenyl)piperazine-1-carboxamide: MS(ES) m/e 522.9 [M+H]⁺;

N-[3-(2-diisopropylamino)ethoxy-4-methoxyphenyl]-4-(3,4dichlorophenyl)piperazine-1-carboxamide: MS(ES) m/e 522.7 [M+H]⁺;

N-[3-(2-Diisopropylamino)ethoxy-4-methoxyphenyl]-3-methyl-4-(3-methylphenyl)piperazine-1-carboxamide: MS(ES) m/e 483.2 [M+H]⁺;

N-[3-(2-Diisopropylamino)ethoxy-4-methoxyphenyl]-4-(4-methoxyphenyl)piperazine-1-carboxamide: MS(ES) m/e 499.2 [M+H]⁺;

N-[3-(2-Diisopropylamino)ethoxy-4-methoxyphenyl]-4-(2,4-dimethylphenyl)piperazine-1-carboxamide: MS(ES) m/e 483.2 [M+H]⁺;

N-[3-(2-Diisopropylamino)ethoxy-4-methoxyphenyl]-3-methyl-4-phenylpiperazine-1-carboxamide: MS(ES) m/e 469.2 [M+H]⁺;

N-[3-(2-Diisopropylamino)ethoxy-4-methoxyphenyl]-4-(3,4-dihydro-2(1H)-quinolinone-6-yl)piperazine-1-carboxamide: MS(ES) m/e 524.2 [M+H]⁺;

-[3-(2-Diisopropylamino)ethoxy-4-methoxyphenyl]-4-(3,5-dimethylphenyl)piperazine-1-carboxamide: MS(ES) m/e 483.2 [M+H]⁺;

N-[3-(2-Diisopropylamino)ethoxy-4-methoxyphenyl]-4-(3-cyanophenyl)piperazine-1-carboxamide: MS(ES) m/e 480.2 [M+H]⁺;

N-[3-(2-Diisopropylamino)ethoxy-4-methoxyphenyl]-4-[4-(ethoxycarbonyl)phenyl]piperazine-1-carboxamide: MS(ES) m/e 527.2 [M+H]⁺; and

N-[3-(2-Diisopropylamino)ethoxy-4-methoxyphenyl]-4-[2-(ethoxycarbonyl)phenyl]piperazine-1-carboxamide: MS(ES) m/e 527.2 [M+H]⁺.

### Example 23

### Preparation of 1′-(1-Methylethyl)spiro[benzofuran-3(2H),4′-piperidin]-5-amine

a) 5- and 7-nitrospiro[benzofuran-3(2H),4′-piperidine]

A solution of 1′-methyl-5- and 7-nitrospiro[benzofuran-3(2H),4′-piperidine] (WO 96/11934) (3 g, 12 mmol) and diisopropylethylamine (2.5 g, 19 mmol) in 1,2-dichloroethane (80 mL) was treated with 1-chloroethyl chloroformate (2.3 g, 16 mmol) at RT, stirred for 1 h, and heated to reflux for 20 min. The mixture was cooled, concentrated in vacuo, and the residue was dissolved in methanol and heated to reflux for 2 h, concentrated in vacuo, and the residue was partitioned between dichloromethane (250 mL) and 5% sodium bicarbonate (50 mL). The organic phase was washed with 5% sodium bicarbonate (50 mL) and the combined aqueous phase was extracted with dichloromethane (2×50 mL). The combined organic phase was dried Na₂SO₄) and concentrated to afford the title compound (2.65 g).

b) 1′-(tert-butoxycarbonyl)-5-nitrospiro[benzofuran-3(2H),4′-piperidine]

A solution of the compound of Preparation 2(a) (2.65 g, 1.13 mmol) in tetrahydrofuran (300 mL) was treated with di-tert-butyl dicarbonate (2.6 g, 12 mmol) and stirred at RT for 16 h. The mixture was concentrated in vacuo and the residue was crystallized from methanol to afford the title compound (2.1 g).

c) 5-nitrospiro[benzofuran-3(2H),4′-piperidine]

A solution of the compound of Preparation 2(b) (2.1 g, 6.3 mmol) in dichloromethane (50 mL) and trifluoroacetic acid (10 mL) was kept at RT for 5 h, concentrated in vacuo, and the residue was partitioned between dichloromethane (300 mL) and 5% sodium bicarbonate. The organic phase was washed with 5% sodium bicarbonate and the combined aqueous washes were extracted with dichloromethane. The combined organic phase was dried (Na₂SO₄) and concentrated in vacuo to give the title compound (1.45 g). MS(ES) m/e 235.1[+H]⁺.

d) 1′-(1-methylethyl)-5-nitrospiro[benzofuran-3(2H),4′piperidine]

A mixture of the compound of Preparation 2(c) (1.45 g, 6.2 mmol), powdered potassium carbonate (0.86 g, 6.2 mmol) and dimethylformamide (50 mL) containing 2-iodopropane (1.1 g, 6.4 mmol) was stirred and heated to 50° C. for 4 h, treated with 2-iodopropane (0.17 g, 1 mmol) at 50° C. for 90 min, and treated with 2-iodopropane (0.1 g, 1 mmol) at 50° C. for 2 h. The mixture was concentrated in vacuo and the residue was partitioned between ethyl acetate (20 mL) and water (20 mL). The organic phase was washed, dried (MgSO₄), concentrated in vacuo, and the residue was chromatographed (silica gel, 5% methanol:dichloromethane) to give the title compound (0.85 g).

e) 1′-(1-methylethyl)spiro[benzofuran-3(2H),4′-piperidin]-5-amine

A solution of the compound of Preparation 2(d) (0.78 g, 2.8 mmol) in methanol (250 mL) containing 10% palladium-on-carbon (0.375 g) was shaken in a hydrogen atmosphere (40 psi) for 40 min, filtered, and concentrated in vacuo to afford the title compound (0.6 g).

### Example 24

Following the procedure of Example 2, except substituting the compound of Example 23(e) for 3-(2-diisopropylamino)ethoxy-4-methoxyaniline, gave the following compound:

N-[2,3-dihydro-1′-isopropyl-spiro[benzofuran-5-yl-3,4′-piperidine]]-4-(2,3-dimethylphenyl)piperazine-1-carboxamide: MS(ES) m/e 463.1 [M+H]⁺.

### Examples 25-46

Following the procedure of Example 2, except substituting 1-(3-methylphenyl)piperazine, 1-(4-methylphenyl)piperazine, 1-(2,5-dimethylphenyl)piperazine, 1-(3,4-dimethylphenyl)piperazine, 1-(3,5-dichlorophenyl)piperazine, 1-(3-methoxyphenyl)piperazine, 1-(3,5-dimethoxyphenyl)piperazine, 1-[3-(ethoxycarbonyl)phenyl]piperazine, 1-(2-cyanophenyl)piperazine, 1-(4-cyanophenyl)piperazine, 1-(2-pyridinyl)piperazine, 1-(4-pyridinyl)piperazine, 1-[4-chloro-3-(trifluoromethyl)phenyl]piperazine, 1-[2-methyl-3-(trifluoromethyl)phenyl]piperazine, 1-(1-naphthalenyl)piperazine, 1-[1-(5,6,7,8-tetrahydronaphthalenyl]piperazine, 1-(1H-indol-4-yl)piperazine, 1-(4-methoxyphenyl)-3-methylpiperazine, 1-(5-chloro-2-methoxyphenyl)piperazine, 1-(3-hydroxyphenyl)piperazine, 1-(5-chloro-2-methylphenyl)piperazine, and 1-(3-chloro-2-methylphenyl)piperazine for 1-(2,3-dimethylphenyl)piperazine, gave the title compounds:

N-[3-(2-diisopropylamino)ethoxy-4-methoxyphenyl]-4-(3-methylphenyl)piperazine-1-carboxamide: MS(ES) m/e 469.4 [M+H]⁺;

N-[3-(2-diisopropylamino)ethoxy-4-methoxyphenyl]-4-(4-methylphenyl)piperazine-1-carboxamide: MS(ES) m/e 469.4 [M+H]⁺;

N-[3-(2-diisopropylamino)ethoxy-4-methoxyphenyl]-4-(2,5-dimethylphenyl)piperazine-1-carboxamide: MS(ES) m/e 483.4 [M+H]⁺;

N-[3-(2-diisopropylamino)ethoxy-4-methoxyphenyl]-4-(3,4-dimethylphenyl)piperazine-1-carboxamide: MS(ES) m/e 483.4 [M+H]⁺;

N-[3-(2-diisopropylamino)ethoxy-4-methoxyphenyl]-4-(3,5-dichlorophenyl)piperazine-1-carboxamide: MS(ES) m/e 523.4 [M+H]⁺;

N-[3-(2-diisopropylamino)ethoxy-4-methoxyphenyl]-4-(3-methoxyphenyl)piperazine-1-carboxamide: MS(ES) m/e 485.4 [M+H]⁺;

N-[3-(2-diisopropylamino)ethoxy-4-methoxyphenyl]-4-(3,5-dimethoxyphenyl)piperazine-1-carboxamide: MS(ES) m/e 515.4 [M+H]⁺;

N-[3-(2-diisopropylamino)ethoxy-4-methoxyphenyl]-4-[3-(ethoxycarbonyl)phenyl]piperazine-1-carboxamide: MS(ES) m/e 527.4 [M+H]⁺;

N-[3-(2-diisopropylamino)ethoxy-4-methoxyphenyl]-4-(2-cyanophenyl)piperazine-1-carboxamide: MS(ES) m/e 480.4 [M+H]⁺;

N-[3-(2-diisopropylamino)ethoxy-4-methoxyphenyl]-4-(4-cyanophenyl)piperazine-1-carboxamide: MS(ES) m/e 480.4 [M+H]⁺;

N-[3-(2-diisopropylamino)ethoxy-4-methoxyphenyl]-4-(2-pyridinyl)piperazine-1-carboxamide: MS(ES) m/e 456.4 [M+H]⁺;

N-[3-(2-diisopropylamino)ethoxy-4-methoxyphenyl]-4-(4-pyridinyl)piperazine-1-carboxamide: MS(ES) m/e 456.4 [M+H]⁺;

N-[3-(2-diisopropylamino)ethoxy-4-methoxyphenyl]-4-[4-chloro-3-(trifluoromethyl)phenyl]piperazine-1-carboxamide: MS(ES) m/e 557.2 [M+H]⁺;

N-[3-(2-diisopropylamino)ethoxy-4-methoxyphenyl]-4-[2-methyl-3-(trifluoromethyl)phenyl]piperazine-1-carboxamide: MS(ES) m/e 537.4 [M+H]⁺;

N-[3-(2-diisopropylamino)ethoxy-4-methoxyphenyl]-4-(1-naphthalenyl)piperazine-1-carboxamide: MS(ES) m/e 505.4 [M+H]⁺;

N-[3-(2-diisopropylamino)ethoxy-4-methoxyphenyl]-4-[1-(5,6,7,8-tetrahydronaphthalenyl]piperazine-1-carboxamide: MS(ES) m/e 509.6 [M+H]⁺;

N-[3-(2-diisopropylamino)ethoxy-4-methoxyphenyl]-4-(1H-indol-4-yl)piperazine-1-carboxamide: MS(ES) m/e 494.4 [M+H]⁺;

-[3-(2-diisopropylamino)ethoxy-4-methoxyphenyl]-4-(4-methoxyphenyl)-3-methylpiperazine-1-carboxamide: MS(ES) m/e 499.4 [M+H]⁺;

N-[3-(2-diisopropylamino)ethoxy-4-methoxyphenyl]-4-(5-chloro-2-methoxyphenyl)piperazine-1-carboxamide: MS(ES) m/e 519.4 [M+H]⁺;

N-[3-(2-diisopropylamino)ethoxy-4-methoxyphenyl]-4-(3-hydroxyphenyl)piperazine-1-carboxamide: MS(ES) m/e 471.4 [M+H]⁺;

N-[3-(2-diisopropylamino)ethoxy-4-methoxyphenyl]-4-(5-chloro-2-methylphenyl)piperazine-1-carboxamide: MS(ES) m/e 503.4 [M+H]⁺; and

N-[3-(2-diisopropylamino)ethoxy-4-methoxyphenyl]-4-(3-chloro-2-methylphenyl)piperazine-1-carboxamide: MS(ES) m/e 503.4 [M+H]³⁰ .

### Example 47

### Preparation of 4-(2,3-Dimethylphenyl)-N-[4-methoxy-3-[1-(1-methylethyl)-4-piperidinyl]phenyl]-1-piperazinecarboxamide

Triphosgene (12.2 mg, 0.041 mmol) was added to a solution of the compound of Preparation 1(e) (31 mg, 0.125 mmol) in dichloromethane (1 mL). The mixture was , stirred for 30 min and then triethylamine ( 0.07 mL, 0.5 mmol) was added. The mixture was stirred an additional 1 h, treated with 1-(2,3-dimethylphenyl)piperazine (31.0 mg, 0.125 mmol), and the mixture stirred at RT overnight. The resultant mixture was concentrated in vacuo and the residue was purified by preparative BPLC (YMC CombiPrep ODS-A, 50×20 mm, 20 mL/min, A:0.1% trifluoroacetic acid in acetonitrile B:0.1% aqueous trifluoroacetic acid, A:10 to 90% during 10 min, UV detection at 254 nm) to give 30 mg (52%) of the title compound as a yellow oil. MS(ES) m/e 465.4 [+H]⁺.

### Example 48

### Preparation of 4-(2,3-Dichlorophenyl)-N-[4-methoxy-3-1-(1-methylethyl)-4-piperidinyl]phenyl]-1-piperazinecarboxamide

Following the procedure of Example 47, except substituting 1-(2,3-dichlorophenyl)piperazine for 1-(2,3-dimethylphenyl)piperazine, gave the title compound. MS(ES) m/e 505.4 [M+H]⁺.

### Example 49

### Preparation of N-[3-(2-Diisopropylamino)ethoxy-4-methoxyphenyl]-4-(3-carboxyphenyl)piperazine-1-carboxamide

To a flask containing the compound of Example 32 (5.5 mg, 0.01 mmol) was added 0.5 ml ethanol and 0.3 N sodium hydroxide (0.1 ml, 0.03 mmol.). The mixture was stirred at RT overnight. The resultant mixture was concentrated in vacuo and the residue was purified by preparative BPLC (YMC CombiPrep ODS-A, 50×20 mm, 20 mL/min, A:0.1% trifluoroacetic acid in acetonitrile B:0.1% aqueous trifluoroacetic acid, A:10 to 90% during 10 min, UV detection at 254 nm) to give 1.0 mg (19%) of the title compound as a yellow oil. MS(ES) m/e 499.4 [M+H]⁺.

### Examples 50-51

Following the procedure of Example 49, except substituting the compounds of Examples 22 and 21 for the compound of Example 32 gave the title compounds:

N-[3-(2-diisopropylamino)ethoxy-4-methoxyphenyl]-4-(2-carboxyphenyl)piperazine-1-carboxamide: MS(ES) m/e 499.4 [M+H]⁺; and

N-[3-(2-diisopropylamino)ethoxy-4-methoxyphenyl]-4-(4-carboxyphenyl)piperazine-1-carboxamide: MS(ES) m/e 499.4 [M+H]⁺.

### Examples 52-61

Following the procedure of Example 2, except substituting 1-(3,4-dimethoxyphenyl)piperazine, 4-(2-benzothiazolyl)piperidine, 4(1H-indol-2-yl)-1-piperidine, 4-(4-chlorophenyl)-4-hydroxy-1-piperidine, 4-acetyl-4-(4-chlorophenyl)-1-piperidine, 4-(4-chlorophenyl)-4-cyano-1-piperidine, 4-(4-hydroxyphenyl)-1-piperidine, 1-(6-chloro-2-benzothiazolyl)piperazine, 1-(2-pyrazinyl)piperazine, and 1-[5-(trifluoromethyl)-2-pyridinyl]piperazine for 1-(2,3-dimethylphenyl)piperazine, gave the following compounds:

N-[3-(2-diisopropylamino)ethoxy-4-methoxyphenyl]-4-(3,4-dimethoxyphenyl)-1-piperazinecarboxamide: MS(ES) m/e 515.4 [M+H]⁺;

4-(2-benzothiazolyl)-N-[3-(2-diisopropylamino)ethoxy-4-methoxyphenyl]-1-piperidinecarboxamide: MS(ES) m/e 511.4 [M+H]⁺;

4-(1H-indol-2-yl)-N-[3-(2-diisopropylamino)ethoxy-4-methoxyphenyl]-1-piperidinecarboxamide: MS(ES) m/e 493.4 [M+H]⁺;

4-(4-chlorophenyl)-N-[3-(2-diisopropylamino)ethoxy-4-methoxyphenyl]-4-hydroxy-1-piperidinecarboxamide: MS(ES) m/e 504.4 [M+H]⁺;

4-acetyl-4-(4-chlorophenyl)-N-[3-(2-diisopropylamino)ethoxy-4-methoxyphenyl]-1-piperidinecarboxamide: MS(ES) m/e 496.4 [M+H]⁺;

4-(4-chlorophenyl)-4-cyano-N-[3-(2-diisopropylamino)ethoxy-4-methoxyphenyl]-1-piperidinecarboxamide: MS(ES) m/e 479.4 [M+H]⁺;

N-[3-(2-diisopropylamino)ethoxy-4-methoxyphenyl]-4-(4-hydroxyphenyl)-1-piperidinecarboxamide: MS(ES) m/e 470.4 [M+H]⁺;

4-(6-chloro-2-benzothiazolyl)-N-[3-(2-diisopropylamino)ethoxy-4-methoxyphenyl]-1-piperazinecarboxamide: MS(ES) m/e 546.4 [M+H]⁺;

N-[3-(2-diisopropylamino)ethoxy-4-methoxyphenyl]-4-(2-pyrazinyl)-1-piperazinecarboxamide: MS(ES) m/e 457.4 [M+H]⁺; and

N-[3-(2-diisopropylamino)ethoxy-4-methoxyphenyl]-4-[5-(trifluoromethyl)-2-pyridinyl]-1-piperazinecarboxamide: MS(ES) m/e 524.4 [M+H]⁺.

### Examples 62-96

Following the procedure of Example 47, except substituting 4-(2-benzothiazolyl)-1-piperidine, 4-(1H-indol-2-yl)-1-piperidine, 4-(4-chlorophenyl)-4-hydroxy-1-piperidine, 4-acetyl-4-(4-chlorophenyl)-1-piperidine, 4-(4-chlorophenyl)-4-cyano-1-piperidine, 4-(4-hydroxyphenyl)-1-piperidine, 1-(6-chloro-2-benzothiazolyl)piperazine, 1-(2-pyrazinyl)piperazine, 1-[5-(trifluoromethyl)-2-pyridinyl]piperazine, 1-(3,4-dimethoxyphenyl)piperazine, 1-(2-chlorophenyl)piperazine, 1-(3-chlorophenyl)-piperazine, 1-(4-chlorophenyl)piperazine, 1-(3,4-dichlorophenyl)piperazine, 1-(3,5-dichlorophenyl)piperazine, 1-(2,6-dimethylphenyl)piperazine, 1-(2,4-dimethylphenyl)-piperazine, 1-(3,5-dimethylphenyl)piperazine, 1-(3-methylphenyl)piperazine, 1-(2,5-dimethylphenyl)piperazine, 1-(3,4-dimethylphenyl)piperazine, 1-(5,6,7,8-tetrahydro-1-naphthalenyl)piperazine, 1-(2-methylphenyl)piperazine, 1-(5-chloro-2-methylphenyl-piperazine, 1-(3-chloro-2-methylphenyl)piperazine, 1-(3-chloro-2-methoxyphenyl)-piperazine, 1-[3-(trifluoromethyl)phenyl]piperazine, 1-[4-chloro-3-(trifluoromethyl)-phenyl]piperazine, 1-[2-methyl-3-(trifluoromethyl)phenyl]piperazine, 1-(3-methoxyphenyl)piperazine, 1-(3,5-dimethoxyphenyl)piperazine, 1-(2-cyanophenyl)piperazine 1-(4-cyanophenyl)piperazine, the compound of Preparation 3, and 1-(1H-indol-4-yl)piperazine for 1-(2,3-dimethylphenyl)piperazine, gave the following compounds:

4-(2-benzothiazolyl)-N-[4-methoxy-3-[1-(1-methylethyl)-4-piperidinyl]phenyl]-1-piperidinecarboxamide: MS(ES) m/e 493.4 [M+H]⁺;

4-(1H-indol-2-yl)-N-[4-methoxy-3-[1-(1-methylethyl)-4-piperidinyl]phenyl]-1-piperidinecarboxamide: MS(ES) m/e 475.4 [M+H]⁺;

4-(4-chlorophenyl)-N-[4-methoxy-3-[1-(1-methylethyl)-4-piperidinyl]phenyl]-4-hydroxy-1-piperidinecarboxamide: MS(ES) m/e 486.4 [M+H]⁺;

4-acetyl-4-(4-chlorophenyl)-N-[4-methoxy-3-[1-(1-methylethyl)-4-piperidinyl]phenyl]-1-piperidinecarboxamide: MS(ES) m/e 478.4 [M+H]⁺;

4-(4-chlorophenyl)-4-cyano-N-[4-methoxy-3-[1-(1-methylethyl)-4-piperidinyl]phenyl]-1-piperidinecarboxamide: MS(ES) m/e 461.4 [M+H]⁺;

4-(4-hydroxyphenyl)-N-[4methoxy-3-[1-(1-methylethyl)-4-piperidinyl]phenyl]-1-piperidinecarboxamide: MS(ES) m/e 452.2 [M+H]⁺;

4(6-chloro-2-benzothiazolyl)-N-[4-methoxy-3-[1-(1-methylethyl)-4-piperidinyl]phenyl]-1-piperazinecarboxamide: MS(ES) m/e 528.2 [M+H]⁺;

N-[4-methoxy-3-[1-(1-methylethyl)-4-piperidinyl]phenyl]-4-(2-pyrazinyl)-1-piperazinecarboxamide: MS(ES) m/e 439.2 [M+H]⁺;

N-[4-methoxy-3-[1-(1-methylethyl)-4-piperidinyl]phenyl]-4-[5-(trifluoromethyl)-2-pyridinyl]-1-piperazinecarboxamide: MS(ES) m/e 506.4 [M+H]⁺;

4-(3,4-dimethoxyphenyl)-N-[4-methoxy-3-[1-(1-methylethyl)-4-piperidinyl]phenyl]-1-piperazinecarboxamide: MS(ES) m/e 497.4 [M+H]⁺;

4-(2-chlorophenyl)-N-[4-methoxy-3-[1-(1-methylethyl)-4-piperidinyl]phenyl]-1-piperazinecarboxamide: MS(ES) m/e 471.4 [M+H]⁺;

4-(3-chlorophenyl)-N-[4-methoxy-3-[1-(1-methylethyl)-4-piperidinyl]phenyl]-1-piperazinecarboxamide: MS(ES) m/e 471.4 [M+H]⁺;

4-(4-chlorophenyl)-N-[4-methoxy-3-[1-(1-methylethyl)-4-piperidinyl]phenyl]-1-piperazinecarboxamide: MS(ES) m/e 471.4 [M+H]⁺;

4-(3,4-dichlorophenyl)-N-[4-methoxy-3-[1-(1-methylethyl)-4-piperidinyl]phenyl]-1-piperazinecarboxamide: MS(ES) m/e 505.4 [+H]⁺;

4-(3,5-dichlorophenyl)-N-[4-methoxy-3-[1-(1-methylethyl)-4-piperidinyl]phenyl]-1-piperazinecarboxamide: MS(ES) m/e 505.4 [M+H]⁺;

4-(2,6-dimethylphenyl)-N-[4-methoxy-3-[1-(1-methylethyl)-4-piperidinyl]phenyl]-1-piperazinecarboxamide: MS(ES) m/e 465.4 [M+H]⁺;

4-(2,4-dimethylphenyl)-N-[4-methoxy-3-[1-(1-methylethyl)-4-piperidinyl]phenyl]-1-piperazinecarboxamide: MS(ES) m/e 465.4 [M+H]⁺;

4-(3,5-dimethylphenyl)-N-[4-methoxy-3-[1-(1-methylethyl)-4-piperidinyl]phenyl]-1-piperazinecarboxamide: MS(ES) m/e 465.4 [M+H]⁺;

N-[4-methoxy-3-[1-(1-methylethyl)-4-piperidinyl]phenyl]-4-(3-methylphenyl)-1-piperazinecarboxamide: MS(ES) m/e 451.4 [M+H]⁺;

4-(2,5-dimethylphenyl)-N-[4-methoxy-3-[1-(1-methylethyl)-4-piperidinyl]phenyl]-1-piperazinecarboxamide: MS(ES) m/e 465.4 [M+H]⁺;

4-(3,4-dimethylphenyl)-N-[4-methoxy-3-[1-(1-methylethyl)-4-piperidinyl]phenyl]-1-piperazinecarboxamide: MS(ES) m/e 465.4 [+H]⁺;

N-[4-methoxy-3-[1-(1-methylethyl)-4-piperidinyl]phenyl]-4-(5,6,7,8-tetrahydro-1-naphthalenyl)-1-piperazinecarboxamide: MS(ES) m/e 491.4 [M+H]⁺;

N-[4-methoxy-3-[1-(1-methylethyl)-4-piperidinyl]phenyl]-4-(2-methylphenyl)-1-piperazinecarboxamide: MS(ES) m/e 451.4 [M+H]⁺;

4-(5-chloro-2-methylphenyl)-N-[4-methoxy-3-[1-(1-methylethyl)-4-piperidinyl]phenyl]-1-piperazinecarboxamide: MS(ES) m/e 485.4 [M+H]⁺;

4-(3-chloro-2-methylphenyl)-N-[4-methoxy-3-[1-(1-methylethyl)-4-piperidinyl]phenyl]-1-piperazinecarboxamide: MS(ES) m/e 485.4 [M+H]⁺;

4-(3-chloro-2-methoxyphenyl)-N-[4-methoxy-3-[1-(1-methylethyl)-4-piperidinyl]phenyl]-1-piperazinecarboxamide: MS(ES) m/e 501.4 [M+H]⁺;

N-[4-methoxy-3-[1-(1-methylethyl)-4-piperidinyl]phenyl]-4-[3-(trifluoromethyl)phenyl]1-piperazinecarboxamide: MS(ES) m/e 505.4 [M+H]⁺;

4-[4-chloro-3-(trifluoromethyl)phenyl]-N-[4-methoxy-3-[1-(1-methylethyl)-4-piperidinyl]phenyl]-1-piperazinecarboxamide: MS(ES) m/e 539.4 [M+H]⁺;

N-[4-methoxy-3-[1-(1-methylethyl)-4-piperidinyl]phenyl]-4-[2-methyl-3-(trifluoromethyl)phenyl]-1-piperazinecarboxamide: MS(ES) m/e 519.4 [M+H]⁺;

4-(3-methoxyphenyl)-N-[4-methoxy-3-[1-(1-methylethyl)-4-piperidinyl]phenyl]-1-piperazinecarboxamide: MS(ES) m/e 467.4 [M+H]⁺;

4-(3,5-dimethoxyphenyl)-N-[4-methoxy-3-[1-(1-methylethyl)-4-piperidinyl]phenyl]-1-piperazinecarboxamide: MS(ES) m/e 497.4 [M+H]⁺;

4-(2-cyanophenyl)-N-[4-methoxy-3-[1-(1-methylethyl)-4-piperidinyl]phenyl]-1-piperazinecarboxamide: MS(ES) m/e 462.4 [M+H]⁺;

4-(4-cyanophenyl)-N-[4-methoxy-3-[1-(1-methylethyl)-4-piperidinyl]phenyl]-1-piperazinecarboxamide: MS(ES) m/e 462.4 [M+H]⁺;

4-[3-(ethoxycarbonyl)phenyl]-N-[4-methoxy-3-[1-(1-methylethyl)-4-piperidinyl]phenyl]-1-piperazinecarboxamide: MS(ES) m/e 509.4 [M+H]⁺; and

4-(1H-indol-4-yl)-N-[4-methoxy-3-[1-(1-methylethyl)-4-piperidinyl]phenyl]-1-piperazinecarboxamide: MS(ES) m/e 476.4 [M+H]⁺.

### Example 97

### Preparation of 4-[3-(Ethoxycarbonyl)phenyl]-N-[3-[3-[bis(1-methylethyl)amino]propoxy]-4-methoxyphenyl]-1-piperazinecarboxamide

Following the procedure of Example 2, except substituting 3-(3-diisopropylamino)propoxy-4-methoxyaniline (WO 99101127) for 3-(2-diisopropylamino)ethoxy-4-methoxyaniline and substituting the compound of Preparation 3 for 1-(2,3-dimethylphenyl)piperazine, gave the title compound. MS(ES) m/e 541.4 [M+H]⁺.

### Example 98-99

### Preparation of 4-(2,3-Dimethylphenyl)-N-[4-methoxy-3-[1-cyclopentyl-4-piperidinyl]phenyl]-1-piperazinecarboxamide and 4-(2,3-Dimethylphenyl)-N-[1-(3-pentyl)-4-methoxy-3-[1-cyclopentyl-4-piperidinyl]phenyl]-1-piperazinecarboxamide

Following the general procedure of Example 47, except substituting the compounds of Preparation 4(b) and Preparation 5 for the compound of Preparation 1(e), gives the title compounds.

Biological Data:

CCR5 Receptor Binding Assay

CHO cell membranes (0.25×10⁶ cell equivalents) derived from CHO cells stably transfected with CCR5 were incubated with 0.3 ¹²⁵I-RANTES in a 96 well plate for 45 min. at room temperature (final reaction volume 200 uL). The reaction was terminated by filtration and the filters (GF/C) were washed twelve times with a solution of phosphate buffered saline containing 0.1% bovine serum albumin and 0.05% NaN₃. The radioactivity bound to filters was measured by liquid scintillation spectrometry. Non-specific binding was determined in the presence of unlabelled RANTES (10 or 30 nM) and averages 30-50% of total binding.

CCR5 Receptor Functional Assay

The cellular functional assay used to assess antagonist activity of compounds was RANTES-induced Ca²⁺ mobilization in RBL 2H3 cells stably expressing the hCCR5 receptor (RBL 2H3 hCCR5). Agonist activity is determined by Ca²⁺ mobilization in the same cells which is inhibitable by a selective CCR5 antagonist. Cells were grown to 80-100% confluency in T-150 flasks and washed with phosphate-buffered saline. Cells were lifted from the flasks by treating with 3 mL of 1 mM EDTA for 3 min. at room temperature and diluting to 2×10⁶ cells/mL with Krebs Ringer Henseleit buffer (KRH; 118 mM NaCl, 4.6 mM KCl, 25 mM NaHCO₃, 1 mM KH₂PO₄ and 11 mM glucose) containing 5 mM BEPES (pH 7.4), 1 mM CaCl₂, 1 mM MgCl₂ and 0.1% BSA and centrifuged at 200 g for 3 min. Cells were resuspended at 2×10⁶ cells/mL in the same buffer with 2 μM Fura-2AM, and incubated for 35 min. at 37° C. Cells were centrifuged at 200×g for 3 min. and resuspended in the same buffer without Fura-2AM, then incubated for 15 min. at 37° C. to complete the hydrolysis of intracellular Fura-2AM, and then centrifuged as before. Cells (10⁶ cells/mL) were resuspended in cold KRH with 5 mM HEPES (pH 7.4), 1 mM CaCl₂, 1 mM MgCl₂ and 0.1% gelatin and maintained on ice until assayed. For antagonist studies, aliquots (2 mL) of cells were pre-warmed at 37° C. for 5 min. in 3 mL plastic cuvettes and fluorescence measured in a fluorometer (Johnson Foundation Biomedical Group, Philadelphia, Pa., U.S.A.) with magnetic sting and temperature maintained at 37° C. Excitation was set at 340 nm and emission set at 510 nm. Various concentrations of antagonists or vehicle were added and fluorescence monitored for ˜15 sec to ensure that there was no change in baseline fluorescence, followed by the addition of 33 nM RANTES. Maximal Ca²⁺ attained after 33 nM RANTES stimulation was calculated as described by Grynliewicz et al., (1985). The percent of maximal RANTES-induced Ca²⁺ was determined for each concentration of antagonist and the IC₅₀, defined as the concentration of test compound that inhibits 50% of the maximal 33 nM RANTES response, obtained from the concentration-response curves (5-7 concentrations of antagonists).

The compounds of this invention show CCR5 receptor modulator activity having IC₅₀ values in the range of 0.0001 to 100 μM. The full structure/activity relationship has not yet been established for the compounds of this invention. However, given the disclosure herein, one of ordinary skill in the art can utilize the present assays in order to determine which compounds of formula (I) are modulators of the CCR5 receptor and which bind thereto with an IC₅₀ value in the range of 0.0001 to 100 μM.

All publications, including, but not limited to, patents and patent applications cited in this specification, are herein incorporated by reference as if each individual publication were specifically and individually indicated to be incorporated by reference herein as though fully set forth.

The above description fully discloses the invention including preferred embodiments thereof. Modifications and improvements of the embodiments specifically disclosed herein are within the scope of the following claims. Without further elaboration it is believed that one skilled in the art can, given the preceding description, utilize the present invention to its fullest extent. Therefore any examples are to be construed as merely illustrative and not a limitation on the scope of the present invention in any way. The embodiments of the invention in which an exclusive property or privilege is claimed are defined as follows.

What is claimed is:

1. A method of treating a CCR5-mediated disease state in mammals which comprises administering to a mammal in need of such treatment, an effective amount of a compound of formula (I) or a pharmaceutically acceptable salt or solvate thereof: 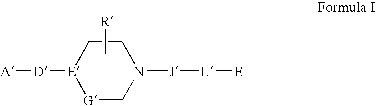
in which:
the basic nitrogen in moiety E may be optionally quaternized with C₁₋₆alkyl or is optionally present as the N-oxide;
A′ is aryl or heteroaryl, each of which is optionally substituted with one or more of R^{1′}; or A′ is aryl or heteroaryl fused to a saturated or partly unsaturated 5-7-membered ring to form a higher order ring moiety, which ring moiety optionally contains 1 or 2 heteroatoms selected from oxygen, nitrogen or sulfur, wherein nitrogen may be optionally substituted with hydrogen, C₁₋₆alkyl or C₃₋₇cycloalkyl; wherein the higher order ring moiety is optionally substituted with one or more of R^{1′};
R^{1′}is hydrogen, C₁₋₆allyl, C₂₋₆alkenyl, C₂₋₆alkynyl, C₃₋₇cycloalkyl, C₃₋₆cycloalkenyl, CH₂CF₃, aryl, aralkyl, (CH₂)_{a′}NR^{2′}R^{3′}, (CH₂)_{a′}NR^{2′}COR^{4′}, (CH₂)_{a′}NR^{2′}CO₂R^{5′}, (CH₂)_{a′} NR^{2′}SO₂R^{6′}, (CH₂)_{a′}CONR^{7′}R^{8′}, hydroxyC₁₋₆alkyl, C₁₋₄alkoxyalkyl (optionally substituted by a C₁₋₄alkoxy or hydroxy group), (CH₂)_{a′}CO₂C₁₋₆alkyl, (CH₂)_{b′}OC(O)R^{9′}, CR^{10′}═NOR^{11′}, CNR^{10′}═NOR^{11′}, COR^{12′}, CONR^{7′}R^{8′}, CONR^{7′}(CH₂)_{c′}OC₁₋₄alkyl, CONR^{7′}(CH₂)_{a′}CO₂R^{13′}, CONHNR^{14′}R^{15′}, CONR^{7′}SO₂R^{16′}, CO₂R^{17′}, cyano, trifluoromethyl, NR^{2′}R^{3′}, NR^{2′}COR^{4′}, NR^{18′}CO(CH₂)_{a′}NR^{18′}R^{19′}NR^{18′}CONR^{18′}R^{19′}, NR^{2′}CO₂R^{5′}, NR^{2′}SO₂R^{6′}, N═CNR^{18′}NR^{18′}R^{19′}, nitro, hydroxy, C₁₋₆alkoxy, OCF₃, hydroxyC₁₋₆alkoxy, C₁₋₆alkoxyC₁₋₆alkoxy, OC(O)NR^{20′}R^{21′}, SR^{22′}SOR^{23′}, SO₂R^{23′}SO₂NR^{20′}R^{21′} or halogen, or R^{1′} is a 5- to 7-membered ring containing 1 to 4 heteroatoms selected from nitrogen, oxygen, or sulfur, optionally substituted with hydrogen, C₁₋₆alkyl, C₃₋₇cycloalkyl, C₃₋₆cycloalkenyl, hydroxyC₁₋₆alkyl, (C₁₋₆alkyl)C₁₋₆alkyl, CONR^{7′}R^{8′}, CO₂R^{17′}, cyano, aryl, trifluoromethyl, nitro, hydroxy, C₁₋₆alkoxy, acyloxy, or halogen;
a′ is 1, 2, 3 or 4;
b′ is 0, 1, 2 or 3;
c′ is 1, 2 or 3;
R^{2′} and R^{3′} are independently hydrogen or C₁₋₆alkyl, or R^{2′} and R^{3′} together with the nitrogen to which they are attached, form a 5- to 6-membered heterocyclic ring which ring may be optionally substituted by an oxo group, or, when there are 6 ring members, the ring may optionally contain one oxygen or one sulfur atom;
R^{4′} is hydrogen, C₁₋₆alkyl or C₁₋₄alkoxyalkyl, or, when R^{1′} is NR^{2′}COR^{4′}, R^{4′} is (CH₂)₁₋₃ and forms a ring with A′;
R^{5′} is C₁₋₆alkyl;
R^{6′} is C₁₋₆alkyl or phenyl;
R^{7′} and R^{8′} are independently hydrogen or C₁₋₆alkyl, or R^{7′} and R^{8′} together with the nitrogen to which they are attached form a 5- to 6-membered saturated heterocyclic ring, wherein when there are 6 ring members, the ring may optionally contain one oxygen or one sulfur atom;
R^{9′} is C₁₋₄alkyl, optionally substituted by a C₁₋₆alkoxy;
R^{10′} and R^{11′} are independently hydrogen or C₁₋₆alkyl;
R^{12′} is hydrogen or C₁₋₆alkyl;
R^{13′} is hydrogen or C₁₋₆alkyl:
R^{14′} and R^{15′} are independently hydrogen or C₁₋₆alkyl;
R^{16′} is hydrogen or C₁₋₆alkyl;
R^{17′} is hydrogen or C₁₋₆alkyl optionally substituted with one or more substituents selected from C₁₋₆alkyl, C₁₋₆alkoxy, hydroxy, or NR^{2′}R^{3′};
R^{18′} and R^{19′} are independently hydrogen or C ₁₋₆alkyl;
R^{20′} and R^{21′} are independently hydrogen or C₁₋₆alkyl, or R^{20′} and R^{21′} together with the nitrogen to which they are attached form a 5- to 6-membered saturated heterocyclic ring which, when the ring is 6-membered, may optionally contain in the ring one oxygen or one sulfur atom.
R^{22′} is hydrogen or C₁₋₆alkyl;
R^{23′} is C₁₋₆allyl;
D′ is either a bond or represents [C(R^{24′})₂]_{a″}, [C(R^{24′})₂]_{a″}CO, CO, SO₂, CO[C(R^{24′})₂]_{a″}, O[C(R^{24′})₂]_{a″}, S[C(R^{24′})₂]_{a″}, O[C(R^{24′})₂]_{a″}CO, [C(R^{24′})₂]_{c″}OCO, NR^{25′}[C(R^{24′})₂]_{a″}, NR^{25′}[C(R^{24′})₂]_{a″}CO, [C(R^{24′})₂]_{c″}NR^{25′}CO, NR^{25″}CO[C(R^{24′})₂]_{a″}, NR^{25′}SO₂[C(R^{24′})₂]_{a″}, [C(R^{24′})₂]_{c″}NR^{25′}SO₂, CR^{24′}═CR^{24′}CO, C≡CCO, (C(R^{24′})₂)_{c″}SO₂, SO₂[C(R^{24′})₂]_{a″}, NR^{25′}[C(R^{24′})₂]_{a″}SO₂, NR^{25′}SO₂[C(R^{24′})₂]_{a″}SO₂, O[C(R^{24′})₂]_{a″}SO₂, SO₂NR^{25′}[C(R^{24′})₂]₁₋₂, [C(R^{24′})₂]_{b″}COO[C(R^{24′)}₂]₂, [C(R^{24′})₂]_{b″}CONR^{25′}[C(R^{24′})₂]₁₋₂; and when E′ and G′ together are CR^{27′}—C(R^{26′})₂, then D′ may further be O, NR^{25′}, CONR^{25′}, SO₂NR^{25′}, OCONR^{25′}, NR^{25′}COO, NR^{25′}CONR^{25′}, [CR^{24′})₂]_{a″}NR^{25′}[C(R^{24′})₂]_{b″}, [C(R^{24′})₂]_{a″}O[C(R^{24′})₂]_{b″}, CO[C(R^{24′})₂]_{a″}NR^{25′}, NR^{25′}[C(R^{24′})₂]_{a″}O, NR^{25′}[C(R^{24′})₂]_{a″}NR^{25′}, O[C(R^{24′})₂)]_{a″}NR^{25′}, O[C(R^{24′})₂]_{a″}O, CO[C(R^{24′})₂]_{a″}O, SO₂[C(R^{24′})₂]_{a″}NR^{25′}, SO₂[C(R^{24′})₂]_{a″}O, [C(R^{24′})₂]_{a″}SO₂NR^{25′}[C(R^{24′})₂]_{a″}CONR^{25′}, O[C(R^{24′})₂]_{a″}SO₂NR^{25′}, O[CR^{24′})₂]_{a″}CONR^{25′}, NR^{25′}[C(R^{24′})₂]_{a″}SO₂NR^{25′}, NR^{25′}[C(R^{24′})₂]_{a″}CONR^{25′}, NR^{25′}CO[C(R²⁴)₂]_{a″}NR^{25′}, NR^{25′}SO₂[C(R^{24′})₂]_{a″}NR^{25′}, (C(R^{24′})₂)_{a″}S(C(R^{24′})₂)_{b″}, COO, CR^{24′}OH, C(R^{24′})_{a″}CR^{24′}OH; and when E′ and G′ together are CR^{27′}—C(R^{26′})₂ or C═CR^{26′}, then D′ may further be CR^{24′}═CR^{24′} or C≡C; wherein a, is 1-6, b″ is 0-1, and c″ is 0-2;
R^{24′} is hydrogen or C₁₋₆alkyl;
R^{25′} is hydrogen or C₁₋₆alkyl;
B′ and G′ together are NC(R^{26′})₂, NC(R^{26′})₂C(R^{26′})₂, CR^{27′}C(R^{26′})₂ or C═CR^{26′};
R^{26′} is hydrogen or C₁₋₆alkyl;
R^{27′} is hydrogen, OR^{28′}, N^{28′}, CN, NO₂, R^{28′}, SR^{29′}, COR^{28′}, CHOHR^{28′}, CO₂R^{28′}, NHCOR^{28′}, NHCO₂R^{29′}, NHSO₂R^{29′}, or OCONHR^{28′};
R^{28′} is hydrogen, C₁₋₅alkyl, aryl or aralkyl;
R^{29′}is C₁₋₅alkyl, aryl or aralkyl;
R′ is one or more of hydrogen or C₁₋₆alkyl, or R′ is oxo;
J′ is CO or SO₂;
L′ is NR^{30′}, O or C(R^{30′})₂;
R^{30′} is hydrogen or C₁₋₆alkyl;
E represents a group (a): 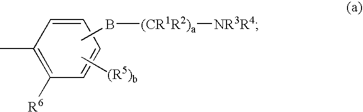
in which
B is oxygen, C≡C, S(O)_{c}, CR⁷═CR⁸, or CR⁷R⁸, or B is NR⁹;
R¹ and R² are independently hydrogen or C₁₋₆alkyl; alternatively B(CR¹R²)a is OCR¹R²CR¹(OH)CR¹R² or OCR¹R²CR¹(OCOCH₃)CR¹R²;
R³ and R⁴ are independently hydrogen, C₁₋₆alkyl, C₃₋₇cycloalkyl, aralkyl, or together with the nitrogen atom to which they are attached form an optionally substituted 5- to 7-membered heterocyclic ring which may contain an additional heteroatom selected from oxygen, nitrogen or sulfur, where optional substituents include C₁₋₆alkyl, aryl, CONR¹⁰R¹¹, NR¹⁰R¹¹, hydroxy, OCOR¹², NHCOCF₃, NHSO₂R¹³, NHCO₂R¹⁴, or NHCOC₀₋₆alkyl wherein the alkyl of NHCOC₀₋₆alkyl is optionally substituted by OH;
R⁵ is hydrogen, C₁₋₆alkyl, aryl, CN, CONR¹⁵R¹⁶, CO₂R¹⁷, trifluoromethyl, NHCO₂R¹⁸, hydroxy, C₁₋₆alkoxy, benzyloxy, OCH₂CO₂C₁₋₆alkyl, OCF₃, S(O)_{d}R¹⁹, SO₂NR²⁰R²¹ or halogen;
R⁶ is hydrogen, C₁₋₆alkyl, aryl, trifluoromethyl, hydroxy, C₁₋₆alkoxy or halogen, or R⁶ taken together with R^{30′} forms a group D where D is (CR²²R²³)ₑ or D is (CR²²R²³)_{f}—G where G is oxygen, sulfur or CR²²═CR²³, CR²²═N, ═CR²²O, ═CR²²S, or ═CR²²—NR²³;
R⁷, R⁸, R¹⁰, R¹¹, R¹², R¹⁵, R¹⁶, R¹⁷, R²⁰, R²¹, R²², and R²³ are independently hydrogen or C₁₋₆alkyl;
R⁹ is hydrogen, C₁₋₆alkyl, or phenylC₁₋₆alkyl;
R¹³, R¹⁴, R¹⁸, and R¹⁹ are independently C₁₋₆alkyl;
a is 1, 2, 3, or 4;
b is 1 or 2;
c and d are independently 0, 1 or 2;
e is 2, 3 or 4;
f is 0, 1, 2or 3;
alternatively, E represents a group (b): 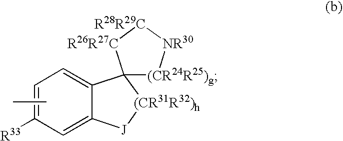
R²⁴, R²⁵, R²⁶, R²⁷, R²⁸, R²⁹, R³¹, and R³² are independently hydrogen or C₁₋₆alkyl;
R³⁰ is hydrogen, C₁₋₆alkyl, or C₃₋₇cycloalkyl;
R³³ is hydrogen, C₁₋₆alkyl, trifluoromethyl, hydroxy or halogen, or R³³ and R^{30′} together form a group —K— where K is (CR³⁴R³⁵)ᵢ or K is (CR³⁴R³⁵)ⱼ—M and M is oxygen, sulfur, CR³⁴═CR³⁵, CR³⁴═N, or N═N;
J is oxygen, CR³⁶R³⁷, or NR³⁸, or J is a group S(O)ₖ;
R³⁴, R³⁵, R³⁶, R³⁷, and R³⁸ are independently hydrogen or C₁₋₆alkyl;
g is 1, 2 or 3;
h is 1, 2 or 3;
i is 2, 3, or 4;
j is 0, 1, 2, or 3;
k is 0, 1 or 2;
alternatively, E represents a group (c): 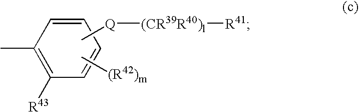
in which:
Q is oxygen, S(O)ₙ, CR⁴⁴═CR⁴⁵, CR⁴⁴R⁴⁵, or Q is NR⁴⁶;
R³⁹ and R⁴⁰ are independently hydrogen or C₁₋₆alkyl;
R⁴¹ is a group of formula (d): 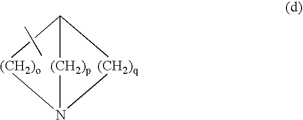
or R⁴¹ is a group of formula (e): 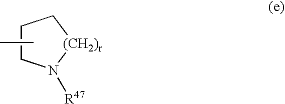
R⁴² is hydrogen, C₁₋₆alkyl, aryl, CN, CONR⁴⁸R⁴⁹, CO₂R⁵⁰, trifluoromethyl, NHCO₂R⁵¹, hydroxy, C₁₋₆alkoxy, benzyloxy, OCH₂CO₂C₁₋₆alkyl, OCF₃, S(O)ₛR⁵², SO₂NR⁵³R⁵⁴, or halogen;
R⁴³ is hydrogen or R⁴³ together with R^{30′} forms a group R where R is CR⁵⁵═CR⁵⁶, CR⁵⁵═CR⁵⁶CR⁵⁵R⁵⁶, or (CR⁵⁵R⁵⁶)ₜ;
R⁴⁴, R⁴⁵, R⁴⁶, R⁴⁸, R⁴⁹, R⁵⁰, R⁵³, R⁵⁴, R⁵⁵, and R⁵⁶ are independently hydrogen or C₁₋₆alkyl;
R⁴⁷ is hydrogen, C₁₋₆alkyl, or C₃₋₇ cycloalkyl;
R⁵¹ and R⁵² are independently C₁₋₆alkyl;
l is 0, 1, 2, or 3;
m is 1 or 2;
n is 0, 1, or 2
o, p, and q are independently integers having the value 1, 2, or 3;
r is 0,1, 2, or 3;
s is 0, 1, or 2;
t is 2 or 3;
alternatively, E represents a group (f): 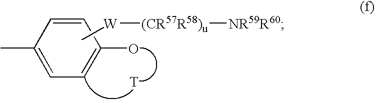
R⁵⁷ and R⁵⁸ are independently hydrogen or C₁₋₆alkyl;
R⁵⁹ and R⁶⁰ are independently hydrogen, C₁₋₆alkyl, C₃₋₇cycloalkyl, aralkyl, or together with the nitrogen atom to which they are attached form an optionally substituted 5- to 7-membered heterocyclic ring which may contain an additional heteroatom selected from oxygen, nitrogen or sulfur, where optional substituents include C₁₋₆alkyl, aryl, CONR⁶¹R⁶², NR⁶¹R⁶², hydroxy, OCOR⁶³, NHCOCF₃, NHSO₂R⁶⁴, NHCO₂R⁶⁵, or NHCOC₀₋₆alkyl wherein the alkyl of NHCOC₀₋₆alkyl is optionally substituted by OH;
T is —(CR⁶⁶R⁶⁷)ᵥ— or —O(CR⁶⁶R⁶⁷)_{w}—;
W is oxygen, S(O)ₓ, NR⁶⁸, or W is CR⁶⁹═CR⁷⁰ or CR⁶⁹R⁷⁰;
R⁶¹, R⁶², R⁶³, R⁶⁶, R⁶⁷ R⁶⁸, R⁶⁹, and R⁷⁰ are independently hydrogen or C₁₋₆alkyl;
R⁶⁴ and R⁶⁵ are independently C₁₋₆alkyl;
u is 1 to 4;
v is 2 or 3;
w is 1, 2, or 3;
x is 0, 1 or 2;
alternatively, E represents a group (g): 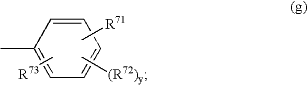
R⁷¹ is a 5- to 7-membered saturated or partially saturated heterocyclic ring containing a nitrogen atom and optionally a further 1 or 2 heteroatoms selected from nitrogen, oxygen or sulfur or R⁷¹ is an optionally substituted 6,6 or 6,5 bicyclic ring containing a nitrogen atom and optionally a further heteroatom selected from oxygen, nitrogen or sulfur, which ring systems may be optionally substituted with one or more of C₁₋₆alkyl and optionally substituted on nitrogen with hydrogen, C₁₋₆alkyl or C₃₋₇cycloalkyl;
R⁷² is hydrogen, C₁₋₆alkyl, aryl, CN, CONR⁷⁴R⁷⁵, CO₂R⁷⁶, trifluoromethyl, NHCO₂R⁷⁷, hydroxy, C₁₋₆alkoxy, benzyloxy, OCH₂CO₂C₁₋₆alkyl, OCF₃, S(O)_{z}R⁷⁸, SO₂NR⁷⁹R⁸⁰, or halogen;
R⁷³ is hydrogen, C₁₋₆alkyl, hydroxy, C₁₋₆alkoxy or halogen, or R⁷³ and R^{30′} taken together from a group —X— where X is (CR⁸¹R⁸²)ₐₐ or X is (CR⁸¹R⁸²)_{ab}—Y and Y is oxygen, sulfur or CR⁸¹═CR⁸²;
R⁷⁴, R⁷⁵, R⁷⁶, R⁷⁹, R⁸⁰, R⁸¹, and R⁸² are independently hydrogen or C₁₋₆alkyl;
R⁷⁷ and R⁷⁸ are independently C₁₋₆alkyl;
y is 1 or 2;
z is 0, 1, or 2;
aa is 2, 3 or 4;
ab is 0, 1, 2 or 3;
alternatively, E represents a group (h): 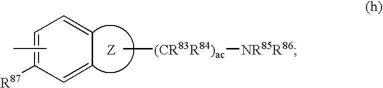
R⁸³ and R⁸⁴ are independently hydrogen or C₁₋₆alkyl;
R⁸⁵ and R⁸⁶ are independently hydrogen, C₁₋₆alkyl, C₃₋₇cycloalkyl, aralkyl, or together with the nitrogen atom to which they are attached form an optionally substituted 5- to 7-membered heterocyclic ring which may contain an additional heteroatom selected from oxygen, nitrogen or sulfur, where optional substituents include C₁₋₆alkyl, aryl, CONR⁸⁸R⁸⁹, NR⁹⁰R⁹¹, hydroxy, OCOR⁹², NHCOCF₃, NHSO₂R⁹³, NHCO₂R⁹⁴, or NHCOC₀₋₆alkyl wherein the alkyl of NHCOC₀₋₆alkyl is optionally substituted by OH;
R⁸⁷ is hydrogen or C₁₋₆alkyl, C₁₋₆alkoxy, or halogen, or R⁸⁷ together with R^{30′} forms a group —AA— where AA is (CR⁹⁵R⁹⁶)_{ad} or AA is (CR⁹⁵═CR⁹⁶)ₐₑ—AB and AB is oxygen, sulfur, CR⁹⁵═CR⁹⁶, CR⁹⁵═N, CR⁹⁵NR⁹⁶ or N═N;
Z is an optionally substituted 5 to 7-membered heterocyclic ring containing 1 to 3 heteroatoms selected from oxygen, nitrogen or sulfur;
R⁸⁸, R⁸⁹, R⁹⁰, R⁹¹, R⁹², R⁹⁵, and R⁹⁶ are independently hydrogen or C₁₋₆alkyl;
R⁹³ and R⁹⁴ are independently C₁₋₆alkyl;
ac is 0 to 4;
ad is 1, 2 or 3;
ae is 0, 1 or 2;
alternatively, E represents a group (i): 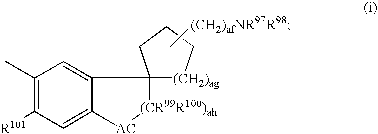
R⁹⁷ and R⁹⁸ are independently hydrogen, C₁₋₆alkyl, C₃₋₇cycloalkyl, aralkyl, or together with the nitrogen atom to which they are attached form an optionally substituted 5- to 7-membered heterocyclic ring which may contain an additional heteroatom selected from oxygen, nitrogen or sulfur, where optional substituents include C₁₋₆alkyl, aryl, CONR¹⁰²R¹⁰³, NR¹⁰⁴R¹⁰⁵, hydroxy, OCOR¹⁰⁶, NHCOCF₃, NHSO₂ R¹⁰⁷, NHCO₂R¹⁰⁸, or NHCOC₀₋₆alkyl wherein the alkyl of NHCOC₀₋₆alkyl is optionally substituted by OH;
R⁹⁹ and R¹⁰⁰ are independently hydrogen or C₁₋₆alkyl;
R¹⁰¹ is hydrogen or C₁₋₆alkyl or R¹⁰¹ and R^{30′} together form a group —AD— where AD is (CR¹⁰⁹R¹¹⁰)ₐᵢ or AD is (CR¹⁰⁹R¹¹⁰)ₐⱼ—AE and AE is oxygen, sulfur or CR¹⁰⁹═CR¹¹⁰;
AC is oxygen, CR¹¹¹R¹¹² or NR¹¹³ or AC is a group S(O)ₐₖ;
R¹⁰², R¹⁰³, R¹⁰⁴, R¹⁰⁵, R¹⁰⁶, R¹⁰⁹, R¹¹⁰, R¹¹¹, R¹¹², and R¹¹³ are independently hydrogen or C₁₋₆alkyl;
R¹⁰⁷ and R¹⁰⁸ are independently C₁₋₆alkyl;
af is 0, 1, 2, 3, or 4;
ag is 1, 2, or 3;
ah is 1, 2, 3 or 4;
ai is 2, 3 or 4;
aj is 0, 1, 2, or 3; and
ak is 0, 1 or 2.

2. The method as claimed in claim 1, wherein the compound of formula (I) is selected from:
N-[3-(2-Diisopropylamino)ethoxy-4-methoxyphenyl]-4-phenyl-1,2,3,6-tetrahydropyridine-1-carboxamide;
N-[3-(2-Diisopropylamino)ethoxy-4-methoxyphenyl]-4-phenylpiperazine-1-carboxamide;
N-[3-(2-Diisopropylamino)ethoxy-4-methoxyphenyl]-4-(2-methylphenyl)piperazine-1-carboxamide;
N-[3-(2-Diisopropylamino)ethoxy-4-methoxyphenyl]-4-(2,3-dimethylphenyl)piperazine-1-carboxamide;
N-[3-(2-Diisopropylamino)ethoxy-4-methoxyphenyl]-4-(2,3-dichlorophenyl)piperazine-1-carboxamide;
N-[3-(2-diisopropylamino)ethoxy-4-methoxyphenyl]-4-(2-acetamido-methylphenyl)piperazine-1-carboxamide;
N-[3-(2-Diisopropylamino)ethoxy-4-methoxyphenyl]-4-(3-trifluoromethylphenyl)piperazine-1-carboxamide;
N-[3-(2-diisopropylamino)ethoxy-4-methoxyphenyl]4-(2-methoxyphenyl)piperazine-1-carboxamide;
N-[3-(2-Diisopropylamino)ethoxy-4-methoxyphenyl]-4-(2-chlorophenyl)piperazine-1-carboxamide;
N-[3-(2-Diisopropylamino)ethoxy-4-methoxyphenyl]-4-(3-chlorophenyl)piperazine-1-carboxamide;
N-[3-(2-Diisopropylamino)ethoxy-4-methoxyphenyl]-4-(4-chlorophenyl)piperazine-1-carboxamide;
N-[3-(2-Diisopropylamino)ethoxy-4-methoxyphenyl]-4-(2,6-dimethylphenyl)piperazine-1-carboxamide;
N-[3-(2-Diisopropylamino)ethoxy-4-methoxyphenyl]-4-(3,4-dichlorophenyl)piperazine-1-carboxamide;
N-[3-(2-Diisopropylamino)ethoxy-4-methoxyphenyl]-3-methyl-4-(3-methylphenyl)piperazine-1-carboxamide;
N-[3-(2-Diisopropylamino)ethoxy-4-methoxyphenyl]-4-(4-methoxyphenyl)piperazine-1-carboxamide;
N-[3-(2-Diisopropylamino)ethoxy-4-methoxyphenyl]-4-(2,4-dimethylphenyl)piperazine-1-carboxamide;
N-[3-(2-Diisopropylamino)ethoxy-4-methoxyphenyl]-3-methyl-4-phenylpiperazine-1-carboxamide;
N-[3-(2-Diisopropylamino)ethoxy-4-methoxyphenyl]-4-(3,4dihydro-2(1H)-quinolinone-6-yl)piperazine-1-carboxamide;
N-[3-(2-Diisopropylamino)ethoxy-4-methoxyphenyl]-4-(3,5-dimethylphenyl)piperazine-1-carboxamide;
N-[3-(2-Diisopropylamino)ethoxy-4-methoxyphenyl]-4-(3-cyanophenyl)piperazine-1-carboxamide;
N-[3-(2-Diisopropylamino)ethoxy-4-methoxyphenyl]-4-[4-(ethoxycarbonyl)phenyl]piperazine-1-carboxamide;
N-[3-(2-Diisopropylamino)ethoxy-4-methoxyphenyl]-4-[2-(ethoxycarbonyl)phenyl]piperazine-1-carboxamide;
N-[2,3dihydro-1′-isopropyl-spiro[benzofuran-5-yl-3,4′-piperidine]]-4-(2,3-dimethylphenyl)piperazine-1-carboxamide;
N-[3-(2-Diisopropylamino)ethoxy-4-methoxyphenyl]-4-(3-methylphenyl)piperazine-1-carboxamide;
N-[3-(2-Diisopropylamino)ethoxy-4-methoxyphenyl]-4-(4-methylphenyl)piperazine-1-carboxamide;
N-[3-(2-Diisopropylamino)ethoxy-4-methoxyphenyl]-4-(2,5-dimethylphenyl)piperazine-1-carboxamide;
N-[3-(2-Diisopropylamino)ethoxy-4-methoxyphenyl]-4-(3,4-dimethylphenyl)piperazine-1-carboxamide;
N-[3-(2-Diisopropylamino)ethoxy-4-methoxyphenyl]-4-(3,5-dichlorophenyl)piperazine-1-carboxamide;
N-[3-(2-Diisopropylamino)ethoxy-4-methoxyphenyl]-4-(3-methoxyphenyl)piperazine-1-carboxamide;
N-[3-(2-Diisopropylamino)ethoxy-4-methoxyphenyl]-4-(3,5-dimethoxyphenyl)piperazine-1-carboxamide;
N-[3-(2-Diisopropylamino)ethoxy-4-methoxyphenyl]-4-[3-(ethoxycarbonyl)phenyl]piperazine-1-carboxamide;
N-[3-(2-Diisopropylamino)ethoxy-4-methoxyphenyl]-4-(2-cyanophenyl)piperazine-1-carboxamide;
N-[3-(2-Diisopropylamino)ethoxy-4-methoxyphenyl]-4-(4-cyanophenyl)piperazine-1-carboxamide;
N-[3-(2-Diisopropylamino)ethoxy-4-methoxyphenyl]-4-(2-pyridinyl)piperazine-1-carboxamide;
N-[3-(2-Diisopropylamino)ethoxy-4-methoxyphenyl]-4-(4-pyridinyl)piperazine-1-carboxamide;
N-[³-(2-Diisopropylamino)ethoxy-4-methoxyphenyl]-4-[4-chloro-3-(trifluoromethyl)phenyl]piperazine-1-carboxamide;
N-[3-(2-Diisopropylamino)ethoxy-4-methoxyphenyl]-4-[2-methyl-3-(trifluoromethyl)phenyl]piperazine-1-carboxamide;
N-[3-(2-Diisopropylamino)ethoxy-4-methoxyphenyl]-4-(1-naphthalenyl)piperazine-1-carboxamide;
N-[3-(2-Diisopropylamino)ethoxy-4-methoxyphenyl]-4-[1-(5,6,7,8-tetrahydronaphthalenyl]piperazine-1-carboxamide;
N-[3-(2-Diisopropylamino)ethoxy-4-methoxyphenyl]-4-(1H-indol-4-yl)piperazine-1-carboxamide;
N-[3-(2-Diisopropylamino)ethoxy-4-methoxyphenyl]-4-(4-methoxyphenyl)-3-methylpiperazine-1-carboxamide;
N-[3-(2-Diisopropylamino)ethoxy-4-methoxyphenyl]-4-(5-chloro-2-methoxyphenyl)piperazine-1-carboxamide;
N-[3-(2-Diisopropylamino)ethoxy-4-methoxyphenyl]-4-(3-hydroxyphenyl)piperazine-1-carboxamide;
N-[3-(2-Diisopropylamino)ethoxy-4-methoxyphenyl]-4-(5-chloro-2-methylphenyl)piperazine-1-carboxamide;
N-[3-(2-Diisopropylamino)ethoxy-4-methoxyphenyl]-4-(3-chloro-2-methylphenyl)piperazine-1-carboxamide;
4-(2,3-Dimethylphenyl)-N-[4-methoxy-3-[1-(1-methylethyl)-4-piperidinyl]phenyl]-1-piperazinecarboxamide;
4-(2,3-Dichlorophenyl)-N-[4-methoxy-3-[1-(1-methylethyl)-4-piperidinyl]phenyl]-1-piperazinecarboxamide;
N-[3-(2-Diisopropylamino)ethoxy-4-methoxyphenyl]-4-(3-carboxyphenyl)piperazine-1-carboxamide;
N-[3-(2-Diisopropylamino)ethoxy-4-methoxyphenyl]-4-(2-carboxyphenyl)piperazine-1-carboxamide;
N-[3-(2-Diisopropylamino)ethoxy-4-methoxyphenyl]-4-(3,4-dimethoxyphenyl)-1-piperazinecarboxamide;
4-(2-Benzothiazolyl)-N-[3-(2-diisopropylamino)ethoxy-4-methoxyphenyl]-1-piperidinecarboxamide;
4-(2-Benzothiazolyl)-N-[4-methoxy-3-[1-(1-methylethyl)-4-piperidinyl]phenyl]-1-piperidinecarboxamide;
4-(1H-Indol-2-yl)-N-[3-(2-diisopropylamino)ethoxy-4-methoxyphenyl]-1-piperidinecarboxamide;
4-(1H-Indol-2-yl)-N-[4-methoxy-3-[1-(1-methylethyl)-4-piperidinyl]phenyl]-1-piperidinecarboxamide;
4-(4-Chlorophenyl)-N-[3-(2-diisopropylamino)ethoxy-4-methoxyphenyl]-4-hydroxy-1-piperidinecarboxamide;
4-(4-Chlorophenyl)-N-[4-methoxy-3-[1-(1-methylethyl)-4-piperidinyl]phenyl]-4-hydroxy-1-piperidinecarboxamide;
4-Acetyl-4-(4-chlorophenyl)-N-[3-(2-diisopropylamino)ethoxy-4-methoxyphenyl]-1-piperidinecarboxamide;
4-Acetyl-4-(4-chlorophenyl)-N-[4-methoxy-3-[1-(1-methylethyl)-4-piperidinyl]phenyl]-1-piperidinecarboxamide;
4-(4-Chlorophenyl)-4-cyano-N-[3-(2-diisopropylamino)ethoxy-4-methoxyphenyl]-1-piperidinecarboxamide;
4-(4-Chlorophenyl)-4cyano-N-[4-methoxy-3-[1-(1-methylethyl)-4-piperidinyl]phenyl]-1-piperidinecarboxamide;
N-[3-(2-Diisopropylamino)ethoxy-4-methoxyphenyl]-4-(4-hydroxyphenyl)-1-piperidinecarboxamide;
4-(4-Hydroxyphenyl)-N-[4-methoxy-3-[1-(1-methylethyl)-4-piperidinyl]phenyl]-1-piperidinecarboxamide;
4-(6-Chloro-2-benzothiazolyl)-N-[3-(2-diisopropylamino)ethoxy-4-methoxyphenyl]-1-piperazinecarboxamide;
4-(6-Chloro-2-benzothiazolyl)-N-[4-methoxy-3-[1-(1-methylethyl)-4-piperidinyl]phenyl]-1-piperazinecarboxamide;
N-[3-(2-Diisopropylamino)ethoxy-4-methoxyphenyl]-4-(2-pyrazinyl)-1-piperazinecarboxamide;
N-[4-Methoxy-3-[1-(1-methylethyl)-4-piperidinyl]phenyl]-4-(2-pyrazinyl)-1-piperazinecarboxamide;
N-[3-(2-Diisopropylamino)ethoxy-4-methoxyphenyl]-4-[5-(trifluoromethyl)-2-pyridinyl]-1-piperazinecarboxamide;
N-[4-Methoxy-3-[1-(1-methylethyl)-4-piperidinyl]phenyl]-4-[5-(trifluoromethyl)-2-pyridinyl]-1-piperazinecarboxamide;
4-(3,4-Dimethoxyphenyl)-N-[4-methoxy-3-[1-(1-methylethyl)-4-piperidinyl]phenyl]-1-piperazinecarboxamide;
4-(2-Chlorophenyl)-N-[4-methoxy-3-[1-(1-methylethyl)-4-piperidinyl]phenyl]-1-piperazinecarboxamide;
4-(3-Chlorophenyl)-N-[4-methoxy-3-[1-(1-methylethyl)-4-piperidinyl]phenyl]-1-piperazinecarboxamide;
4-(4-Chlorophenyl)-N-[4-methoxy-3-[1-(1-methylethyl)-4-piperidinyl]phenyl]-1-piperazinecarboxamide;
4-(3,4-Dichlorophenyl)-N-[4-methoxy-3-[1-(1-methylethyl)-4-piperidinyl]phenyl]-1-piperazinecarboxamide;
4-(3,5-Dichlorophenyl)-N-[4-methoxy-3-[1-(1-methylethyl)-4-piperidinyl]phenyl]-1-piperazinecarboxamide;
4-(2,6-Dimethylphenyl)-N-[4-methoxy-3-[1-(1-methylethyl)-4-piperidinyl]phenyl]-1-piperazinecarboxamide;
4-(2,4-Dimethylphenyl)-N-[4-methoxy-3-[1-(1-methylethyl)-4-piperidinyl]phenyl]-1-piperazinecarboxamide;
4-(3,5-Dimethylphenyl)-N-[4-methoxy-3-[1-(1-methylethyl)-4-piperidinyl]phenyl]-1-piperazinecarboxamide;
N-[4-Methoxy-3-[1-(1-methylethyl)-4-piperidinyl]phenyl]-4-(3-methylphenyl)-1-piperazinecarboxamide;
4-(2,5-Dimethylphenyl)-N-[4-methoxy-3-[1-(1-methylethyl)-4-piperidinyl]phenyl]-1-piperazinecarboxamide;
4-(3,4-Dimethylphenyl)-N-[4-methoxy-3-[1-(1-methylethyl)-4-piperidinyl]phenyl]-1-piperazinecarboxamide;
N-[4-Methoxy-3-[1-(1-methylethyl)-4-piperidinyl]phenyl]-4-(5,6,7,8-tetrahydro-1-naphthalenyl)-1-piperazinecarboxamide;
N-[4-Methoxy-3-[1-(1-methylethyl)-4-piperidinyl]phenyl]-4-(2-methylphenyl)-1-piperazinecarboxamide;
4-(5-Chloro-2-methylphenyl)-N-[4-methoxy-3-[1-(1-methylethyl)-4-piperidinyl]phenyl]-1-piperazinecarboxamide;
4-(3-Chloro-2-methylphenyl)-N-[4-methoxy-3-[1-(1-methylethyl)-4-piperidinyl]phenyl]-1-piperazinecarboxamide;
4-(3-Chloro-2-methoxyphenyl)-N-[4-methoxy-3-[1-(1-methylethyl)-4-piperidinyl]phenyl]-1-piperazinecarboxamide;
N-[4-Methoxy-3-[1-(1-methylethyl)-4-piperidinyl]phenyl]-4-[3-(trifluoromethyl)phenyl]1-piperazinecarboxamide;
4-[4Chloro-3-(trifluoromethyl)phenyl]-N-[4-methoxy-3-[1-(1-methylethyl)-4-piperidinyl]phenyl]-1-piperazinecarboxamide;
N-[4-Methoxy-3-[1-(1-methylethyl)-4-piperidinyl]phenyl]-4-[2-methyl-3-(trifluoromethyl)phenyl]-1-piperazinecarboxamide;
4-(3-Methoxyphenyl)-N-[4-methoxy-3-[1-(1-methylethyl)-4-piperidinyl]phenyl]-1-piperazinecarboxamide;
4-(3,5-Dimethoxyphenyl)-N-[4-methoxy-3-[1-(1-methylethyl)-4-piperidinyl]phenyl]-1-piperazinecarboxamide;
4-(2-Cyanophenyl)-N-[4-methoxy-3-[1-(1-methylethyl)-4-piperidinyl]phenyl]-1-piperazinecarboxamide;
4-(4-Cyanophenyl)-N-[4-methoxy-3-[1-(1-methylethyl)-4-piperidinyl]phenyl]-1-piperazinecarboxamide;
4-[3-(Ethoxycarbonyl)phenyl]-N-[4-methoxy-3-[1-(1-methylethyl)-4-piperidinyl]phenyl]-1-piperazinecarboxamide;
4-(1H-Indol-4-yl)-N-[4-methoxy-3-[1-(1-methylethyl)-4-piperidinyl]phenyl]-1-piperazinecarboxamide;
4-[3-(Ethoxycarbonyl)phenyl]-N-[3-(3-diisopropylamino)propoxy-4-methoxyphenyl]-1-piperazinecarboxamide; and
N-[3-(2-Diisopropylamino)ethoxy-4-methoxyphenyl]-4-(4-carboxyphenyl)piperazine-1-carboxamide.

3. The method as claimed in claim 1, wherein the compound of formula (I) is selected from:
N-[3-(2-Diisopropylamino)ethoxy-4-methoxyphenyl]-4-(2,3-dimethylphenyl)piperazine-1-carboxamide;
N-[3-(2-Diisopropylamino)ethoxy-4-methoxyphenyl]-4-(2,3-dichlorophenyl)piperazine-1-carboxamide;
N-[3-(2-Diisopropylamino)ethoxy-4-methoxyphenyl]-4-(2,4-dimethylphenyl)piperazine-1-carboxamide;
N-[2,3-Dihydro-1′-isopropyl-spiro[benzofuran-5-yl-3,4′-piperidine]]-4-(2,3-dimethylphenyl)piperazine-1-carboxamide;
N-[3-(2-Diisopropylamino)ethoxy-4-methoxyphenyl]-4-[3-(ethoxycarbonyl)phenyl]piperazine-1-carboxamide;
N-[3-(2-Diisopropylamino)ethoxy-4-methoxyphenyl]-4-[2-methyl-3-(trifluoromethyl)phenyl]piperazine-1-carboxamide;
N-[3-(2-Diisopropylamino)ethoxy-4-methoxyphenyl]-4-[1-(5,6,7,8-tetrahydronaphthalenyl]piperazine-1-carboxamide;
N-[3-(2-Diisopropylamino)ethoxy-4-methoxyphenyl]-4-(5-chloro-2-methoxyphenyl)piperazine-1-carboxamide;
4-(6-Chloro-2-benzothiazolyl)-N-[4-methoxy-3-[1-(1-methylethyl)-4-piperidinyl]phenyl]-1-piperazinecarboxamide;
4-(2-Chlorophenyl)-N-[4-methoxy-3-[1-(1-methylethyl)-4-piperidinyl]phenyl]-1-piperazinecarboxamide;
4-(3-Chlorophenyl)-N-[4-methoxy-3-[1-(1-methylethyl)-4-piperidinyl]phenyl]-1-piperazinecarboxamide;
4-(4-Chlorophenyl)-N-[4-methoxy-3-[1-(1-methylethyl)-4-piperidinyl]phenyl]-1-piperazinecarboxamide;
4-(3,4-Dichlorophenyl)-N-[4-methoxy-3-[1-(1-methylethyl)-4-piperidinyl]phenyl]-1-piperazinecarboxamide;
4-(3,5-Dichlorophenyl)-N-[4-methoxy-3-[1-(1-methylethyl)-4-piperidinyl]phenyl]-1-piperazinecarboxamide;
4-(2,4-Dimethylphenyl)-N-[4-methoxy-3-[1-(1-methylethyl)-4-piperidinyl]phenyl]-1-piperazinecarboxamide;
4-(3,5-Dimethylphenyl)-N-[4-methoxy-3-[1-(1-methylethyl)-4-piperidinyl]phenyl]-1-piperazinecarboxamide;
N-[4-Methoxy-3-[1-(1-methylethyl)-4-piperidinyl]phenyl]-4-(3-methylphenyl)-1-piperazinecarboxamide;
4-(2,5-Dimethylphenyl)-N-[4-methoxy-3-[1-(1-methylethyl)-4-piperidinyl]phenyl]-1-piperazinecarboxamide;
4-(3,4-Dimethylphenyl)-N-[4-methoxy-3-[1-(1-methylethyl)-4-piperidinyl]phenyl)-1-piperazinecarboxamide;
N-[4-Methoxy-3-[1-(1-methylethyl)-4-piperidinyl]phenyl]-4-(5,6,7,8-tetrahydro-1-naphthalenyl)-1-piperazinecarboxamide;
N-[4-Methoxy-3-[1-(1-methylethyl)-4-piperidinyl]phenyl]-4-(2-methylphenyl)-1-piperazinecarboxamide;
4-(5-Chloro-2-methylphenyl)-N-[4-methoxy-3-[1-(1-methylethyl) piperidinyl]phenyl]-1-piperazinecarboxamide;
4-(3-Chloro-2-methylphenyl)-N-[4-methoxy-3-[1-(1-methylethyl)-4-piperidinyl]phenyl]-1-piperazinecarboxamide;
4-(3-Chloro-2-methoxyphenyl)-N-[4-methoxy-3-[1-(1-methylethyl)-4-piperidinyl]phenyl]-1-piperazinecarboxamide;
N-[4-Methoxy-3-[1-(1-methylethyl)-4-piperidinyl]phenyl]-4-[3-(trifluoromethyl)phenyl]1-piperazinecarboxamide;
4-[4-Chloro-3-(trifluoromethyl)phenyl]-N-[4-methoxy-3-[1-(1-methylethyl)-4-piperidinyl]phenyl]-1-piperazinecarboxamide;
N-[4-Methoxy-3-[1-(1-methylethyl)-4-piperidinyl]phenyl]-4-[2-methyl-3-(trifluoromethyl)phenyl]-1-piperazinecarboxamide;
4-(3-Methoxyphenyl)-N-[4-methoxy-3-[1-(1-methylethyl)-4-piperidinyl]phenyl]-1-piperazinecarboxamide;
4-(3,5-Dimethoxyphenyl)-N-[4-methoxy-3-[1-(1-methylethyl)-4-piperidinyl]phenyl]-1-piperazinecarboxamide;
4-(2-Cyanophenyl)-N-[4-methoxy-3-[1-(1-methylethyl)-4-piperidinyl]phenyl]-1-piperazinecarboxamide;
4-[3-(Ethoxycarbonyl)phenyl]-N-[4-methoxy-3-[1-(1-methylethyl)-4-piperidinyl]phenyl]-1-piperazinecarboxamide;
4-(1H-Indol-4-yl)-N-[4-methoxy-3-[1-(1-methylethyl)-4-piperidinyl]phenyl]-1-piperazinecarboxamide;
N-[4-Methoxy-3-[4-cyano-1-(1-methylethyl)-4-piperidinyl]phenyl]-4-(5,6,7,8-tetrahydro-1-naphthalenyl)-1-piperazinecarboxamide;
4-[3-(Ethoxycarbonyl)phenyl]-N-[3-(3-diisopropylamino)propoxy-4-methoxyphenyl]-1-piperazinecarboxamide; and
N-[3-(2-Diisopropylamino)ethoxy-4-methoxyphenyl]-4-(3chloro-2-methylphenyl)piperazine-1-carboxamide.

4. The method as claimed in claim 1, wherein the compound of formula (I) is selected from:
4-(3,5-Dichlorophenyl)-N-[4-methoxy-3-[1-(1-methylethyl)-4-piperidinyl]phenyl]-1-piperazinecarboxamide;
N-[4-Methoxy-3-[1-(1-methylethyl)-4-piperidinyl]phenyl]-4-(5,6,7,8-tetrahydro-1-naphthalenyl)-1-piperazinecarboxamide;
4-(3-Chloro-2-methylphenyl)-N-[4-methoxy-3-[1-(1-methylethyl)-4-piperidinyl]phenyl]-1-piperazinecarboxamide;
N-[4-Methoxy-3-[1-(1-methylethyl)-4-piperidinyl]phenyl]-4-[2-methyl-3-(trifluoromethyl)phenyl]-1-piperazinecarboxamide; and
4-(1H-Indol-4-yl)-N-[4-methoxy-3-[1-(1-methylethyl)4-piperidinyl]phenyl]-1-piperazinecarboxamide; and
N-[3-(2-Diisopropylamino)ethoxy-4-methoxyphenyl]-4-[1-(5,6,7,8-tetrahydronaphthalenyl]piperazine-1-carboxamide.

5. The method as claimed in claim 1, wherein the disease is selected from COPD, asthma and atopic disorders (for example, atopic dermatitis and allergies), rheumatoid arthritis, sarcoidosis, or idiopathic pulmonary fibrosis and other fibrotic diseases, atherosclerosis, psoriasis, autoimmune diseases such as multiple sclerosis, treating and/or preventing rejection of transplanted organs, inflammatory bowel disease, and HIV infection.

6. The method as claimed in claim 1, wherein A′ is phenyl, 5,6,7,8-tetrahydro-1-naphthalenyl, or 1H-indol-4-yl; D′ is a bond, E′ and G′ together are NCH₂, R′ is hydrogen, J′ is CO, L′ is NH, and E is group (g).

7. The method as claimed in claim 6, wherein A′ is phenyl, and R^{1′} is methyl, chloro or trifluoromethyl substituted at the 2 and/or 3-positions, or R^{1′} is 2,4-dimethyl, 2-methoxy-5-chloro, 2-methyl, 3-ethoxycarbonyl, or 3,5-dichloro.

8. A compound or a pharmaceutically active salt or solvate thereof, selected from:
N-[3-(2-Diisopropylamino)ethoxy-4-methoxyphenyl]-4-phenyl-1,2,3,6-tetrahydropyridine-1-carboxamide;
N-[3-(2-Diisopropylamino)ethoxy-4-methoxyphenyl]-4-phenylpiperazine-1-carboxamide;
N-[3-(2-Diisopropylamino)ethoxy-4-methoxyphenyl]-4-(2-methylphenyl)piperazine-1-carboxamide;
N-[3-(2-Diisopropylamino)ethoxy-4-methoxyphenyl]-4-(2,3-dimethylphenyl)piperazine-1-carboxamide;
N-[3-(2-Diisopropylamino)ethoxy-4-methoxyphenyl]-4-(2,3-dichlorophenyl)piperazine-1-carboxamide;
N-[3-(2-diisopropylamino)ethoxy-4-methoxyphenyl]-4-(2-acetamido-methylphenyl)piperazine-1-carboxamide;
N-[3-(2-Diisopropylamino)ethoxy-4-methoxyphenyl]-4-(3-trifluoromethylphenyl)piperazine-1-carboxamide;
N-[3-(2-diisopropylamino)ethoxy-4-methoxyphenyl]-4-(2-methoxyphenyl)piperazine-1-carboxamide;
N-[3-(2-Diisopropylamino)ethoxy-4-methoxyphenyl]-4-(2-chlorophenyl)piperazine-1-carboxamide;
N-[3-(2-Diisopropylamino)ethoxy-4methoxyphenyl]-4-(3-chlorophenyl)piperazine-1-carboxamide;
N-[3-(2-Diisopropylamino)ethoxy-4-methoxyphenyl]-4-(4-chlorophenyl)piperazine-1-carboxamide;
N-[3-(2-Diisopropylamino)ethoxy-4-methoxyphenyl]-4-(2,6-dimethylphenyl)piperazine-1-carboxamide;
N-[3-(2-Diisopropylamino)ethoxy-4-methoxyphenyl]-4-(3,4-dichlorophenyl)piperazine-1-carboxamide;
N-[3-(2-Diisopropylamino)ethoxy-4-methoxyphenyl]-3-methyl-4-(3-methylphenyl)piperazine-1-carboxamide;
N-[3-(2-Diisopropylamino)ethoxy-4-methoxyphenyl]-4-(4-methoxyphenyl)piperazine-1-carboxamide;
N-[3-(2-Diisopropylamino)ethoxy-4-methoxyphenyl]-4-(2,4-dimethylphenyl)piperazine-1-carboxamide;
N-[3-(2-Diisopropylamino)ethoxy-4-methoxyphenyl]-3-methyl-4-phenylpiperazine-1-carboxamide;
N-[3-(2-Diisopropylamino)ethoxy-4-methoxyphenyl]-4-(3,4-dihydro-2(1H)-quinolinone-6-yl)piperazine-1-carboxamide;
N-[3-(2-Diisopropylamino)ethoxy-4-methoxyphenyl]-4-(3,5-dimethylphenyl)piperazine-1-carboxamide;
N-[3-(2-Diisopropylamino)ethoxy-4-methoxyphenyl]-4-(3-cyanophenyl)piperazine-1-carboxamide;
N-[3-(2-Diisopropylamino)ethoxy-4-methoxyphenyl]-4-[4-(ethoxycarbonyl)phenyl]piperazine-1-carboxamide;
N-[3-(2-Diisopropylamino)ethoxy-4-methoxyphenyl]-4-[2-(ethoxycarbonyl)phenyl]piperazine-1-carboxamide;
N-[2,3-dihydro-1′-isopropyl-spiro[benzofuran-5-yl-3,4′-piperidine]]-4-(2,3-dimethylphenyl)piperazine-1-carboxamide;
N-[3-(2-Diisopropylamino)ethoxy-4-methoxyphenyl]-4-(3-methylphenyl)piperazine-1-carboxamide;
N-[3-(2-Diisopropylamino)ethoxy-4-methoxyphenyl]-4-(4-methylphenyl)piperazine-1-carboxamide;
N-[3-(2-Diisopropylamino)ethoxy-4-methoxyphenyl]-4-(2,5-dimethylphenyl)piperazine-1-carboxamide;
N-[3-(2-Diisopropylamino)ethoxy-4-methoxyphenyl]-4-(3,4-dimethylphenyl)piperazine-1-carboxamide;
N-[3-(2-Diisopropylamino)ethoxy-4-methoxyphenyl]-4-(3,5-dichlorophenyl)piperazine-1-carboxamide;
N-[3-(2-Diisopropylamino)ethoxy-4-methoxyphenyl]-4-(3-methoxyphenyl)piperazine-1-carboxamide;
N-[3-(2-Diisopropylamino)ethoxy-4-methoxyphenyl]-4-(3,5-dimethoxyphenyl)piperazine-1-carboxamide;
N-[3-(2-Diisopropylamino)ethoxy-4-methoxyphenyl]-4-[3-(ethoxycarbonyl)phenyl]piperazine-1-carboxamide;
N-[3-(2-Diisopropylamino)ethoxy-4-methoxyphenyl]-4-(2-cyanophenyl)piperazine-1-carboxamide;
N-[3-(2-Diisopropylamino)ethoxy-4-methoxyphenyl]-4-(4-cyanophenyl)piperazine-1-carboxamide;
N-[3-(2-Diisopropylamino)ethoxy-4-methoxyphenyl]-4-(2-pyridinyl)piperazine-1-carboxamide;
N-[3-(2-Diisopropylamino)ethoxy-4-methoxyphenyl]-4-(4-pyridinyl)piperazine-1-carboxamide;
N-[3-(2-Diisopropylamino)ethoxy-4-methoxyphenyl]-4-[4-chloro-3-(trifluoromethyl)phenyl]piperazine-1-carboxamide;
N-[3-(2-Diisopropylamino)ethoxy-4-methoxyphenyl]-4-[2-methyl-3-(trifluoromethyl)phenyl]piperazine-1-carboxamide;
N-[3-(2-Diisopropylamino)ethoxy-4-methoxyphenyl]-4-(1-naphthalenyl)piperazine-1-carboxamide;
N-[3-(2-Diisopropylamino)ethoxy-4-methoxyphenyl]-4-[1-(5,6,7,8-tetrahydronaphthalenyl]piperazine-1-carboxamide;
N-[3-(2-Diisopropylamino)ethoxy-4-methoxyphenyl]-4-(1H-indol-4-yl)piperazine-1-carboxamide;
N-[3-(2-Diisopropylamino)ethoxy-4-methoxyphenyl]-4-(4-methoxyphenyl)-3-methylpiperazine-1-carboxamide;
N-[3-(2-Diisopropylamino)ethoxy-4-methoxyphenyl]-4-(5-chloro-2-methoxyphenyl)piperazine-1-carboxamide;
N-[3-(2-Diisopropylamino)ethoxy-4-methoxyphenyl]-4-hydroxyphenyl)piperazine-1-carboxamide;
N-[3-(2-Diisopropylamino)ethoxy-4-methoxyphenyl-4(5-chloro-2-methylphenyl)piperazine-1-carboxamide;
N-[3-(2-Diisopropylamino)ethoxy-4-methoxyphenyl]-4-(3-chloro-2-methylphenyl)piperazine-1-carboxamide;
4-(2,3-Dimethylphenyl)-N-[4-methoxy-3-[1-(1-methylethyl)-4-piperidinyl]phenyl]-1-piperazinecarboxamide;
4-(2,3-Dichlorophenyl)-N-[4-methoxy-3-[1-(1-methylethyl)-4-piperidinyl]phenyl]-1-piperazinecarboxamide;
N-[3-(2-Diisopropylamino)ethoxy-4-methoxyphenyl]-4-(3-carboxyphenyl)piperazine-1-carboxamide;
N-[3-(2-Diisopropylamino)ethoxy-4-methoxyphenyl]-4-(2-carboxyphenyl)piperazine-1-carboxamide;
N-[3-(2-Diisopropylamino)ethoxy-4-methoxyphenyl]-4(3,4-dimethoxyphenyl)-1-piperazinecarboxamide;
4-(2-Benzothiazolyl)-N-[3-(2-diisopropylamino)ethoxy-4-methoxyphenyl-1-piperidinecarboxamide;
4-(2-Benzothiazolyl)-N-[4-methoxy-3-[1-(1-methylethyl)-4-piperidinyl]phenyl]-1-piperidinecarboxamide;
4-(1H-Indol-2-yl)-N-[3-(2-diisopropylamino)ethoxy-4-methoxyphenyl]-1-piperidinecarboxamide;
4-(1H-Indol-2-yl)-N-[4-methoxy-3-[1-(1-methylethyl)-4-piperidinyl]phenyl]-1-piperidinecarboxamide;
4-(4-Chlorophenyl)-N-[3-(2-diisopropylamino)ethoxy-4-methoxyphenyl]-4-hydroxy-1-piperidinecarboxamide;
4-(4-Chlorophenyl)-N-[4-methoxy-3-[1-(1-methylethyl)-4piperidinyl]phenyl]-4-hydroxy-1-piperidinecarboxamide;
4-Acetyl-4-(4-chlorophenyl)-N-[3-(2-diisopropylamino)ethoxy-4-methoxyphenyl]-1-piperidinecarboxamide;
4-Acetyl-4-(4-chlorophenyl)-N-[4-methoxy-3-[1-(1-methylethyl)-4-piperidinyl]phenyl]-1-piperidinecarboxamide;
4-(4-Chlorophenyl)-4-cyano-N-[3-(2-diisopropylamino)ethoxy-4-methoxyphenyl]-1-piperidinecarboxamide;
4-(4-Chlorophenyl)-4-cyano-N-[4-methoxy-3-[1-(1-methylethyl)-4-piperidinyl]phenyl]-1-piperidinecarboxamide;
N-[3-(2-Diisopropylamino)ethoxy-4-methoxyphenyl]-4-(4-hydroxyphenyl)-1-piperidinecarboxamide;
4-(4-Hydroxyphenyl)-N-[4-methoxy-3-[1-(1-methylethyl)-4-piperidinyl]phenyl]-1-piperidinecarboxamide;
4-(6-Chloro-2-benzothiazolyl)-N-[3-(2-diisopropylamino)ethoxy-4-methoxyphenyl]-1-piperazinecarboxamide;
4-(6-Chloro-2-benzothiazolyl)-N-[4-methoxy-3-[1-(1-methylethyl)-4-piperidinyl]phenyl]-1-piperazinecarboxamide;
N-[3-(2-Diisopropylamino)ethoxy-4-methoxyphenyl]-4-(2-pyrazinyl)-1-piperazinecarboxamide;
N-[4-Methoxy-3-[1-(1-methylethyl)-4-piperidinyl]phenyl]phenyl]-4-(2-pyrazinyl)-1-piperazinecarboxamide;
N-[3-(2-Diisopropylamino)ethoxy-4-methoxyphenyl]-4-[5-(trifluoromethyl)-2-pyridinyl]-1-piperazinecarboxamide;
N-[4-Methoxy-3-[1-(1-methylethyl)-4-piperidinyl]phenyl]-4-[5-(trifluoromethyl)-2-pyridinyl]-1-piperazinecarboxamide;
4-(3,4-Dimethoxyphenyl)-N-[4-methoxy-3-[1-(1-methylethyl)-4-piperidinyl]phenyl]-1-piperazinecarboxamide.
4-(2-Chlorophenyl)-N-[4-methoxy-3-[1-(1-methylethyl)-4-piperidinyl]phenyl]-1-piperazinecarboxamide;
4-(3-Chlorophenyl)-N-[4-methoxy-3-[1-(1-methylethyl)-4-piperidinyl]phenyl]-1-piperazinecarboxamide;
4-(4-Chlorophenyl)-N-[4-methoxy-3-[1-(1-methylethyl)-4-piperidinyl]phenyl]-1-piperazinecarboxamide;
4-(3,4-Dichlorophenyl)-N-[4-methoxy-3-[1-(1-methylethyl)-4-piperidinyl]phenyl]-1-piperazinecarboxamide;
4-(3,5-Dichlorophenyl)-N-[4-methoxy-3-[1-(1-methylethyl)-4-piperidinyl]phenyl]-1-piperazinecarboxamide;
4-(2,6-Dimethylphenyl)-N-[4-methoxy-3-[1-(1-methylethyl)-4-piperidinyl]phenyl]-1-piperazinecarboxamide;
4-(2,4-Dimethylphenyl)-N-[4-methoxy-3-[1-(1-methylethyl)-4-piperidinyl]phenyl]-1-piperazinecarboxamide;
4-(3,5-Dimethylphenyl)-N-[4-methoxy-3-[ 1-(1-methylethyl)-4-piperidinyl]phenyl]-1-piperazinecarboxamide;
N-[4-Methoxy-3-[1-(1-methylethyl)-4-piperidinyl]phenyl]-4-(3-methylphenyl)-1-piperazinecarboxamide;
4-(2,5-Dimethylphenyl)-N-[4-methoxy-3-[1-(1-methylethyl)-4-piperidinyl]phenyl]-1-piperazinecarboxamide;
4-(3,4-Dimethylphenyl)-N-[4-methoxy-3-[1-(1-methylethyl)-4-piperidinyl]phenyl]-1-piperazinecarboxamide;
N-[4-Methoxy-3-[1-(1-methylethyl)-4-piperidinyl]phenyl]-4-(5,6,7,8-tetrahydro-1-naphthalenyl)-1-piperazinecarboxamide;
N-[4-Methoxy-3-[1-(1-methylethyl)-4-piperidinyl]phenyl]-4-(2-methylphenyl)-1-piperazinecarboxamide;
4-(5-Chloro-2-methylphenyl)-N-[4-methoxy-3-[1-(1-methylethyl)-4-piperidinyl]phenyl]-1-piperazinecarboxamide;
4-(3-Chloro-2-methylphenyl)-N-[4-methoxy-3-[1-(1-methylethyl)-4-piperidinyl]phenyl]-1-piperazinecarboxamide;
4-(3-Chloro-2-methoxyphenyl)-N-[4-methoxy-3-[1-(1-methylethyl)-4-piperidinyl]phenyl]-1-piperazinecarboxamide;
N-[4-Methoxy-3-[1-(1-methylethyl)-4-piperidinyl]phenyl]-4-[3-(trifluoromethyl)phenyl]1-piperazinecarboxamide;
4-[4-Chloro-3-(trifluoromethyl)phenyl]-N-[4-methoxy-3-[1-(1-methylethyl)-4-piperidinyl]phenyl]-1-piperazinecarboxamide;
N-[4-Methoxy-3-[1-(1-methylethyl)-4-piperidinyl]phenyl]-4-[2-methyl-3-(trifluoromethyl)phenyl]-1-piperazinecarboxamide;
4-(3-Methoxyphenyl)-N-[4-methoxy-3-[1-(1-methylethyl)-4-piperidinyl]phenyl]-1-piperazinecarboxamide;
4-(3,5-Dimethoxyphenyl)-N-[4-methoxy-3-[1-(1-methylethyl)-4-piperidinyl]phenyl]-1-piperazinecarboxamide;
4-(2-Cyanophenyl)-N-[4-methoxy-3-[1-(1-methylethyl)-4-piperidinyl]phenyl]-1-piperazinecarboxamide;
4-(4-Cyanophenyl)-N-[4-methoxy-3-[1-(1-methylethyl)-4-piperidinyl]phenyl]-1-piperazinecarboxamide;
4-[3-(Ethoxycarbonyl)phenyl]-N-[4-methoxy-3-[1-(1-methylethyl)-4-piperidinyl]phenyl]-1-piperazinecarboxamide;
4-(1H-Indol-4-yl)-N-[4-methoxy-3-[1-(1-methylethyl)-4-piperidinyl]phenyl]-1-piperazinecarboxamide;
4-[3-(Ethoxycarbonyl)phenyl]-N-[3-(3-diisopropylamino)propoxy-4-methoxyphenyl]-1-piperazinecarboxamide; and
N-[3-(2-Diisopropylamino)ethoxy-4-methoxyphenyl]-4-(4-carboxyphenyl)piperazine-1-carboxamide.

9. A compound or a pharmaceutically active salt or solvate thereof, selected from:
N-[3-(2-Diisopropylamino)ethoxy-4-methoxyphenyl]-4-(2,3-dimethylphenyl)piperazine-1-carboxamide;
N-[3-(2-Diisopropylamino)ethoxy-4-methoxyphenyl]-4-(2,3-dichlorophenyl)piperazine-1-carboxamide;
N-[3-(2-Diisopropylamino)ethoxy-4-methoxyphenyl]-4-(2,4-dimethylphenyl)piperazine-1-carboxamide;
N-[2,3-Dihydro-1′-isopropyl-spiro[benzofuran-5-yl-3,4′-piperidine]]-4-(2,3-dimethylphenyl)piperazine-1-carboxamide;
N-[3-(2-Diisopropylamino)ethoxy-4-methoxyphenyl]-4-[3-(ethoxycarbonyl)phenyl]piperazine-1-carboxamide;
N-[3-(2-Diisopropylamino)ethoxy-4-methoxyphenyl]-4-[2-methyl-3-(trifluoromethyl)phenyl]piperazine-1-carboxamide;
N-[3-(2-Diisopropylamino)ethoxy-4-methoxyphenyl]-4-[1-(5,6,7,8-tetrahydronaphthalenyl]piperazine-1-carboxamide;
N-[3-(2-Diisopropylamino)ethoxy-4-methoxyphenyl]-4-(5-chloro-2-methoxyphenyl)piperazine-1-carboxamide;
4-(6-Chloro-2-benzothiazolyl)-N-]-4-methoxy-3-[1-(1-methylethyl)-4-piperidinyl]phenyl]-1-piperazinecarboxamide;
4-(2-Chlorophenyl)-N-[4-methoxy-3-[1-(1-methylethyl)4-piperidinyl]phenyl]-1-piperazinecarboxamide;
4-(3-Chlorophenyl)-N-[4-methoxy-3-[1-(1-methylethyl)-4-piperidinyl]phenyl]-1-piperazinecarboxamide;
4-(4-Chlorophenyl)-N-[4-methoxy-3-[1-(1-methylethyl)-4-piperidinyl]phenyl]-1-piperazinecarboxamide;
4-(3,4-Dichlorophenyl)-N-[4-methoxy-3-[1-(1-methylethyl)-4-piperidinyl]phenyl]-1-piperazinecarboxamide;
4-(3,5-Dichlorophenyl)-N-[4-methoxy-3-[1-(1-methylethyl)4-piperidinyl]phenyl]-1-piperazinecarboxamide;
4-(2,4-Dimethylphenyl)-N-[4-methoxy-3-[1-(1-methylethyl)-4-piperidinyl]phenyl]-1-piperazinecarboxamide;
4-(3,5-Dimethylphenyl)-N-[4-methoxy-3-[1-(1-methylethyl)-4-piperidinyl]phenyl]-1-piperazinecarboxamide;
N-[4-Methoxy-3-[1-(1-methylethyl)-4-piperidinyl]phenyl]-4-(3-methylphenyl)-1-piperazinecarboxamide;
4-(2,5-Dimethylphenyl)-N-[4-methoxy-3-[1-(1-methylethyl)-4-piperidinyl]phenyl]-1-piperazinecarboxamide;
4-(3,4-Dimethylphenyl)-N-[4-methoxy-3-[1-(1-methylethyl)-4-piperidinyl]phenyl]-1-piperazinecarboxamide;
N-[4-Methoxy-3-[1-(1-methylethyl)4-piperidinyl]phenyl]-4-(5,6,7,8-tetrahydro-1-naphthalenyl)-1-piperazinecarboxamide;
N-[4-Methoxy-3-[1-(1-methylethyl)-4-piperidinyl]phenyl]-4-(2-methylphenyl)-1-piperazinecarboxamide;
4-(5-Chloro-2-methylphenyl)-N-[4-methoxy-3-[1-(1-methylethyl)-4-piperidinyl]phenyl]-1-piperazinecarboxamide;
4-(3-Chloro-2-methylphenyl)-N-[4-methoxy-3-[1-(1-methylethyl)-4-piperidinyl]phenyl]-1-piperazinecarboxamide;
4-(3-Chloro-2-methoxyphenyl)-N-[4-methoxy-3-[1-(1-methylethyl)-4-piperidinyl]phenyl]-1-piperazinecarboxamide;
N-[4-Methoxy-3-[1-(1-methylethyl)-4-piperidinyl]phenyl]-4-[3-(trifluoromethyl)phenyl]1-piperazinecarboxamide;
4-[4-Chloro-3-(trifluoromethyl)phenyl]-N-[4-methoxy-3-[1-(1-methylethyl)-4-piperidinyl]phenyl]-1-piperazinecarboxamide;
N-[4-Methoxy-3-[1-(1-methylethyl)-4-piperidinyl]phenyl]-4-[2-methyl-3-(trifluoromethyl)phenyl]-1-piperazinecarboxamide;
4-(3-Methoxyphenyl)-N-[4-methoxy-3-[1-(1-methylethyl)-4-piperidinyl]phenyl]-1-piperazinecarboxamide;
4-(3,5-Dimethoxyphenyl)-N-[4-methoxy-3-[1-(1-methylethyl)-4-piperidinyl]phenyl]-1-piperazinecarboxamide;
4-(2-Cyanophenyl)-N-[4-methoxy-3-[1-(1-methylethyl)-4-piperidinyl]phenyl]-1-piperazinecarboxamide;
4-[3-(Ethoxycarbonyl)phenyl]-N-[4-methoxy-3-[1-(1-methylethyl)-4-piperidinyl]phenyl]-1-piperazinecarboxamide;
4-(1H-Indol-4-yl)-N-[4-methoxy-3-[1-(1-methylethyl)-4-piperidinyl]phenyl]-1-piperazinecarboxamide;
N-[4-Methoxy-3-[4-cyano-1-(1-methylethyl)-4-piperidinyl]phenyl]-4-(5,6,7,8-tetrahydro-1-naphthalenyl)-1-piperazinecarboxamide;
4-[3-Ethoxycarbonyl)phenyl]-N-[3-(3-diisopropylamino)propoxy-4-methoxyphenyl]-1-piperazinecarboxamide; and
N-[3-(2-Diisopropylamino)ethoxy-4-methoxyphenyl]-4-(3-chloro-2-methylphenyl)piperazine-1-carboxamide.

10. A compound or a pharmaceutically active salt or solvate thereof, selected from:
4-(3,5-Dichlorophenyl)-N-[4-methoxy-3-[1-(1-methylethyl)-4-piperidinyl]phenyl]-1-piperazinecarboxamide;
N-[4-Methoxy-3-[1-(1-methylethyl)-4piperidinyl]phenyl]-4-(5,6,7,8-tetrahydro-1-naphthalenyl)-1-piperazinecarboxamide;
4-(3-Chloro-2-methylphenyl)-N-[4-methoxy-3-[1-(1-methylethyl)-4-piperidinyl]phenyl]-1-piperazinecarboxamide;
N-[4-Methoxy-3-[1-(1-methylethyl)-4-piperidinyl]phenyl]-4-[2-methyl-3-(trifluoromethyl)phenyl]-1-piperazinecarboxamide; and
4-(1H-Indol-4-yl)-N-[4-methoxy-3-[1-(1-methylethyl)-4-piperidinyl]phenyl]-1-piperazinecarboxamide; and
N-[3-(2-Diisopropylamino)ethoxy-4-methoxyphenyl]-4-[1-(5,6,7,8-tetrahydronaphthalenyl]piperazine-1-carboxamide.

11. A pharmaceutical composition comprising a compound as claimed in claim 8, 9 or 10, and a pharmaceutically acceptable carrier.

12. A process for making a compound as claimed in claims 8, 9, or 10, comprising for compounds wherein L′ is NR^{30′},
a) treating a compound of formula (II): 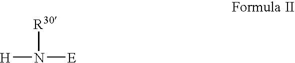
wherein R^{30′} is hydrogen or C₁₋₆alkyl, with triphosgene under basic conditions to form a mixture; and
b) adding to the mixture a compound of formula (III): 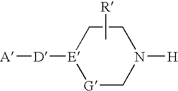
wherein A′, D′, E′, G′ and R′ are as defined in claim 1.

13. A compound selected from:
4-methoxy-3-[1-(1-methylethyl)-4-piperidinyl]benzenamine;
4-methoxy-3-[1-cyclopentyl-4-piperidinyl]benzenamine; and
4-methoxy-3-[1-(3-pentyl)-4-piperidinyl]benzenamine.
